(19) United States
(12) Patent Application Publication
(10) Pub. No.: US 2023/0399661 A1
(43) Pub. Date: Dec. 14, 2023

(54) SHUTTLE AGENT PEPTIDES OF MINIMAL LENGTH AND VARIANTS THEREOF ADAPTED FOR TRANSDUCTION OF CAS9-RNP AND OTHER NUCLEOPROTEIN CARGOS

(71) Applicant: FELDAN BIO INC., Québec (CA)

(72) Inventors: David GUAY, Québec (CA); Stéphanie HALLÉE, Ste-Catherine-de-la-Jacques-Cartier (CA); Xavier BARBEAU, Lévis (CA); Thomas DEL'GUIDICE, Québec (CA); Jean-Pascal LEPETIT-STOFFAES, Québec (CA)

(21) Appl. No.: 18/033,280

(22) PCT Filed: Oct. 22, 2021
(86) PCT No.: PCT/CA2021/051490

§ 371 Date: Apr. 21, 2023

Related U.S. Application Data
(60) Provisional application No. 63/104,340, filed on Oct. 22, 2020.

(51) Int. Cl. C12N 15/90; C12N 9/22; C12N 15/11; C07K 19/00

(52) U.S. Cl. CPC C12N 15/907; C12N 9/22; C12N 15/11; C07K 19/00; C12N 2310/20; C12N 2320/32; C07K 2319/10; C07K 2319/09

(57) ABSTRACT

Compositions and methods for delivering nucleoprotein cargos such as Cas9-RNP genome editing and ABE-Cas9-RNP base editing complexes to the cytosolic/nuclear compartment of eukaryotic cells via synthetic peptide shuttle agents are described herein. Also described herein are shortened synthetic peptide shuttle agents having a length of less than 20 amino acids having defined geometries associated with cargo transduction activity. The synthetic peptide shuttle agents are peptides comprising an amphipathic alpha-helical motif having both a positively-charged hydrophilic outer face and a hydrophobic outer face, wherein the synthetic peptide shuttle agent is independent from or is not covalently linked to the cargoes. Shuttle agents engineered for increased resistance to inhibition by nucleoproteins and/or extracellular DNA/RNA are also described herein.

### CROSS-REFERENCE TO RELATED APPLICATIONS

This application is a U.S. National Stage Application of PCT Application No. PCT/CA2021/051490, filed Oct. 22, 2021, which claims the benefit of U.S. Application No. 63/104,340, filed Oct. 22, 2020, each of which are incorporated herein by reference in their entirety.

The present description relates to the intracellular delivery of nucleoprotein cargoes via peptide-based delivery systems. More specifically, the present description relates to the use of synthetic peptide shuttle agents for the intracellular delivery of nucleoprotein cargoes such as Cas9-RNPs, as well as synthetic peptide shuttle agents engineered for increased resistance to inhibition by nucleoproteins and/or extracellular DNA/RNA.

The present description refers to a number of documents, the contents of which are herein incorporated by reference in their entirety.

### SEQUENCE LISTING

The instant application contains a Sequence Listing which has been submitted electronically in .txt format and is hereby incorporated by reference in its entirety. Said .txt copy, created on Apr. 17, 2023, is named 49446_706_831_SL txt and is 135,663 bytes in size.

### BACKGROUND

Genome editing using CRISPR-Cas enzymes offer great therapeutic potential but off-target genome edits represent a safety concern. Direct intracellular delivery of ribonucleoprotein (RNP) genome editing complexes are preferable over the use of DNA delivery because of the speed of genome editing and rapid clearance of the RNP afterwards. Conventional methods rely on lipofection or electroporation for RNP delivery, which have their limitations for therapeutic uses. RNP conjugation to cell-penetrating peptides have also been explored with limited success. Improved technologies for intracellular delivery of RNPs are thus highly desirable.

### SUMMARY

Synthetic peptide shuttle agents represent a recently defined family of peptides previously reported to transduce proteinaceous cargoes quickly and efficiently to the cytosol and/or nucleus of a wide variety of target eukaryotic cells. In contrast to traditional cell penetrating peptide-based intracellular delivery strategies, synthetic peptide shuttle agents are preferably not covalently linked to their polypeptide cargoes at the moment of delivery across the plasma membrane. In fact, covalently linking shuttle agents to their cargoes in a non-cleavable manner generally has a negative effect on their transduction activity. The first generation of such peptide shuttle agents was described in WO/2016/161516, wherein the peptide shuttle agents comprise an endosome leakage domain (ELD) operably linked to a cell penetrating domain (CPD). WO/2018/068135 subsequently described further synthetic peptide shuttle agents rationally-designed based on a set of fifteen design parameters for the sole purpose of improving the rapid transduction of proteinaceous cargoes, while reducing toxicity of the first generation peptide shuttle agents.

The majority of first and second generation shuttle agents are peptides at least twenty amino acids in length. Shuttle agent truncation experiments were undertaken herein to identify minimal fragments of first- and second-generation synthetic peptide shuttle agents sufficient for cargo transduction activity. These experiments revealed that C-terminal truncations were generally more tolerated than N-terminal truncations, with C-terminal truncations often retaining substantial cargo transduction activity when the N-terminal fragment is predicted to adopt a “core” region corresponding to an amphipathic cationic alpha helical structure when in solution at physiological conditions (e.g., at neutral pH). Common physiochemical properties of this core region and/or sub-20 amino acid shuttle agents are described herein.

In one aspect, described herein are synthetic peptide shuttle agents less than 20 amino acids in length having cargo transduction activity and their use for delivering a variety of cargoes in eukaryotic cells. The shuttle agents generally comprise a helical region comprising an amphipathic helix harboring: a cluster of hydrophobic amino acid residues on one side of the helix defining a hydrophobic angle of 140° to 280° in Schiffer-Edmundson's wheel representation, and a cluster of positively charged residues on the other side of the helix defining a positively charged angle of 40° to 160° in Schiffer-Edmundson's wheel representation.

While first- and second-generation shuttle agents efficiently deliver Cpf1-RNP (Cas12a-RNP) genome editing complexes to the nucleus of eukaryotic cells, they are shown herein to be less efficient at delivering Cas9-RNPs. While sharing similar sizes (SpCas9, 170 kDa and AsCpfl, 156 kDa), a major difference between the two enzymes likely influencing delivery is the net negative charge density contributed by their respective guide RNAs. AsCpfl uses a simple crRNA (CRISPR RNA) (˜42 nucleotides), and SpCas9 requires a crRNA and a tracrRNA (trans-activating crRNA) (˜100 nucleotides). Described herein are synthetic peptide shuttle agents suitable for improved delivery of Cas-RNPs, which include shorter peptides, as well as peptides having reduced cationic charge density in one or both flanking segments.

In further aspects, described herein is a composition comprising a nucleoprotein cargo for intracellular delivery and a synthetic peptide shuttle agent independent from or not covalently linked to said nucleoprotein cargo, the synthetic peptide shuttle agent being a peptide comprising an amphipathic alpha-helical motif having both a positively-charged hydrophilic outer face and a hydrophobic outer face, wherein synthetic peptide shuttle agent increases cytosolic/nuclear delivery of said nucleoprotein cargo in eukaryotic cells as compared to in the absence of the synthetic peptide shuttle agent. In embodiments, the nucleoprotein cargo is a deoxyribonucleoprotein (DNP) or ribonucleoprotein (RNP) complex such as Cas9-RNP.

In some embodiments, the shuttle agents described herein may comprise a fragment of a parent shuttle agent as defined herein, wherein the fragment retains cargo transduction activity and comprises an amphipathic alpha-helical motif having both a positively-charged hydrophilic outer face and a hydrophobic outer face. In some embodiments, the shuttle agents described herein may comprise a variant of a parent shuttle agent as defined herein, wherein the variant retains cargo transduction activity and differs (or differs only) from the shuttle agent by having a reduced C-terminal positive charge density relative to the parent shuttle agent (e.g., by substituting one or more cationic residues, such as K/R, with non-cationic residues, preferably non-cationic hydrophilic residues). In some embodiments, the shuttle agent fragments and/or variants described herein have increased resistance to inhibition by nucleoproteins and/or extracellular DNA/RNA, and/or have increased transduction activity for nucleoprotein cargoes.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “protein” or “polypeptide” or “peptide” means any peptide-linked chain of amino acids, which may or may not comprise any type of modification (e.g., chemical or post-translational modifications such as acetylation, phosphorylation, glycosylation, sulfatation, sumoylation, prenylation, ubiquitination, etc.). For further clarity, protein/polypeptide/peptide modifications are envisaged so long as the modification does not destroy the cargo transduction activity of the shuttle agents described herein. For example, shuttle agents described herein may be linear or circular, may be synthesized with one or more D- or L-amino acids, and/or may be conjugated to a fatty acid (e.g., at their N terminus). Shuttle agents described herein may also have at least one amino acid being replaced with a corresponding synthetic amino acid having a side chain of similar physiochemical properties (e.g., structure, hydrophobicity, or charge) as the amino acid being replaced.

As used herein, a “domain” or “protein domain” generally refers to a part of a protein having a particular functionality or function. Some domains conserve their function when separated from the rest of the protein, and thus can be used in a modular fashion. The modular characteristic of many protein domains can provide flexibility in terms of their placement within the shuttle agents of the present description. However, some domains may perform better when engineered at certain positions of the shuttle agent (e.g., at the N- or C-terminal region, or therebetween). The position of the domain within its endogenous protein is sometimes an indicator of where the domain should be engineered within the shuttle agent and of what type/length of linker should be used. Standard recombinant DNA techniques can be used by the skilled person to manipulate the placement and/or number of the domains within the shuttle agents of the present description in view of the present disclosure. Furthermore, assays disclosed herein, as well as others known in the art, can be used to assess the functionality of each of the domains within the context of the shuttle agents (e.g., their ability to facilitate cell penetration across the plasma membrane, endosome escape, and/or access to the cytosol). Standard methods can also be used to assess whether the domains of the shuttle agent affect the activity of the cargo to be delivered intracellularly. In this regard, the expression “operably linked” as used herein refers to the ability of the domains to carry out their intended function(s) (e.g., cell penetration, endosome escape, and/or subcellular targeting) within the context of the shuttle agents of the present description. For greater clarity, the expression “operably linked” is meant to define a functional connection between two or more domains without being limited to a particular order or distance between same.

As used herein, the tem) “synthetic” used in expressions such as “synthetic peptide”, synthetic peptide shuttle agent”, or “synthetic polypeptide” is intended to refer to non-naturally occurring molecules that can be produced in vitro (e.g., synthesized chemically and/or produced using recombinant DNA technology). The purities of various synthetic preparations may be assessed by, for example, high-performance liquid chromatography analysis and mass spectroscopy. Chemical synthesis approaches may be advantageous over cellular expression systems (e.g., yeast or bacteria protein expression systems), as they may preclude the need for extensive recombinant protein purification steps (e.g., required for clinical use). In contrast, longer synthetic polypeptides may be more complicated and/or costly to produce via chemical synthesis approaches and such polypeptides may be more advantageously produced using cellular expression systems. In some embodiments, the peptides or shuttle agents of the present description may be chemically synthesized (e.g., solid- or liquid phase peptide synthesis), as opposed to expressed from a recombinant host cell. In some embodiments, the peptides or shuttle agent of the present description may lack an N-terminal methionine residue. A person of skill in the art may adapt a synthetic peptide or shuttle agent of the present description by using one or more modified amino acids (e.g., non-naturally-occurring amino acids), or by chemically modifying the synthetic peptide or shuttle agent of the present description, to suit particular needs of stability or other needs.

As used herein, the term “independent” is generally intended refer to molecules or agents which are not covalently bound to one another, or that may be transiently covalently linked via a cleavable bond such that the molecules or agents (e.g., shuttle agent and cargo) detach from one another through cleavage of the bond following administration (e.g., when exposed to the reducing cellular environment, and/or but prior to, simultaneously with, or shortly after being delivered intracellularly). For example, the expression “independent cargo” is intended to refer to a cargo to be delivered intracellularly (transduced) that is not covalently bound (e.g., not fused) to a shuttle agent of the present description at the time of transduction across the plasma membrane. In some aspects, having shuttle agents that are independent of (not fused to) a cargo may be advantageous by providing increased shuttle agent versatility—e.g., being able to readily vary the ratio of shuttle agent to cargo (as opposed to being limited to a fixed ratio in the case of a covalent linkage between the shuttle agent and cargo). In some aspects, covalently linking a shuttle agent to its cargo via a cleavable bond such that they detach from one another upon contact with target cells may be advantageous from a manufacturing and/or regulatory perspective.

As used herein, the expression “is or is from” or “is from” comprises functional variants of a given protein or peptide (e.g., a shuttle agent described herein) or domain thereof (e.g., CPD or ELD), such as conservative amino acid substitutions, deletions, modifications, as well as variants or function derivatives, which do not abrogate the activity of the protein domain.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows cargo transduction activity of short/truncated synthetic shuttle agents in HeLa cells for a small molecule cargo, propidium iodide (PI), and a proteinaceous cargo, GFP. The rows are ranked based on “Overall Delivery Factor”, a single calculated number that accounts for the toxicity of each shuttle agent/peptide, as well as its ability to deliver GFP and PI. Structural properties are shown for each peptide, including amino acid sequence, length, hydrophobic moment (μH), helical wheel projection, as well as positively charged and hydrophobic angles. Results are means calculated from experiments performed at least in duplicate.

FIG. 2A shows the inhibitory effect of increasing amounts of sgRNA spiked in the transduction medium on shuttle agent-mediated transduction of a fluorescently-labeled cargo in RH-30 cells evaluated by flow cytometry. The shuttle agent was FSD250 and the cargo was an FITC-labelled phosphorodiamidate morpholino oligomer (PMO-FITC). FIG. 2B shows the results of a transduction assay in which HeLa cells are co-incubated with the shuttle agent FSD250 and GFP as a cargo either in the presence (+) or absence (−) of Cas9-RNP complex, at increasing concentrations of the small positively charged molecule 1,3-diaminoguanidine monohydrochloride as an RNA charge-neutralizing agent. Results are means calculated from experiments performed at least in duplicate.

FIG. 3 shows the results of a transduction assays in which HeLa cells are co-incubated with different peptides/shuttle agents and GFP cargo, in the presence (+) or absence (−) of Cas9-RNP. Results are means calculated from experiments performed at least in duplicate.

FIG. 4A shows the change in GFP transduction efficiency in HeLa cells in the presence (+) or absence (−) of Cas9-RNP for the structurally-related peptides FSD10-15, FSD375, FSD422, FSD424, FSD432, FSD241, FSD231, FSD10, and FSD210.

FIG. 4B shows the change in GFP transduction efficiency in HeLa cells in the presence (+) or absence (−) of Cas9-RNP for the structurally-related peptides CM18, FSD440, CM18-L2-PTD4, His-CM18-Transportan, CM18-TAT, His-CM18-9Arg, and His-CM18-TAT.

FIG. 4C shows the change in GFP transduction efficiency in HeLa cells in the presence (+) or absence (−) of Cas9-RNP for the structurally-related peptides FSD356, FSD357, FSD446, FSD250, FSD296, FSD246, and FSD251.

FIG. 4D shows the change in GFP transduction efficiency in HeLa cells in the presence (+) or absence (−) of Cas9-RNP for the structurally-related peptides FSD374, FSD183, FSD168, FSD172, FSD189, FSD174, and FSD187.

FIG. 5 shows the change in GFP transduction efficiency in CFF-16HBEge cells in the presence (+) or absence (−) of Cas9-RNP for the structurally-related peptides FSD10 and FSD375. FIG. 6A to FIG. 6E each show the ability of structurally different shuttle agents to deliver functional Cpf1-RNP or Cas9-RNP genome editing complexes and effect genome editing in HeLa cells.

FIG. 7 shows the ability of structurally different shuttle agents to deliver functional Cpf1-RNP or Cas9-RNP genome editing complexes and effect genome editing in refractory Human Bronchial Epithelial cell line, CFF-16HBEge.

FIG. 8A to FIG. 8C compare the ability of the shuttle agent FSD10, and variants thereof, to deliver functional Cas9-RNP genome editing complexes versus ABE-Cas9-RNP base editing complexes in CFF-16HBEge cells. FIG. 9 shows the results of a large-scale screening of over 300 candidate peptide shuttle agents for PI and GFP-NLS transduction activity.

FIG. 10 shows core and side view 3D images of the peptides/shuttle agents of FIG. 1 generated by PyMOL. Varying shades of green represent hydrophobic residues (Y, W, I, M, L, F), with darker green representing highly hydrophobic residues; blue residues represent charged hydrophilic residues (K, H, R, E, D); red residues represent uncharged hydrophilic residues (Q, N); and yellow/orange residues represent weakly hydrophobic residues (G, A, S, T).

### SEQUENCE LISTING

This application contains a Sequence Listing in computer readable form created Oct. 21, 2021. The computer readable form is incorporated herein by reference.

| SEQ ID NO: | Description |
|---|---|
| 1 | CM18-Penetratin-cys |
| 2 | TAT-KALA |
| 3 | His-CM18-PTD4 |
| 4 | His-LAH4-PTD4 |
| 5 | PTD4-KALA |
| 6 | EB1-PTD4 |
| 7 | His-CM18-PTD4-6Cys |
| 8 | CM18-PTD4 |
| 9 | CM18-PTD4-6His |
| 10 | His-CM18-PTD4-His |
| 11 | TAT-CM18 |
| 12 | FSD5 |
| 13 | FSD10 |
| 14 | FSD12 |
| 15 | FSD18 |
| 16 | FSD19 |
| 17 | FSD21 |
| 18 | FSD23 |
| 19 | FSD120 |
| 20 | FSD127 |
| 21 | FSD129 |
| 22 | FSD131 |
| 23 | FSD134 |
| 24 | FSD146 |
| 25 | FSD155 |
| 26 | FSD156 |
| 27 | FSD157 |
| 28 | FSD159 |
| 29 | FSD162 |
| 30 | FSD168 |
| 31 | FSD173 |
| 32 | FSD174 |
| 33 | FSD194 |
| 34 | FSD220 |
| 35 | FSD250 |
| 36 | FSD250D |
| 37 | FSD253 |
| 38 | FSD258 |
| 39 | FSD262 |
| 40 | FSD263 |
| 41 | FSD264 |
| 42 | FSD265 |
| 43 | FSD268 |
| 44 | FSD286 |
| 45 | FSD271 |
| 46 | FSD272 |
| 47 | FSD273 |
| 48 | FSD276 |
| 49 | FSD268 Cyclic Amide |
| 50 | FSD268 Cyclic Disulfide |
| 51 | FSD10 Scramble |
| 52 | FSD268 Scramble |
| 53 | FSD174 Scramble |
| 54 | FSN3 |
| 55 | FSN4 |
| 56 | FSN7 |
| 57 | FSN8 |
| 58 | FSD117 |
| 59 | FSD118 |
| 60 | FSD119 |
| 61 | FSD121 |
| 62 | FSD122 |
| 63 | FSD123 |
| 64 | FSD124 |
| 65 | FSD125 |
| 66 | FSD126 |
| 67 | FSD127 |
| 68 | FSD128 |
| 69 | FSD130 |
| 70 | FSD132 |
| 71 | FSD133 |
| 72 | FSD135 |
| 73 | FSD137 |
| 74 | FSD138 |
| 75 | FSD139 |
| 76 | FSD140 |
| 77 | FSD141 |
| 78 | FSD142 |
| 79 | FSD143 |
| 80 | FSD144 |
| 81 | FSD145 |
| 82 | FSD147 |
| 83 | FSD148 |
| 84 | FSD149 |
| 85 | FSD150 |
| 86 | FSD151 |
| 87 | FSD152 |
| 88 | FSD153 |
| 89 | FSD154 |
| 90 | FSD158 |
| 91 | FSD160 |
| 92 | FSD161 |
| 93 | FSD163 |
| 94 | FSD164 |
| 95 | FSD165 |
| 96 | FSD166 |
| 97 | FSD167 |
| 98 | FSD169 |
| 99 | FSD170 |
| 100 | FSD171 |
| 101 | FSD172 |
| 102 | FSD175 |
| 103 | FSD176 |
| 104 | FSD177 |
| 105 | FSD178 |
| 106 | FSD179 |
| 107 | FSD180 |
| 108 | FSD181 |
| 109 | FSD182 |
| 110 | FSD183 |
| 111 | FSD184 |
| 112 | FSD185 |
| 113 | FSD186 |
| 114 | FSD187 |
| 115 | FSD188 |
| 116 | FSD189 |
| 117 | FSD190 |
| 118 | FSD191 |
| 119 | FSD192 |
| 120 | FSD193 |
| 121 | FSD195 |
| 122 | FSD196 |
| 123 | FSD197 |
| 124 | FSD198 |
| 125 | FSD199 |
| 126 | FSD200 |
| 127 | FSD201 |
| 128 | FSD202 |
| 129 | FSD203 |
| 130 | FSD204 |
| 131 | FSD205 |
| 132 | FSD206 |
| 133 | FSD207 |
| 134 | FSD208 |
| 135 | FSD209 |
| 136 | FSD210 |
| 137 | FSD211 |
| 138 | FSD212 |
| 139 | FSD213 |
| 140 | FSD214 |
| 141 | FSD215 |
| 142 | FSD216 |
| 143 | FSD217 |
| 144 | FSD218 |
| 145 | FSD219 |
| 146 | FSD221 |
| 147 | FSD222 |
| 148 | FSD223 |
| 149 | FSD224 |
| 150 | FSD225 |
| 151 | FSD226 |
| 152 | FSD227 |
| 153 | FSD228 |
| 154 | FSD229 |
| 155 | FSD230 |
| 156 | FSD231 |
| 157 | FSD232 |
| 158 | FSD233 |
| 159 | FSD234 |
| 160 | FSD235 |
| 161 | FSD236 |
| 162 | FSD237 |
| 163 | FSD238 |
| 164 | FSD239 |
| 165 | FSD240 |
| 166 | FSD241 |
| 167 | FSD243 |
| 168 | FSD244 |
| 169 | FSD246 |
| 170 | FSD247 |
| 171 | FSD248 |
| 172 | FSD250 Scramble |
| 173 | FSD250E |
| 174 | FSD251 |
| 175 | FSD254 |
| 176 | FSD255 |
| 177 | FSD256 |
| 178 | FSD257 |
| 179 | FSD259 |
| 180 | FSD260 |
| 181 | FSD261 |
| 182 | FSD266 |
| 183 | FSD267 |
| 184 | FSD269 |
| 185 | FSD270 |
| 186 | FSD274 |
| 187 | FSD275 |
| 188 | FSD276 |
| 189 | FSD277 |
| 190 | FSD278 |
| 191 | FSD279 |
| 192 | FSD280 |
| 193 | FSD281 |
| 194 | FSD282 |
| 195 | FSD283 |
| 196 | FSD284 |
| 197 | FSD285 |
| 198 | FSD287 |
| 199 | FSD288 |
| 200 | FSD289 |
| 201 | FSD290 |
| 202 | FSD291 |
| 203 | FSD292 |
| 204 | FSD293 |
| 205 | FSD294 |
| 206 | FSD295 |
| 207 | FSD296 |
| 208 | FSD297 |
| 209 | FSD298 |
| 210 | FSD299 |
| 211 | FSD300 |
| 212 | FSD301 |
| 213 | FSD302 |
| 214 | FSD303 |
| 215 | FSD304 |
| 216 | FSD305 |
| 217 | FSD306 |
| 218 | FSD307 |
| 219 | FSD308 |
| 220 | FSD309 |
| 221 | FSD310 |
| 222 | FSD311 |
| 223 | FSD312 |
| 224 | FSD313 |
| 225 | FSD314 |
| 226 | FSD315 |
| 227 | FSD316 |
| 228 | FSD317 |
| 229 | FSD318 |
| 230 | FSD319 |
| 231 | FSD320 |
| 232 | FSD321 |
| 233 | FSD322 |
| 234 | FSD323 |
| 235 | FSD324 |
| 236 | FSD325 |
| 237 | FSD326 |
| 238 | FSD327 |
| 239 | FSD328 |
| 240 | FSD330 |
| 241 | FSD331 |
| 242 | FSD332 |
| 243 | FSD333 |
| 244 | FSD334 |
| 245 | FSD335 |
| 246 | FSD336 |
| 247 | FSD337 |
| 248 | FSD338 |
| 249 | FSD339 |
| 250 | FSD340 |
| 251 | FSD341 |
| 252 | FSD342 |
| 253 | FSD343 |
| 254 | FSD344 |
| 255 | FSD345 |
| 256 | FSD346 |
| 257 | FSD347 |
| 258 | FSD348 |
| 259 | FSD349 |
| 260 | FSD350 |
| 261 | FSD351 |
| 262 | FSD352 |
| 263 | FSD353 |
| 264 | FSD354 |
| 265 | FSD355 |
| 266 | FSD356 |
| 267 | FSD357 |
| 268 | FSD358 |
| 269 | FSD359 |
| 270 | FSD360 |
| 271 | FSD361 |
| 272 | FSD362 |
| 273 | FSD363 |
| 274 | FSD364 |
| 275 | FSD365 |
| 276 | FSD366 |
| 277 | FSD367 |
| 278 | FSD368 |
| 279 | FSD369 |
| 280 | FSD370 |
| 281 | FSD371 |
| 282 | FSD372 |
| 283 | FSD373 |
| 284 | FSD374 |
| 285 | FSD375 |
| 286 | FSD376 |
| 287 | FSD377 |
| 288 | FSD378 |
| 289 | FSD379 |
| 290 | FSD381 |
| 291 | FSD382 |
| 292 | FSD383 |
| 293 | FSD384 |
| 294 | FSD385 |
| 295 | FSD386 |
| 296 | FSD387 |
| 297 | FSD388 |
| 298 | FSD389 |
| 299 | FSD390 |
| 300 | FSD391 |
| 301 | FSD392 |
| 302 | FSD393 |
| 303 | FSD394 |
| 304 | FSD395 |
| 305 | FSD396 |
| 306 | FSD397 |
| 307 | FSD398 |
| 308 | FSD399 |
| 309 | FSD400 |
| 310 | FSD401 |
| 311 | FSD402 |
| 312 | FSD403 |
| 313 | FSD404 |
| 314 | FSD406 |
| 315 | FSD407 |
| 316 | FSD408 |
| 317 | FSD409 |
| 318 | FSD410 |
| 319 | FSD411 |
| 320 | FSD412 |
| 321 | FSD413 |
| 322 | FSD414 |
| 323 | FSD415 |
| 324 | FSD416 |
| 325 | FSD417 |
| 326 | FSD418 |
| 327 | FSD419 |
| 328 | FSD421 |
| 329 | FSD422 |
| 330 | FSD423 |
| 331 | FSD424 |
| 332 | FSD425 |
| 333 | FSD426 |
| 334 | FSD427 |
| 335 | FSD428 |
| 336 | FSD429 |
| 337 | FSD430 |
| 338 | FSD431 |
| 339 | FSD432 |
| 340 | FSD433 |
| 341 | FSD434 |
| 342 | FSD435 |
| 343 | FSD436 |
| 344 | FSD438 |
| 345 | His-PTD4 |
| 346 | FSD92 |
| 347 | C(LLKK)₃C |
| 348 | CM18 |
| 349 | FSD10-8 |
| 350 | FSD10-12-1 |
| 351 | FSD10-12-2 |
| 352 | FSD10-15 |
| 353 | FSD418-8 |
| 354 | FSD418-12-1 |
| 355 | FSD418-12-2 |
| 356 | FSD418-15 |
| 357 | FSD418-19 |
| 358 | FSD439 |
| 359 | FSD440 |
| 360 | FSD441 |
| 361 | FSD442 |
| 362 | FSD443 |
| 363 | KALA |
| 364 | FSD444 |
| 365 | LAH4 |
| 366 | Penetratin |
| 367 | PTD4 |
| 368 | TAT |
| 369 | FSD445 |
| 370 | FSD446 |
| 371 | FSD447 |
| 372 | crRNA beta-2 microglobulin (B2M) for Cas9 |
| 373 | crRNA beta-2 microglobulin (B2M) for Cpf1 |
| 374 | CM18-L2-PTD4 |
| 375 | His-CM18-Transportan |
| 376 | CM18-TAT |
| 377 | His-CM18-9Arg |
| 378 | His-CM18-TAT |
| 379 | FSD448 |
| 380 | Linker-(FSD10-Cter)-Linker |
| 381 | FSD10-Cter |

### DETAILED DESCRIPTION

In one aspect, described herein are short synthetic peptide shuttle agents having cargo transduction activity and their use for delivering a variety of independent cargoes in eukaryotic cells. As used herein, the expression “short synthetic peptide shuttle agents” or “short shuttle agents” may refer to synthetic peptide shuttle agents less than 20 amino acids in length or may refer to a “core” amphipathic cationic alpha helical region less than 20 amino acids in length within a longer shuttle agent.

In some embodiments, the short shuttle agents generally comprise a helical region comprising an amphipathic helix harboring: a cluster of hydrophobic amino acid residues on one side of the helix defining a hydrophobic angle of 140° to 280° in Schiffer-Edmundson's wheel representation, and a cluster of positively charged residues on the other side of the helix defining a positively charged angle of 40° to 160° in Schiffer-Edmundson's wheel representation. In some embodiments, the cluster of hydrophobic amino acid residues on one side of the helix define a hydrophobic angle of 140° to 280°, 160° to 260°, or 180° to 240° in Schiffer-Edmundson's wheel representation. In some embodiments, the cluster of positively charged residues on the other side of the helix define a positively charged angle of 40° to 160°, 40° to 140°, or 60° to 140° in Schiffer-Edmundson's wheel representation. The foregoing geometries were generally commonly shared by short shuttle agents, as described in Example 3.

In some embodiments, at least 20%, 30%, 40%, or 50% of the residues in the hydrophobic cluster are hydrophobic residues. In some embodiments, the hydrophobic residues are selected from the group consisting of phenylalanine, isoleucine, tryptophan, leucine, valine, methionine, tyrosine, cysteine, glycine, and alanine. In some embodiments, the hydrophobic residues are selected from the group consisting of phenylalanine, isoleucine, tryptophan, and/or leucine.

In some embodiments, at least 20%, 30%, 40%, or 50% of the residues in the positively charged cluster are positively charged residues. In some embodiments, the positively charged residues are selected from the group consisting of lysine, arginine, and histidine. In some embodiments, the positively charged residues are selected from the group consisting of lysine and arginine.

In some embodiments, the short synthetic peptide shuttle agent is at least 8, 9, 10, 11, 12, 13, 14, 16, 17, 18, or 19 amino acids in length.

In some embodiments, the short synthetic peptide shuttle agent may have a hydrophobic moment (μH) of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.1, 5.2, 5.3, 5.4, or 5.5. Preferably, the short shuttle agents have a hydrophobic moment of at least 3.5, 4, or 4.5.

In some embodiments, the short shuttle agents may be used for transducing cargoes such as polypeptides, peptides, nucleoproteins, small molecules, or oligonucleotide analogs (e.g., non-anionic oligonucleotide analogs).

In some aspects, described herein are compositions and methods for nucleoprotein cargo transduction. The compositions generally comprise a nucleoprotein cargo for intracellular delivery and a synthetic peptide shuttle agent that is independent from, or is not covalently linked to, said nucleoprotein cargo. The synthetic peptide shuttle agent is a peptide comprising an amphipathic alpha-helical motif having both a positively-charged hydrophilic outer face and a hydrophobic outer face, wherein synthetic peptide shuttle agent increases cytosolic/nuclear delivery of said nucleoprotein cargo in eukaryotic cells as compared to in the absence of the synthetic peptide shuttle agent.

In some embodiments, the nucleoprotein cargo may be a deoxyribonucleoprotein (DNP) and/or ribonucleoprotein (RNP) complex. In some embodiments, the nucleoprotein cargo may be an RNA-guided nuclease, a Cas nuclease, such as a Cas type I, II, III, IV, V, or VI nuclease, or a variant thereof that lacking nuclease activity, a base editor, a CRISPR-associated transposase, a Cas-recombinase (e.g., recCas9), or a Cas prime editor. In some embodiments, the nucleoprotein cargo may be Cpf1-RNP (Cas12a-RNP) or Cas9-RNP. In some embodiments, the nucleoprotein cargo comprises a polynucleotide from 10 to 50 bases, 50 to 75 bases, 50 to 100 bases, 50 to 150 bases, 50 to 200 bases, 50 to 250 bases, 75 to 150 bases, or 75 to 125 bases.

In some embodiments, the nucleoprotein cargo is not covalently linked or pre-complexed with a cell-penetrating or cationic peptide. In some embodiments, the nucleoprotein cargo is not encapsulated or combined with a lipid-based carrier.

### Rational Design Parameters and Peptide Shuttle Agents

In some aspects, the shuttle agents described herein may be a peptide having transduction activity for nucleoprotein cargoes, proteinaceous cargoes, small molecules, non-anionic polynucleotide analogs, or any combination thereof, in target eukaryotic cells (WO/2018/068135, CA 3,040,645, WO/2020/210916, PCT/CA2021/051458).

In some embodiments, the shuttle agents described herein preferably satisfy one or more or any combination of the following fifteen rational design parameters.

(1) In some embodiments, the shuttle agent is a peptide at least 12, 13, 14, 15, 16, 17, 18, 19, or 20 amino acids in length. For example, the peptide may comprise a minimum length of 17, 18, 19, 20, 21, 22, 23, 24, 25, 26, 27, 28, 29, or 30 amino acid residues, and a maximum length of 35, 36, 37, 38, 39, 40, 41, 42, 43, 44, 45, 46, 47, 48, 49, 50, 51, 52, 53, 54, 55, 60, 65, 70, 75, 80, 85, 90, 95, 100, 105, 110, 115, 120, 125, 130, 135, 140, 145, or 150 amino acid residues. In some embodiments, shorter peptides (e.g., in the 17-50 or 20-50 amino acid range) may be particularly advantageous because they may be more easily synthesized and purified by chemical synthesis approaches, which may be more suitable for clinical use (as opposed to recombinant proteins that must be purified from cellular expression systems). While numbers and ranges in the present description are often listed as multiples of 5, the present description should not be so limited. For example, the maximum length described herein should be understood as also encompassing a length of 56, 57, 58 . . . 61, 62, etc., in the present description, and that their non-listing herein is only for the sake of brevity. The same reasoning applies to the % of identities listed herein.

(2) In some embodiments, the peptide shuttle agent comprises an amphipathic alpha-helical motif at neutral pH. As used herein, the expression “alpha-helical motif” or “alpha-helix”, unless otherwise specified, refers to a right-handed coiled or spiral conformation (helix) having angle of rotation between consecutive amino acids of 100 degrees and/or an alpha-helix having 3.6 residues per turn. As used herein, the expression “comprises an alpha-helical motif” or “an amphipathic alpha-helical motif” and the like, refers to the three-dimensional conformation that a peptide (or segment of a peptide) of the present description is predicted to adopt when in a biological setting at neutral pH based on the peptide's primary amino acid sequence, regardless of whether the peptide actually adopts that conformation when used in cells as a shuttle agent. Furthermore, the peptides of the present description may comprise one or more alpha-helical motifs in different locations of the peptide. For example, the shuttle agent FSD5 in WO/2018/068135 is predicted to adopt an alpha-helix over the entirety of its length (see FIG. 49C of WO/2018/068135), while the shuttle agent FSD18 of WO/2018/068135 is predicted to comprise two separate alpha-helices towards the N and C terminal regions of the peptide (see FIG. 49D of WO/2018/068135). In some embodiments, the shuttle agents of the present description are not predicted to comprise a beta-sheet motif, for example as shown in FIGS. 49E and 49F of WO/2018/068135. Methods of predicting the presence of alpha-helices and beta-sheets in proteins and peptides are well known in the art. For example, one such method is based on 3D modeling using PEP-FOLD™, an online resource for de novo peptide structure prediction (http://bioserv.ipbs.univ-paris-diderot.fr/services/PEP-FOLD/) (Lamiable et al., 2016; Shen et al., 2014; Thévenet et al., 2012). Other methods of predicting the presence of alpha-helices in peptides and protein are known and readily available to the skilled person.

As used herein, the expression “amphipathic” refers to a peptide that possesses both hydrophobic and hydrophilic elements (e.g., based on the side chains of the amino acids that comprise the peptide). For example, the expression “amphipathic alpha helix” or “amphipathic alpha-helical motif” refers to a peptide predicted to adopt an alpha-helical motif having a non-polar hydrophobic face and a polar hydrophilic face, based on the properties of the side chains of the amino acids that form the helix.

(3) In some embodiments, peptide shuttle agents of the present description comprise an amphipathic alpha-helical motif having a positively-charged hydrophilic outer face, such as one that is rich in R and/or K residues. As used herein, the expression “positively-charged hydrophilic outer face” refers to the presence of at least three lysine (K) and/or arginine (R) residues clustered to one side of the amphipathic alpha-helical motif, based on alpha-helical wheel projection (e.g., see FIG. 49A, left panel of WO/2018/068135). Such helical wheel projections may be prepared using a variety of programs, such as the online helical wheel projection tool created by Don Armstrong and Raphael Zidovetzki. (e.g., available at: https://www.donarmstrong.com/cgi-bin/wheel.pl) or the online tool developed by Mól et al., 2018 (e.g., available at http://lbqp.unb.br/NetWheels/). In some embodiments, the amphipathic alpha-helical motif may comprise a positively-charged hydrophilic outer face that comprises: (a) at least two, three, or four adjacent positively-charged K and/or R residues upon helical wheel projection; and/or (b) a segment of six adjacent residues comprising three to five K and/or R residues upon helical wheel projection, based on an alpha helix having angle of rotation between consecutive amino acids of 100 degrees and/or an alpha-helix having 3.6 residues per turn.

In some embodiments, peptide shuttle agents of the present description comprise an amphipathic alpha-helical motif comprising a hydrophobic outer face, the hydrophobic outer face comprising: (a) at least two adjacent L residues upon helical wheel projection; and/or (b) a segment often adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn.

(4) In some embodiments, peptide shuttle agents of the present description comprise an amphipathic alpha-helical motif having a highly hydrophobic core composed of spatially adjacent highly hydrophobic residues (e.g., L, I, F, V, W, and/or M). In some embodiments, the highly hydrophobic core may consist of spatially adjacent L, I, F, V, W, and/or M amino acids representing 12 to 50% of the amino acids of the peptide, calculated while excluding any histidine-rich domains (see below), based on an open cylindrical representation of the alpha-helix having 3.6 residues per turn, as shown for example in FIG. 49A, right panel of WO/2018/068135. In some embodiments, the highly hydrophobic core may consist of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the peptide. More particularly, highly hydrophobic core parameter may be calculated by first arranging the amino acids of the peptide in an opened cylindrical representation, and then delineating an area of contiguous highly hydrophobic residues (L, I, F, V, W, M), as shown in FIG. 49A, right panel of WO/2018/068135. The number of highly hydrophobic residues comprised in this delineated highly hydrophobic core is then divided by the total amino acid length of the peptide, excluding any histidine-rich domains (e.g., N- and/or C-terminal histidine-rich domains). For example, for the peptide shown in FIG. 49A of WO/2018/068135, there are 8 residues in the delineated highly hydrophobic core, and 25 total residues in the peptide (excluding the terminal 12 histidines). Thus, the highly hydrophobic core is 32% (8/25).

(5) Hydrophobic moment relates to a measure of the amphiphilicity of a helix, peptide, or part thereof, calculated from the vector sum of the hydrophobicities of the side chains of the amino acids (Eisenberg et al., 1982). An online tool for calculating the hydrophobic moment of a polypeptide is available from: http://rzlab.ucr.edu/scripts/wheel/wheel.cgi. A high hydrophobic moment indicates strong amphiphilicity, while a low hydrophobic moment indicates poor amphiphilicity. In some embodiments, peptide shuttle agents of the present description may consist of or comprise a peptide or alpha-helical domain having have a hydrophobic moment (μ) of 3.5 to 11. In some embodiments, the shuttle agent may be a peptide comprising an amphipathic alpha-helical motif having a hydrophobic moment between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0. In some embodiments, the shuttle agent may be a peptide having a hydrophobic moment between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.2, 10.3, 10.4, or 10.5. In some embodiments, the hydrophobic moment is calculated excluding any histidine-rich domains that may be present in the peptide.

(6) In some embodiments, peptide shuttle agents of the present description may have a predicted net charge of at least +3 or +4 at physiological pH, calculated from the side chains of K, R, D, and E residues. For example, the net charge of the peptide may be at least +5, +6, +7, at least +8, at least +9, at least +10, at least +11, at least +12, at least +13, at least +14, or at least +15 at physiological pH. These positive charges are generally conferred by the greater presence of positively-charged lysine and/or arginine residues, as opposed to negatively charged aspartate and/or glutamate residues.

(7) In some embodiments, peptide shuttle agents of the present description may have a predicted isoelectric point (pI) of 8 to 13, preferably from 10 to 13. Programs and methods for calculating and/or measuring the isoelectric point of a peptide or protein are known in the art. For example, pI may be calculated using the Prot Param software available at: http://web.expasy.org/protparam/

(8) In some embodiments, peptide shuttle agents of the present description may be composed of 35 to 65% of hydrophobic residues (A, C, G, I, L, M, F, P, W, Y, V). In particular embodiments, the peptide shuttle agents may be composed of 36% to 64%, 37% to 63%, 38% to 62%, 39% to 61%, or 40% to 60% of any combination of the amino acids: A, C, G, I, L, M, F, P, W, Y, and V.

(9) In some embodiments, peptide shuttle agents of the present description may be composed of 0 to 30% of neutral hydrophilic residues (N, Q, S, T). In particular embodiments, the peptide shuttle agents may be composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T.

(10) In some embodiments, peptide shuttle agents of the present description may be composed of 35 to 85% of the amino acids A, L, K and/or R. In particular embodiments, the peptide shuttle agents may be composed of 36% to 80%, 37% to 75%, 38% to 70%, 39% to 65%, or 40% to 60% of any combination of the amino acids: A, L, K, or R.

(11) In some embodiments, peptide shuttle agents of the present description may be composed of 15 to 45% of the amino acids A and/or L, provided there being at least 5% of L in the peptide. In particular embodiments, the peptide shuttle agents may be composed of 15% to 40%, 20% to 40%, 20 to 35%, or 20 to 30% of any combination of the amino acids: A and L, provided there being at least 5% of L in the peptide.

(12) In some embodiments, peptide shuttle agents of the present description may be composed of 20 to 45% of the amino acids K and/or R. In particular embodiments, the peptide shuttle agents may be composed of 20% to 40%, 20 to 35%, or 20 to 30% of any combination of the amino acids: K and R.

(13) In some embodiments, peptide shuttle agents of the present description may be composed of 0 to 10% of the amino acids D and/or E. In particular embodiments, the peptide shuttle agents may be composed of 5 to 10% of any combination of the amino acids: D and E.

(14) In some embodiments, the absolute difference between the percentage of A and/or L and the percentage of K and/or R in the peptide shuttle agent may be less than or equal to 10%. In particular embodiments, the absolute difference between the percentage of A and/or L and the percentage of K and/or R in the peptide shuttle agent may be less than or equal to 9%, 8%, 7%, 6%, or 5%.

(15) In some embodiments, peptide shuttle agents of the present description may be composed of 10% to 45% of the amino acids Q, Y, W, P, I, S, G, V, F, E, D, C, M, N, T, or H (i.e., not A, L, K, or R). In particular embodiments, the peptide shuttle agents may be composed of 15 to 40%, 20% to 35%, or 20% to 30% of any combination of the amino acids: Q, Y, W, P, I, S, G, V, F, E, D, C, M, N, T, and H.

In some embodiments, peptide shuttle agents of the present description respect at least one, at least two, at least three, at least four, at least five, at least six, at least seven, at least eight, at least nine, at least ten, at least eleven, at least twelve, at leave thirteen, at least fourteen, or all of parameters (1) to (15) described herein. In particular embodiments, peptide shuttle agents of the present description respect all of parameters (1) to (3), and at least six, at least seven, at least eight, at least nine, at least ten, at least eleven, or all of parameters (4) to (15) described herein.

In some embodiments, where a peptide shuttle agent of the present description comprises only one histidine-rich domain, the residues of the one histidine-rich domain may be included in the calculation/assessment of parameters (1) to (15) described herein. In some embodiments, where a peptide shuttle agent of the present description comprises more than one histidine-rich domain, only the residues of one of the histidine-rich domains may be included in the calculation/assessment of parameters (1) to (15) described herein. For example, where a peptide shuttle agent of the present description comprises two histidine-rich domains: a first histidine-rich domain towards the N terminus, and a second histidine-rich domain towards the C terminus, only the first histidine-rich domain may be included in the calculation/assessment of parameters (1) to (15) described herein.

In some embodiments, a machine-learning or computer-assisted design approach may be implemented to generate peptides that respect one or more of parameters (1) to (15) described herein. Some parameters, such as parameters (1) and (5)-(15), may be more amenable to implementation in a computer-assisted design approach, while structural parameters, such as parameters (2), (3) and (4), may be more amenable to a manual design approach. Thus, in some embodiments, peptides that respect one or more of parameters (1) to (15) may be generated by combining computer-assisted and manual design approaches. For example, multiple sequence alignment analyses of a plurality of peptides shown herein (and others) to function as effective shuttle agents revealed the presence of some consensus sequences—i.e., commonly found patterns of alternance of hydrophobic, cationic, hydrophilic, alanine and glycine amino acids. The presence of these consensus sequences are likely to give rise to structural parameters (2), (3) and (4) being respected (i.e., amphipathic alpha-helix formation, a positively-charged face, and a highly hydrophobic core of 12%-50%). Thus, these and other consensus sequences may be employed in machine-learning and/or computer-assisted design approaches to generate peptides that respect one or of parameters (1)-(15).

Accordingly, in some embodiments, peptide shuttle agents described herein may comprise or consist of the amino acid sequence of:

(a)[X1]-[X2]-[linked]-[X3]-[X4] (Formula 1);

(b)[X1]-[X2]-[linked]-[X4]-[X3] (Formula 2);

(c)[X2]-[X1]-[linked]-[X3]-[X4] (Formula 3);

(d)[X2]-[X1]-[linked]-[X4]-[X3] (Formula 4);

(e)[X3]-[X4]-[linked]-[X1]-[X2] (Formula 5);

(f)[X3]-[X4]-[X1] (Formula 6);

(g)[X4]-[X3]-[linked]-[X1]-[X2] (Formula 7);

(h)[X4]-[X3]-[X1] (Formula 8);

(i)[linker]-[X1]-[X2]-[linker] (Formula 9);

(j)[linker]-[X2]-[X1]-[linker] (Formula 10);

(k)[X1]-[X2]-[linker] (Formula 11);

(l)[X2]-[X1]-[linker] (Formula 12);

(m)[linker]-[X1]-[X2] (Formula 13);

(n)[linker]-[X2]-[X1] (Formula 14);

(o)[X1]-[X2] (Formula 15); or

(p)[X2]-[X1] (Formula 16),

wherein:
[X1] is selected from: 2[Φ]-1[+]-2[Φ]-1[ζ]-1[+]- ; 2[Φ]-1[+]-2[Φ]-2[+]- ; 1[+]- 1[Φ]-1[+]-2[Φ]-1[ζ]-1[+]- ; and 1 [+]-1[Φ]-1[+]-2[Φ]-2 [+]- ;
[X2] is selected from: -2[Φ]-1[+]-2[Φ]-2[ζ]- ; -2 [Φ]- 1[+]-2[Φ]-2 [+]- ; -2[Φ]-1 [+]-2[Φ]-1[+]-1[ζ]-; -2 [Φ]-1 [+]-2[Φ]-1[ζ]-1 [+]- ; -2[Φ]-2[+]-1 [Φ]-2 [+]- ; -2 [Φ]-2 [+]-1 [Φ]-2[ζ]- ; -2[Φ]-2 [+]-1[Φ]-1[+]-1[ζ]- ; and -2 [Φ]-2[+]-1[Φ]-1[ζ]-1[+]- ;
[X3] is selected from: -4[+]-A- ; -3[+]-G-A- ; -3[+]-A-A- ; -2[+]-1[Φ]-1 [+]-A- ; -2[+]-1[Φ]-G-A- ; -2[+]-1[Φ]-A-A- ; or -2[+]-A-1[+]-A; -2[+]-A-G-A; -2[+]-A-A-A- ; -1 [Φ]-3[+]-A- ; -1 [Φ]-2[+]-G-A- ; -1[Φ]-2[+]-A-A- ; -1 [Φ]-1 [+]-1[Φ]-1[+]-A; -1 [Φ]-1 [+]-1[Φ]-G-A; -1[Φ]-1[+]-1 [Φ]-A-A; -1[Φ]-1[+]-A-1 [+]-A; -1 [Φ]-1 [+]-A-G-A; -1 [Φ]-1 [+]-A-A-A; -A-1 [+]-A-1 [+]-A; -A-1[+]-A-G-A; and -A-1[+]-A-A-A;
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1 [ζ]-A-1 [ζ]-A-1[+]; -2 [+]-A-2[+]; -2 [+]-A-1 [+]-A; -2 [+]-A-1[+]-1[Φ]-A-1[+]; - 2[+]-1[ζ]-A-1[+]; -1[+]-1[ζ]-A-1[+]-A ; -1[+]-1[ζ]-A-2[+]; -1 [+]-1[ζ]-A-1[+]-1 [ζ]-A-1[+]; -1[+]-2 [ζ]-A-1 [+]; -1[+]-2[ζ]-2[+]; -1[+]-2[ζ]-1 [+]-A; -1 [+]-2[ζ]-1[+]-1[ζ]-A-1 [+]; -1[+]-2 [ζ]-1 [ζ]-A-1[+]; -3[ζ]-2[+]; -3[ζ]-1[+]-A; -3[ζ]-1[+]-1[ζ]-A-1[+]; -1[ζ]-2A-1[+]-A; -1[ζ]-2A-2[+]; -1[ζ]-2A-1[+]-1[ζ]-A-1 [+]; -2[+]-A-1[+]-A; -2[+]-1[ζ]-1[+]-A; -1[+]-1[ζ]-A-1[+]-A; -1 [+]-2A-1[+]-1[ζ]-A-1 [+]; and -1[ζ]-A-1[ζ]-A-1[+]; and
[linker] is selected from: -Gn- ; —Sn—; -(GnSn)n- ; -(GnSn)nGn- ; -(GnSn)nSn—; -(GnSn)nGn(GnSn)n-; and -(GnSn)nSn(GnSn)n- ;
   wherein: [Φ] is an amino acid which is: Leu, Phe, Trp, Ile, Met, Tyr, or Val, preferably Leu, Phe, Trp, or Ile; [+] is an amino acid which is: Lys or Arg; [ζ] is an amino acid which is: Gln, Asn, Thr, or Ser; A is the amino acid Ala; G is the amino acid Gly; S is the amino acid Ser; and n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4, or 1 to 3.

In some embodiments, peptide shuttle agents of the present description may comprise or consist of a peptide which is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370, or 379 or to the amino acid sequence of any one of SEQ ID NOs: 104, 105, 107, 108, 110-131, 133-135, 138, 140, 142, 145, 148, 151, 152, 169-242, and 243-10 242 as disclosed in WO/2018/068135, or a functional variant thereof. In some embodiments, peptide shuttle agents of the present description may comprise the amino acid sequence motifs of SEQ ID NOs: 158 and/or 159 of WO/2018/068135, which were found in each of peptides FSD5, FSD16, FSD18, FSD19, FSD20, FSD22, and FSD23. In some embodiments, peptide shuttle agents of the present description may comprise the amino acid sequence motif of SEQ ID NO: 158 of WO/2018/068135 operably linked to the amino acid sequence motif of SEQ ID NO: 159 of WO/2018/068135. As used herein, a “functional variant” refers to a peptide having cargo transduction activity, which differs from the reference peptide by one or more conservative amino acid substitutions. As used herein in the context of functional variants, a “conservative amino acid substitution” is one in which one amino acid residue is replaced with another amino acid residue having a similar side chain Families of amino acid residues having similar side chains have been well defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, phenylalanine, methionine, tryptophan, and optionally proline), beta-branched side chains (e.g., threonine, valine, isoleucine) and aromatic side chains (e.g., tyrosine, phenylalanine, tryptophan, histidine).

In some embodiments, peptide shuttle agents of the present description do not comprise one or more of the amino acid sequences of any one of SEQ ID NOs: 57-59, 66-72, or 82-102 of WO/2018/068135. In some embodiments, peptide shuttle agents of the present description do not comprise one or more of the amino acid sequences of any one of SEQ ID NOs: 104, 105, 107, 108, 110-131, 133-135, 138, 140, 142, 145, 148, 151, 152, 169-242, and 243-10 242 as disclosed in WO/2018/068135. Rather, in some embodiments, peptide shuttle agents of the present description may relate to variants of such previously described shuttle agent peptides, wherein the variants are further engineered for improved transduction activity (i.e., capable of more robustly transducing nucleoprotein cargoes).

In some embodiments, peptide shuttle agents of the present description may have a minimal threshold of transduction efficiency and/or cargo delivery score for a “surrogate” cargo as measured in a eukaryotic cell model system (e.g., an immortalized eukaryotic cell line) or in a model organism. The expression “transduction efficiency” refers to the percentage or proportion of a population of target cells into which a cargo of interest is delivered intracellularly, which can be determined for example by flow cytometry, immunofluorescence microscopy, and other suitable methods may be used to assess cargo transduction efficiency (e.g., as described in WO/2018/068135). In some embodiments, transduction efficiency may be expressed as a percentage of cargo-positive cells. In some embodiments, transduction efficiency may be expressed as a fold-increase (or fold-decrease) over a suitable negative control assessed under identical conditions except for in the absence of cargo and shuttle agent (“no treatment”; NT) or in the absence of shuttle agent (“cargo alone”).

In some embodiments, the shuttle agents described herein comprises or consists of:
(i) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370, or 379;
(ii) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370, or 379 by no more than 1, 2, 3, 4, 5, 6, 7, 8, 9, or 10 amino acids (e.g., excluding any linker domains);
(iii) an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370, or 379 (e.g., calculated excluding any linker domains or glycine/serine-rich flanking domains);
(iv) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370, or 379 by only conservative amino acid substitutions (e.g., by no more than no more than 1, 2, 3, 4, 5, 6, 7, 8, 9, or 10 conservative amino acid substitutions, preferably excluding any linker domains or glycine/serine-rich flanking domains), wherein each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and their amides: D, E, N, and Q; or
(v) any combination of (i) to (iv).

In some embodiments, shuttle agents described herein are preferably second generation shuttle agents lacking a cell-penetrating domain or lack a cell-penetrating domain fused to an endosome leakage domain. In some embodiments, shuttle agents described herein particularly suitable for delivery of nucleoprotein cargoes are preferably those having relatively high transduction efficiencies over high delivery scores, meaning that the shuttle agents deliver cargo to a greater percentage of cells (instead of a greater total number of cargo molecules per cell). Indeed, excess CRISPR-Cas genome editing complexes delivered intracellularly may increase the probability of off-target effects. In some embodiments, shuttle agents described herein (and/or the SEQ ID NOs recited above in the preceding paragraph) are those listed in FIG. 9 having a Mean % of PI+ cells or a Mean % of GFP+ cells of at least 20, 25, 30, 35, 40, 45, 50, 55, 60, 65, 70, or 75%.

In some embodiments, the shuttle agents described herein comprise or consist of a variant of the synthetic peptide shuttle agent, the variant being identical to the synthetic peptide shuttle agent as defined herein, except having at least one amino acid being replaced with a corresponding synthetic amino acid having a side chain of similar physiochemical properties (e.g., structure, hydrophobicity, or charge) as the amino acid being replaced, wherein the variant increases cytosolic/nuclear delivery of said cargo in eukaryotic cells as compared to in the absence of the synthetic peptide shuttle agent.

In some embodiments, the shuttle agents described herein may comprise or consist of a fragment of a longer parent shuttle agent as described or referred to herein, wherein the fragment retains cargo transduction activity and comprises an amphipathic alpha-helical motif having both a positively-charged hydrophilic outer face and a hydrophobic outer face. In some embodiments, the shuttle agents described herein may comprise or consist of a variant of a parent shuttle agent as described or referred to herein, wherein the variant retains cargo transduction activity and differs (or differs only) from the parent shuttle agent by having a reduced N-terminal and/or C-terminal positive charge density relative to the parent shuttle agent. As used herein “positive charge density” refers to the total number of residues with positively charged sidechains at physiological pH per length of the peptide. For example, three consecutive arginine residues (RRR) have a greater charge density than three arginine residues spaced farther apart by non-cationic residues (e.g., RARAR). In some embodiments, positive charge density may be reduced by substituting one or more cationic residues, such as K/R, with non-cationic residues, preferably non-cationic hydrophilic residues; and/or by engineering hydrophobic residues (e.g., A, V, L, I, F, or W) between two proximal cationic residues. In some embodiments, positive charge density may be reduced by increasing the distance between positive charge residues in close proximity in the peptide. In some embodiments, the shuttle peptide fragments or variants described herein, or the short shuttle agents described herein, preferably have increased resistance to inhibition by the nucleoprotein cargo, and/or has increased transduction activity for the nucleoprotein cargo. In some embodiments, shuttle peptide fragments or variants described herein, or the short shuttle agents described herein may comprise or consist of a C-terminal truncation of a longer parent shuttle agent.

In some embodiments, shuttle peptide fragments or variants described herein, or the short shuttle agents described herein, may comprise a “core” amphipathic alpha-helical motif having both a positively-charged hydrophilic outer face and a hydrophobic outer face, which is flanked by or at least by 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 non-cationic hydrophilic residues, such that the fragment or variant retains cargo transduction activity and/or has increased resistance to inhibition by the nucleoprotein cargo or by the presence of extracellular DNA/RNA.

### Chemical Modifications and Synthetic Amino Acids

In some embodiments, shuttle agents of the present description may comprise oligomers (e.g., dimers, trimers, etc.) of peptides described herein. Such oligomers may be constructed by covalently binding the same or different types of shuttle agent monomers (e.g., using disulfide bridges to link cysteine residues introduced into the monomer sequences). In some embodiments, shuttle agents of the present description may comprise an N-terminal and/or a C-terminal cysteine residue.

In some embodiments, shuttle agents of the present description may comprise or consist of a cyclic peptide. In some embodiments, the cyclic peptide may be formed via a covalent link between a first residue positioned towards the N terminus of the shuttle agent and a second residue positioned towards the C terminus of the shuttle agent. In some embodiments, the first and second residues are flanking residues positioned at the N and the C termini of the shuttle agent. In some embodiments, the first and second residues may be linked via an amide linkage to form the cyclic peptide. In some embodiments, the cyclic peptide may be formed by a disulfide bond between two cysteine residues within the shuttle agent, wherein the two cysteine residues are positioned towards the N and C termini of the shuttle agent. In some embodiments, the shuttle agent may comprise, or be engineered to comprise, flanking cysteine residues at the N and C termini, which are linked via a disulfide bond to form the cyclic peptide. In some embodiments, the cyclic shuttle agents described herein may be more resistant to degradation (e.g., by proteases) and/or may have a longer half-life than a corresponding linear peptide.

In some embodiments, the shuttle agents of the present description may comprise one or more D-amino acids. In some embodiments, the shuttle agents of the present description may comprise a D-amino acid at the N and/or C terminus of the shuttle agent. In some embodiments, the shuttle agents maybe comprised entirely of D-amino acids. In some embodiments, the shuttle agents described herein having one or more D-amino acids may be more resistant to degradation (e.g., by proteases) and/or may have a longer half-life than a corresponding peptide comprised of only L-amino acids.

In some embodiments, the shuttle agents of the present description may comprise a chemical modification to one or more amino acids, wherein the chemical modification does not destroy the transduction activity of the synthetic peptide shuttle agent. As used herein in this context, the term “destroy” means that the chemical modification irreversibly abolishes the cargo transduction activity of a peptide shuttle agent described herein. Chemical modifications that may transiently inhibit, attenuate, or delay the cargo transduction activity of a peptide shuttle agent described herein may be included in the chemical modifications to the shuttle agents of the present description. In some embodiments, the chemical modification to any one of the shuttle agents described herein may be at the N and/or C terminus of the shuttle agent. Examples of chemical modifications include the addition of an acetyl group (e.g., an N-terminal acetyl group), a cysteamide group (e.g., a C-terminal cysteamide group), or a fatty acid (e.g., C4-C16, C6-C14, C6-C12, C6-C8, or C8 fatty acid, preferably being N-terminal).

In some embodiments, the shuttle agents of the present description comprise shuttle agent variants having cargo transduction activity in target eukaryotic cells, the variants being identical to any shuttle agent of the present description, except having at least one amino acid being replaced with a corresponding synthetic amino acid or amino acid analog having a side chain of similar physiochemical properties (e.g., structure, hydrophobicity, or charge) as the amino acid being replaced. In some embodiments, the synthetic amino acid replacement:
(a) replaces a basic amino acids with any one of: α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine , β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β,β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid; and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine

### Histidine-Rich Domains

In some embodiments, shuttle agents of the present description may further comprise one or more histidine-rich domains. In some embodiments, the histidine-rich domain may be a stretch of at least 2, at least 3, at least 4, at least 5, or at least 6 amino acids comprising at least 30%, at least 35%, at least 40%, at least 45%, at least 50%, at least 55%, at least 60%, at least 65%, at least 70%, at least 75%, at least 80%, at least 85%, or at least 90% histidine residues. In some embodiments, the histidine-rich domain may comprise at least 2, at least 3, at least 4 at least 5, at least 6, at least 7, at least 8, or at least 9 consecutive histidine residues. Without being bound by theory, in the context of first generation shuttle agents comprising a CPD operably linked to an ELD, the histidine-rich domain in the shuttle agent may act as a proton sponge in the endosome through protonation of their imidazole groups under acidic conditions of the endosomes, providing another mechanism of endosomal membrane destabilization and thus further facilitating the ability of endosomally-trapped cargoes to gain access to the cytosol. In some embodiments, the histidine-rich domain may be located at or towards the N and/or C terminus of the peptide shuttle agent.

### Linkers

In some embodiments, peptide shuttle agents of the present description may comprise one or more suitable linkers (e.g., flexible polypeptide linkers). In some embodiments, such linkers may separate two or more amphipathic alpha-helical motifs (e.g., see the shuttle agent FSD18 in FIG. 49D of WO/2018/068135), or a core amphipathic cationic motif from another motif. In some embodiments, linkers can be used to separate two more domains (CPDs, ELDs, or histidine-rich domains) from one another. In some embodiments, linkers may be formed by adding sequences of small hydrophobic amino acids with or without rotatory potential (such as glycine) and polar serine residues that confer stability and flexibility. Linkers may be soft and allow the domains of the shuttle agents to move. In some embodiments, prolines may be avoided since they can add significant conformational rigidity. In some embodiments, the linkers may be serine/glycine-rich linkers. In some embodiments, the use shuttle agents comprising a suitable linker may be advantageous for delivering a cargo to suspension cells, rather than to adherent cells. In some embodiments, the linker may comprise or consist of: -Gn- ; —Sn—; -(GnSn)n- ; -(GnSn)nGn- ; -(GnSn)nSn—; -(GnSn)nGn(GnSn)n- ;

or -(GnSn)nSn(GnSn)n- , wherein G is the amino acid Gly; S is the amino acid Ser; and n is an integer from 1 to 5. In some embodiments, short stretches or “linkers” of flexible and/or hydrophilic amino acids (e.g., glycine/serine-rich stretches) may be added to the N terminus, C terminus, or both the N and C termini of a shuttle agent or core alpha helical amphipathic cationic domain described herein, or a C-terminal truncated shuttle agent described herein. In some embodiments, such stretches may facilitate dissolution of shuttle agents, particularly shorter shuttle agents (e.g., having an amphipathic alpha helical structure with a strongly hydrophobic portion) that would otherwise be insoluble or only partially soluble in aqueous solution. In some embodiments, increasing the solubility of shuttle agent peptides may avoid the use of organic solvents (e.g., DMSO) that may obscure cargo transduction results and/or make the shuttle agents incompatible for therapeutic applications. In some embodiments, the presence of flexible linkers flanking a central core alpha helical amphipathic cationic domain may provide enhanced resistance of the shuttle agent to inhibition by nucleoproteins and/or extracellular DNA/RNA.

### Domain-Based Peptide Shuttle Agents

In some aspects, the shuttle agents described herein may be a first generation shuttle agent as described in WO/2016/161516, comprising an endosome leakage domain (ELD) operably linked to a cell penetrating domain (CPD).

### Endosome Leakage Domains (ELDs)

In some aspects, peptide shuttle agents of the present description may comprise an endosome leakage domain (ELD) having endosomolytic activity. As used herein, the expression “endosome leakage domain” refers to a sequence of amino acids which confers the ability of endosomally-trapped cargoes to gain access to the cytoplasmic compartment. Without being bound by theory, endosome leakage domains are short sequences (often derived from viral or bacterial peptides), which are believed to induce destabilization of the endosomal membrane and liberation of the endosome contents into the cytoplasm. As used herein, the expression “endosomolytic” or “endosomolytic peptide” is intended to refer to this general class of peptides having endosomal membrane-destabilizing properties. Accordingly, in some embodiments, synthetic peptide or polypeptide-based shuttle agents of the present description may comprise an ELD which is an endosomolytic peptide. The activity of such peptides may be assessed for example using the calcein endosome escape assays described in Example 2 of WO/2016/161516.

In some embodiments, the ELD may be a peptide that disrupts membranes at acidic pH, such as pH-dependent membrane active peptide (PMAP) or a pH-dependent lytic peptide. For example, the peptides GALA and INF-7 are amphiphilic peptides that form alpha helixes when a drop in pH modifies the charge of the amino acids which they contain. More particularly, without being bound by theory, it is suggested that ELDs such as GALA induce endosomal leakage by forming pores and flip-flop of membrane lipids following conformational change due to a decrease in pH (Kakudo et al., 2004; Li et al., 2004). In contrast, it is suggested that ELDs such as INF-7 induce endosomal leakage by accumulating in and destabilizing the endosomal membrane (El-Sayed et al., 2009). Accordingly, in the course of endosome maturation, the concomitant decline in pH causes a change in the conformation of the peptide and this destabilizes the endosome membrane leading to the liberation of the endosome contents. The same principle is thought to apply to the toxin A of Pseudomonas (Varkouhi et al., 2011). Following a decline in pH, the conformation of the domain of translocation of the toxin changes, allowing its insertion into the endosome membrane where it forms pores (London, 1992; O'Keefe, 1992). This eventually favors endosome destabilization and translocation of the complex outside of the endosome. The above described ELDs are encompassed within the ELDs of the present description, as well as other mechanisms of endosome leakage whose mechanisms of action may be less well defined.

In some embodiments, the ELD may be an antimicrobial peptide (AMP) such as a linear cationic alpha-helical antimicrobial peptide (AMP). These peptides play a key role in the innate immune response due to their ability to strongly interact with bacterial membranes. Without being bound by theory, these peptides are thought to assume a disordered state in aqueous solution, but adopt an alpha-helical secondary structure in hydrophobic environments. The latter conformation thought to contribute to their typical concentration-dependent membrane-disrupting properties. When accumulated in endosomes at certain concentrations, some antimicrobial peptides may induce endosomal leakage.

In some embodiments, the ELD may be an antimicrobial peptide (AMP) such as Cecropin-A/Melittin hybrid (CM) peptide. Such peptides are thought to be among the smallest and most effective AMP-derived peptides with membrane-disrupting ability. Cecropins are a family of antimicrobial peptides with membrane-perturbing abilities against both Gram-positive and Gram-negative bacteria. Cecropin A (CA), the first identified antibacterial peptide, is composed of 37 amino acids with a linear structure. Melittin (M), a peptide of 26 amino acids, is a cell membrane lytic factor found in bee venom. Cecropin-melittin hybrid peptides have been shown to produce short efficient antibiotic peptides without cytotoxicity for eukaryotic cells (i.e., non-hemolytic), a desirable property in any antibacterial agent. These chimeric peptides were constructed from various combinations of the hydrophilic N-terminal domain of Cecropin A with the hydrophobic N-terminal domain of Melittin, and have been tested on bacterial model systems. Two 26-mers, CA(1-13)M(1-13) and CA(1-8) M(1-18) (Boman et al., 1989), have been shown to demonstrate a wider spectrum and improved potency of natural Cecropin A without the cytotoxic effects of melittin.

In an effort to produce shorter CM series peptides, the authors of Andreu et al., 1992 constructed hybrid peptides such as the 26-mer (CA(1-8)M(1-18)), and compared them with a 20-mer (CA(1-8)M(1-12)), a 18-mer (CA(1-8)M(1-10)) and six 15-mers ((CA(1-7)M(1-8), CA(1-7)M(2-9), CA(1-7)M(3-10), CA(1-7)M(4-11), CA(1-7)M(5-12), and CA(1-7)M(6-13)). The 20 and 18-mers maintained similar activity comparatively to CA(1-8)M(1-18). Among the six 15-mers, CA(1-7)M(1-8) showed low antibacterial activity, but the other five showed similar antibiotic potency compared to the 26-mer without hemolytic effect. Accordingly, in some embodiments, synthetic peptide or polypeptide-based shuttle agents of the present description may comprise an ELD which is or is from CM series peptide variants, such as those described above.

In some embodiments, the ELD may be the CM series peptide CM18 composed of residues 1-7 of Cecropin-A (KWKLFKKIGAVLKVLTTG) fused to residues 2-12 of Melittin (YGRKKRRQRRR), [C(1-7)M(2-12)]. When fused to the cell penetrating peptide TAT, CM18 was shown to independently cross the plasma membrane and destabilize the endosomal membrane, allowing some endosomally-trapped cargoes to be released to the cytosol (Salomone et al., 2012). However, the use of a CM18-TAT11 peptide fused to a fluorophore (atto-633) in some of the authors' experiments, raises uncertainty as to the contribution of the peptide versus the fluorophore, as the use of fluorophores themselves have been shown to contribute to endosomolysis—e.g., via photochemical disruption of the endosomal membrane (Erazo-Oliveras et al., 2014).

In some embodiments, the ELD may be CM18 having the amino acid sequence of SEQ ID NO: 1 of WO/2016/161516, or a variant thereof having at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identity to SEQ ID NO: 1 of WO/2016/161516 and having endosomolytic activity.

In some embodiments, the ELD may be a peptide derived from the N terminus of the HA2 subunit of influenza hemagglutinin (HA), which may also cause endosomal membrane destabilization when accumulated in the endosome.

In some embodiments, synthetic peptide or polypeptide-based shuttle agents of the present description may comprise an ELD which is or is from an ELD set forth in Table I, or a variant thereof having endosome escape activity and/or pH-dependent membrane disrupting activity.

TABLE I
Examples of endosome leakage domains

| | SEQ ID NO of | |
|---|---|---|
| Name | WO/2016/161516 | Reference(s) |
| CM18 | 1 | Salomone et al., 2012 |
| Diphtheria toxin | 2 | Uherek et al., 1998; |
| T domain (DT) | | Glover et al., 2009 |
| GALA | 3 | Parente et al., 1990; |
| | | Li et al., 2004 |
| PEA | 4 | Fominaya and Wels 1996 |
| INF-7 | 5 | El-Sayed et al., 2009 |
| LAH4 | 6 | Kichler et al., 2006; |
| | | Kichler et al., 2003 |
| HGP | 7 | Kwon et al., 2010 |
| H5WYG | 8 | Midoux et al., 1998 |
| HA2 | 9 | Lorieau et al., 2010 |
| EB1 | 10 | Amand et al., 2012 |
| VSVG | 11 | Schuster et al., 1999 |
| Pseudomonas toxin | 12 | Fominaya et al., 1998 |
| Melittin | 13 | Tan et al., 2012 |
| KALA | 14 | Wyman et al., 1997 |
| JST-1 | 15 | Gottschalk et al., 1996 |
| C(LLKK)₃C | 63 | Luan et al., 2015 |
| G(LLKK)₃G | 64 | Luan et al., 2015 |

In some embodiments, shuttle agents of the present description may comprise one or more ELD or type of ELD. More particularly, they can comprise at least 2, at least 3, at least 4, at least 5, or more ELDs. In some embodiments, the shuttle agents can comprise between 1 and 10 ELDs, between 1 and 9 ELDs, between 1 and 8 ELDs, between 1 and 7 ELDs, between 1 and 6 ELDs, between 1 and 5 ELDs, between 1 and 4 ELDs, between 1 and 3 ELDs, etc.

In some embodiments, the order or placement of the ELD relative to the other domains (CPD, histidine-rich domains) within the shuttle agents of the present description may be varied provided the shuttling ability of the shuttle agent is retained.

In some embodiments, the ELD may be a variant or fragment of any one those listed in Table I, and having endosomolytic activity. In some embodiments, the ELD may comprise or consist of the amino acid sequence of any one of SEQ ID NOs: 1-15, 63, or 64 of WO/2016/161516, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 1-15, 63, or 64 of WO/2016/161516, and having endosomolytic activity.

In some embodiments, shuttle agents of the present description do not comprise one or more of the amino acid sequences of any one of SEQ ID NOs: 1-15, 63, or 64 of WO/2016/161516.

### Cell Penetration Domains (CPDs)

In some aspects, the shuttle agents of the present description may comprise a cell penetration domain (CPD). As used herein, the expression “cell penetration domain” refers to a sequence of amino acids which confers the ability of a macromolecule (e.g., peptide or protein) containing the CPD to be transduced into a cell.

In some embodiments, the CPD may be (or may be from) a cell-penetrating peptide or the protein transduction domain of a cell-penetrating peptide. Cell-penetrating peptides can serve as carriers to successfully deliver a variety of cargoes intracellularly (e.g., polynucleotides, polypeptides, small molecule compounds or other macromolecules/compounds that are otherwise membrane-impermeable). Cell-penetrating peptides often include short peptides rich in basic amino acids that, once fused (or otherwise operably linked) to a macromolecule, mediate its internalization inside cells (Shaw et al., 2008). The first cell-penetrating peptide was identified by analyzing the cell penetration ability of the HIV-1 trans-activator of transcription (Tat) protein (Green and Loewenstein 1988, Vives et al., 1997). This protein contains a short hydrophilic amino acid sequence, named “TAT”, which promotes its insertion within the plasma membrane and the formation of pores. Since this discovery, many other cell-penetrating peptides have been described. In this regard, in some embodiments, the CPD can be a cell-penetrating peptide as listed in Table II, or a variant thereof having cell-penetrating activity.

TABLE II
Examples of cell-penetrating peptides

| | SEQ ID NO of | |
|---|---|---|
| Name | WO/2016/161516 | Reference(s) |
| SP | 16 | Mahlum et al., 2007 |
| TAT | 17 | Green and Loewenstein 1988; |
| | | Fawell et al., 1994; |
| | | Vives et al., 1997 |
| Penetratin | 18 | Perez et al., 1992 |
| (Antennapedia) | | |
| pVEC | 19 | Elmquist et al., 2001 |
| M918 | 20 | El-Andaloussi et al., 2007 |
| Pep-1 | 21 | Morris et al., 2001 |
| Pep-2 | 22 | Morris et al., 2004 |
| Xentry | 23 | Montrose et al., 2013 |
| Arginine stretch | 24 | Zhou et al., 2009 |
| Transportan | 25 | Hallbrink et al., 2001 |
| SynB1 | 26 | Drin et al., 2003 |
| SynB3 | 27 | Drin et al., 2003 |
| PTD4 | 65 | Ho et al, 2001 |

Without being bound by theory, cell-penetrating peptides are thought to interact with the cell plasma membrane before crossing by pinocytosis or endocytosis. In the case of the TAT peptide, its hydrophilic nature and charge are thought to promote its insertion within the plasma membrane and the formation of a pore (Herce and Garcia, 2007). Alpha helix motifs within hydrophobic peptides (such as SP) are also thought to form pores within plasma membranes (Veach et al., 2004).

In some embodiments, shuttle agents of the present description may comprise one or more CPD or type of CPD. More particularly, they may comprise at least 2, at least 3, at least 4, or at least 5 or more CPDs. In some embodiments, the shuttle agents can comprise between 1 and 10 CPDs, between 1 and 6 CPDs, between 1 and 5 CPDs, between 1 and 4 CPDs, between 1 and 3 CPDs, etc.

In some embodiments, the CPD may be TAT having the amino acid sequence of SEQ ID NO: 17 of WO/2016/161516, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 90%, 91%, 92%, 93%, 94%, or 95% identity to SEQ ID NO: 17 of WO/2016/161516 and having cell penetrating activity; or Penetratin having the amino acid sequence of SEQ ID NO: 18 of WO/2016/161516, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to SEQ ID NO: 18 of WO/2016/161516 and having cell penetrating activity.

In some embodiments, the CPD may be PTD4 having the amino acid sequence of SEQ ID NO: 65 of WO/2016/161516, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 90%, 91%, 92%, 93%, 94%, or 95% identity to SEQ ID NO: 65 of WO/2016/161516.

In some embodiments, the order or placement of the CPD relative to the other domains (ELD, histidine-rich domains) within the shuttle agents of the present description may be varied provided the transduction ability of the shuttle agent is retained.

In some embodiments, the CPD may be a variant or fragment of any one those listed in Table II, and having cell penetrating activity. In some embodiments, the CPD may comprise or consist of the amino acid sequence of any one of SEQ ID NOs: 16-27 or 65 of WO/2016/161516, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 16-27 or 65 of WO/2016/161516, and having cell penetrating activity.

In some embodiments, shuttle agents of the present description do not comprise any one of the amino acid sequences of SEQ ID NOs: 16-27 or 65 of WO/2016/161516.

### Methods, Kits, Uses, Compositions, and Cells

In some embodiments, the present description relates to methods for delivering cargoes from an extracellular space to the cytosol and/or nucleus of a target eukaryotic cell. The methods comprise contacting the target eukaryotic cell with the cargo in the presence of a shuttle agent at a concentration sufficient to increase the transduction efficiency of said cargo, as compared to in the absence of said shuttle agent. In some embodiments, contacting the target eukaryotic cell with the cargo in the presence of the shuttle agent results in an increase in the transduction efficiency of said cargo by at least 3, 3.5, 4, 4.5, 5, 5.5, 6, 6.5, 7, 7.5, 8, 8.5, 9, 9.5, 10, 20, 30, 40, 50, or 100-fold, as compared to in the absence of said shuttle agent. In some embodiments, the concentration of cargo and/or of synthetic peptide shuttle agent in compositions described herein may be at least 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μM.

In some embodiments, the present description relates to a method for increasing the transduction efficiency of a cargo to the cytosol and/or nucleus of target eukaryotic cells. As used herein, the expression “increasing transduction efficiency” refers to the ability of a shuttle agent of the present description to improve the percentage or proportion of a population of target cells into which a cargo of interest is delivered intracellularly. Immunofluorescence microscopy, flow cytometry, and other suitable methods may be used to assess cargo transduction efficiency. In some embodiments, a shuttle agent of the present description may enable a transduction efficiency of at least 25%, 30%, 35%, 40%, 45%, 50%, 55%, 60%, 65%, 70%, 75%, 80%, or 85%, for example as measured by immunofluorescence microscopy, flow cytometry, FACS, and other suitable methods. In some embodiments, a shuttle agent of the present description may enable one of the aforementioned transduction efficiencies together wish a cell viability of at least 25%, 30%, 35%, 40%, 45%, 50%, 55%, 60%, 65%, 70%, 75%, 80%, 85%, 90%, or 95%, for example as measured by the assay described in Example 3.3a of WO/2018/068135, or by another suitable assay known in the art.

In addition to increasing target cell transduction efficiency, shuttle agents of the present description may facilitate the delivery of a cargo of interest to the cytosol and/or nucleus of target cells. In this regard, efficiently delivering an extracellular cargo to the cytosol and/or nucleus of a target cell using peptides can be challenging, as the cargo often becomes trapped in intracellular endosomes after crossing the plasma membrane, which may limit its intracellular availability and may result in its eventual metabolic degradation. For example, use of the protein transduction domain from the HIV-1 Tat protein has been reported to result in massive sequestration of the cargo into intracellular vesicles. In some aspects, shuttle agents of the present description may facilitate the ability of endosomally-trapped cargo to escape from the endosome and gain access to the cytoplasmic compartment. In this regard, the expression “to the cytosol” for example in the phrase “increasing the transduction efficiency of a cargo to the cytosol,” is intended to refer to the ability of shuttle agents of the present description to allow an intracellularly delivered cargo of interest to escape endosomal entrapment and gain access to the cytoplasmic and/or nuclear compartment. After a cargo of interest has gained access to the cytosol, it may be free to bind to its intracellular target (e.g., in the cytosol, nucleus, nucleolus, mitochondria, peroxisome). In some embodiments, the expression “to the cytosol” is thus intended to encompass not only cytosolic delivery, but also delivery to other subcellular compartments that first require the cargo to gain access to the cytoplasmic compartment.

In some embodiments, the methods of the present description are in vitro methods (e.g., such as for therapeutic and/or diagnostic purpose). In other embodiments, the methods of the present description are in vivo methods (e.g., such as for therapeutic and/or diagnostic purpose). In some embodiments, the methods of the present description comprise topical, enteral/gastrointestinal (e.g., oral), or parenteral administration of the cargo and the synthetic peptide shuttle agent. In some embodiments, described herein are compositions formulated for topical, enteral/gastrointestinal (e.g., oral), or parenteral administration of the cargo and the synthetic peptide shuttle agent.

In some embodiments, the methods of the present description may comprise contacting the target eukaryotic cell with the shuttle agent, or composition as defined herein, and the cargo. In some embodiments, the shuttle agent, or composition may be pre-incubated with the cargo to form a mixture, prior to exposing the target eukaryotic cell to that mixture. In some embodiments, the type of shuttle agent may be selected based on the identity and/or physicochemical properties of the cargo to be delivered intracellularly. In other embodiments, the type of shuttle agent may be selected to take into account the identity and/or physicochemical properties of the cargo to be delivered intracellularly, the type of cell, the type of tissue, etc.

In some embodiments, the method may comprise multiple treatments of the target cells with the shuttle agent, or composition (e.g., 1, 2, 3, 4 or more times per day, and/or on a pre-determined schedule). In such cases, lower concentrations of the shuttle agent, or composition may be advisable (e.g., for reduced toxicity). In some embodiments, the cells may be suspension cells or adherent cells. In some embodiments, the person of skill in the art will be able to adapt the teachings of the present description using different combinations of shuttles, domains, uses and methods to suit particular needs of delivering a cargo to particular cells with a desired viability.

In some embodiments, the methods of the present description may apply to methods of delivering a cargo intracellularly to a cell in vivo. Such methods may be accomplished by parenteral administration or direct injection into a tissue, organ, or system.

In some aspects, the compositions or synthetic peptide shuttle agents of the present description may be for use in an in vitro or in vivo method for increasing the transduction efficiency of a cargo (e.g., a therapeutically or biologically relevant molecule or drug) into target eukaryotic cells, wherein the synthetic peptide shuttle agent or synthetic peptide shuttle agent variant is used or is formulated for use at a concentration sufficient to increase the transduction efficiency and cytosolic and/or nuclear delivery of the cargo into the target eukaryotic cells, as compared to in the absence of the synthetic peptide shuttle agent or synthetic peptide shuttle agent variant.

In some embodiments, compositions or synthetic peptide shuttle agents of the present description may be for use in therapy, wherein the synthetic peptide shuttle agent or synthetic peptide shuttle agent variant transduces a therapeutically relevant cargo to the cytosol and/or nucleus of target eukaryotic cells, wherein the synthetic peptide shuttle agent or synthetic peptide shuttle agent variant is used (or is formulated for use) at a concentration sufficient to increase the transduction efficiency of the cargo into the target eukaryotic cells, as compared to in the absence of the synthetic peptide shuttle agent.

In some aspects, described herein is a composition for use in transducing a cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic and/or nuclear delivery of the cargo into said target eukaryotic cells upon administration, as compared to in the absence of said synthetic peptide shuttle agent. In some embodiments, the composition further comprises the cargo. In some embodiments, the composition may be mixed with the cargo prior to administration or therapeutic use.

In some aspects, described herein is a composition for use in therapy, the composition comprising a synthetic peptide shuttle agent formulated with a cargo to be transduced into target eukaryotic cells by the synthetic peptide shuttle agent, wherein the concentration of the synthetic peptide shuttle agent in the composition is sufficient to increase the transduction efficiency and cytosolic and/or nuclear delivery of the cargo into said target eukaryotic cells upon administration, as compared to in the absence of said synthetic peptide shuttle agent.

In some aspects, described herein is a composition: (a) for use in increasing the transduction efficiency of the nucleoprotein cargo to the cytosolic/nuclear compartment of eukaryotic cells; (b) for use in genome editing, base editing, or prime editing in eukaryotic cells; (c) for use in modulating gene expression in the eukaryotic cells; (d) for use in therapy, wherein the nucleoprotein cargo binds to a therapeutic target in the eukaryotic cells; (e) for use in delivering a non-therapeutic nucleoprotein cargo as a diagnostic agent; (f) for use in the manufacture of a medicament or diagnostic agent; (g) for use in treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis); or (h) any combination of (a) to (g).

In some aspects, described herein is a composition comprising a cargo for intracellular delivery and a synthetic peptide shuttle agent that is independent from, or is not covalently linked to, said cargo, the synthetic peptide shuttle agent being a peptide comprising an amphipathic alpha-helical motif having both a positively-charged hydrophilic outer face and a hydrophobic outer face, wherein synthetic peptide shuttle agent increases cytosolic/nuclear delivery of said cargo in eukaryotic cells as compared to in the absence of the synthetic peptide shuttle agent. In some embodiments, the compositions and/or shuttle agents described herein do not comprise an organic solvent (e.g., DMSO), or do not comprise a concentration of an organic solvent not suitable for therapeutic or human use. In some embodiments, the shuttle agents described herein are advantageously designed with aqueous solubility in mind, thereby precluding the necessity of using organic solvents.

In some embodiments, the shuttle agent, or composition, and the cargo may be exposed to the target cell in the presence or absence of serum. In some embodiments, the method may be suitable for clinical or therapeutic use.

In some embodiments, the present description relates to a kit for delivering a cargo from an extracellular space to the cytosol and/or nucleus of a target eukaryotic cell. In some embodiments, the present description relates to a kit for increasing the transduction efficiency of a cargo to the cytosol of a target eukaryotic cell. The kit may comprise the shuttle agent, or composition as defined herein, and a suitable container.

In some embodiments, the target eukaryotic cells may be an animal cell, a mammalian cell, or a human cell. In some embodiments, the target eukaryotic cells may be stem cells (e.g., embryonic stem cells, pluripotent stem cells, induced pluripotent stem cells, neural stem cells, mesenchymal stem cells, hematopoietic stem cells, peripheral blood stem cells), primary cells (e.g., myoblast, fibroblast), immune cells (e.g., NK cell, T cell, dendritic cell, antigen presenting cell), epithelial cells, skin cells, gastrointestinal cells, mucosal cells, or pulmonary (lung) cells. In some embodiments, target cells comprise those having the cellular machinery for endocytosis (i.e., to produce endosomes).

In some embodiments, the present description relates to an isolated cell comprising a synthetic peptide shuttle agent as defined herein. In some embodiments, the cell may be a pluripotent stem cell. It will be understood that cells that are often resistant or not amenable to DNA transfection may be interesting candidates for the synthetic peptide shuttle agents of the present description.

Synthetic peptide shuttle agents have been shown to enable efficient delivery of recombinant protein cargoes to refractory airway epithelial cells (Krishnamurthy et al., 2018). Mucus/sputum, particularly in subjects with respiratory diseases (e.g., cystic fibrosis), is known to be elevated in DNA (Chance et al., 2020), which may have an inhibitory effect on some synthetic peptide shuttle agents. In some aspects, described herein is a synthetic peptide shuttle agent for use in, or suitable for use in, the delivery of non-anionic cargoes across mucus-producing membranes (e.g., airway epithelium), the synthetic peptide shuttle agent comprising or consisting essentially of a central core amphipathic alpha helical region having shuttle agent activity, flanked N- and C-terminally by flexible linker domains, wherein one or both of the flexible linker domains comprises or consists essentially of a sufficient number of non-cationic hydrophilic residues such that cargo transduction activity across mucus-producing membranes of the synthetic peptide shuttle agent is increased relative to that of the central core amphipathic alpha helical region lacking the flexible linker domains. In some embodiments, the central core amphipathic alpha helical region: (a) may be an endosomolytic peptide; (b) may be at least 8, 9, 10, 11, 12, 13, 14, 15, 16, 17, 18, or 19 amino acids in length; (c) may be a fragment of a parent shuttle agent as defined in claim 14(a) or 15; (d) may be an amphipathic helix as defined in any one of claims 18 to 29 or 49 to 60; (e) may have a hydrophobic moment (μH) of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5; or (f) any combination of (a) to (e). In some embodiments, the non-cationic hydrophilic residues may comprise or consist essentially of glycine, serine, aspartate, glutamate, histidine, tyrosine, threonine, cysteine, asparagine, glutamine, or any combination thereof. In some embodiments, the flexible linker domain is any linker domain as defined herein.

### EXAMPLES

### Example 1: Materials and Methods

All materials and methods not described or specified herein were generally as performed in WO/2018/068135, CA 3,040,645, WO/2020/210916, or PCT/CA2021/051458. Materials and reagents used in the Examples herein are shown in Table III. All cell lines were grown according to the manufacture's instructions, as shown in Table IV.

### Helical Wheel Projections and Generation of 3D Peptide Images

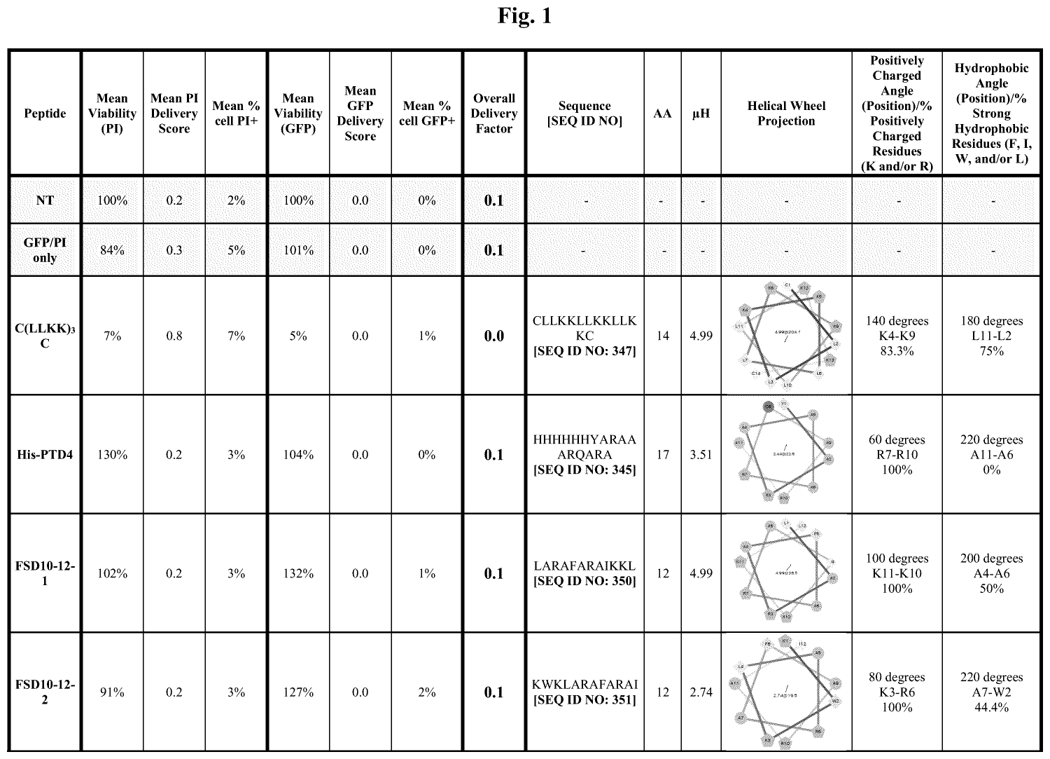

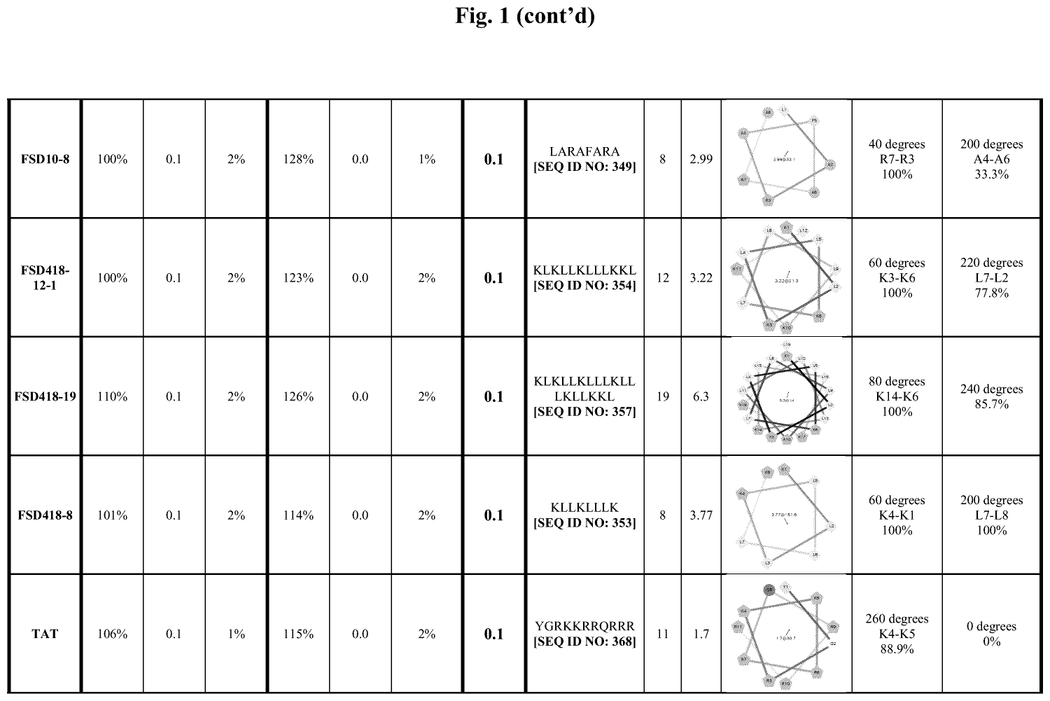

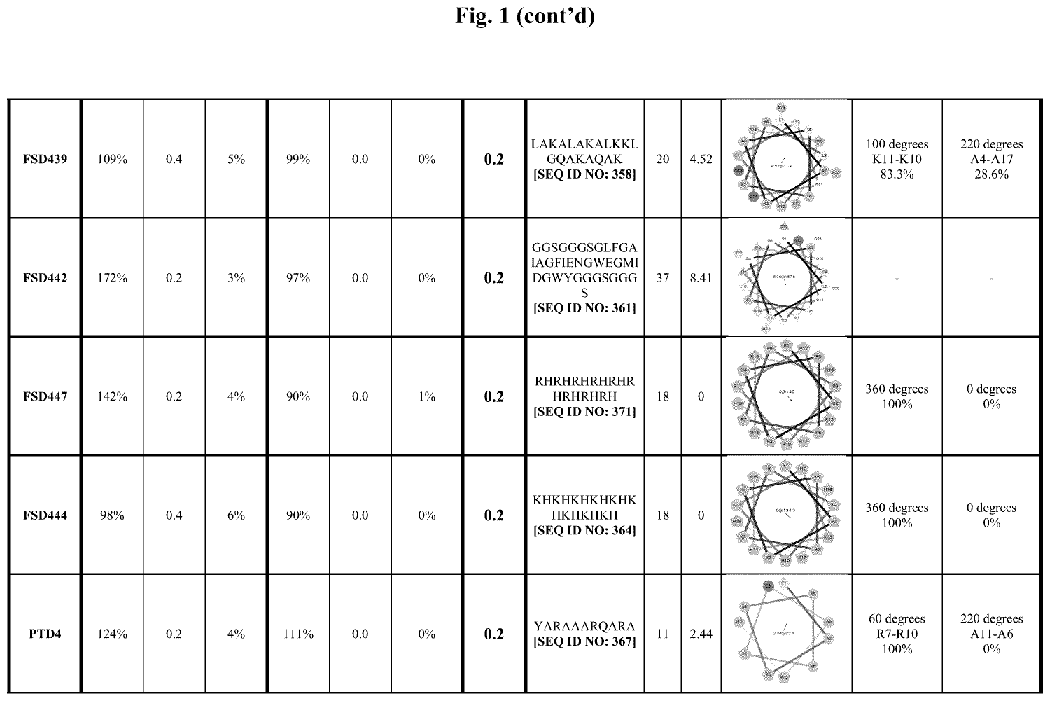

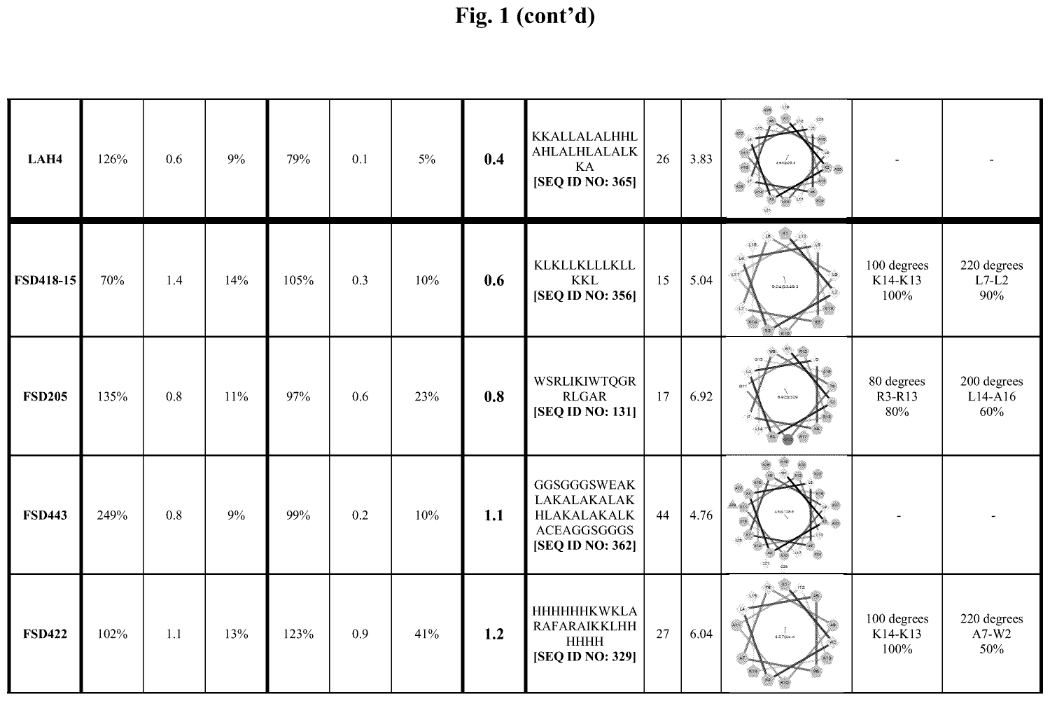

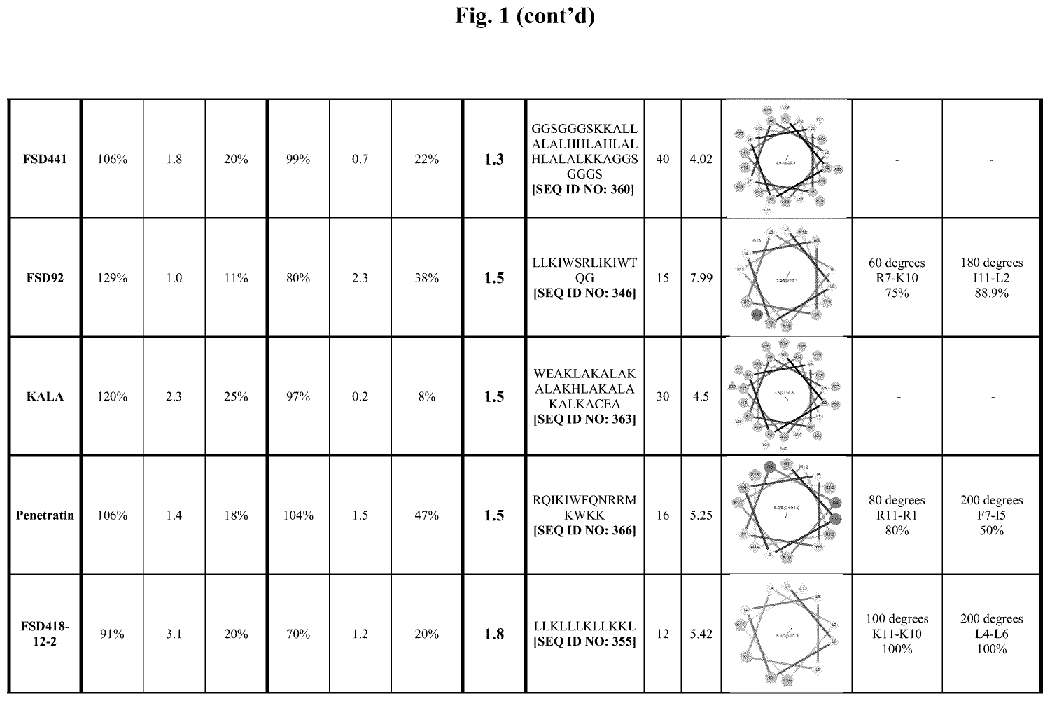

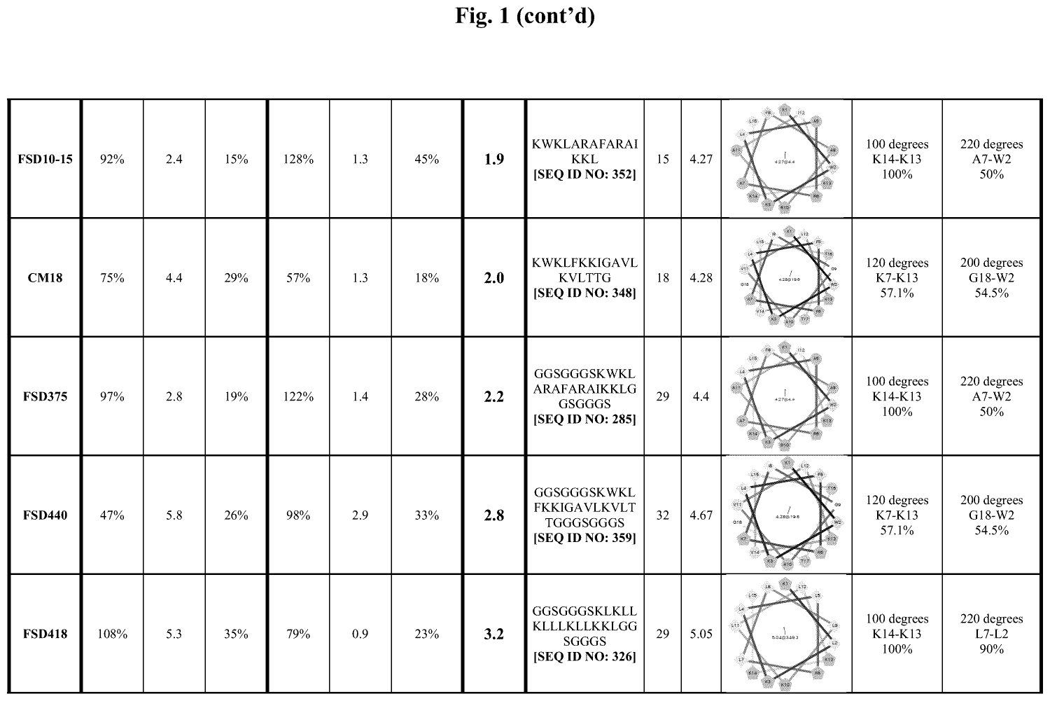

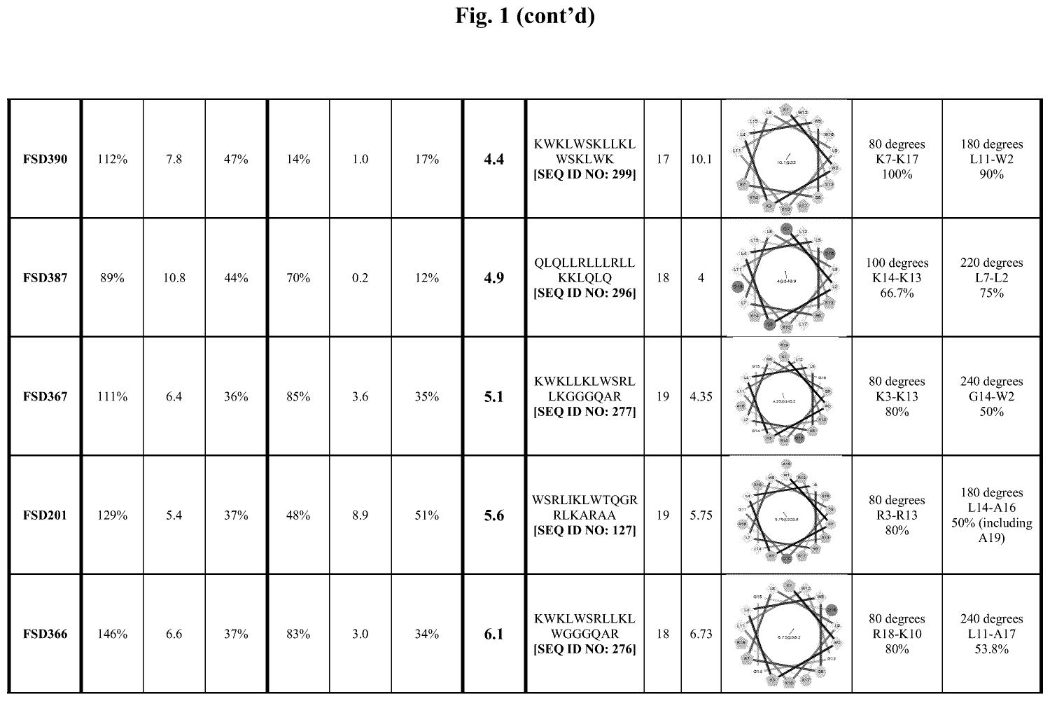

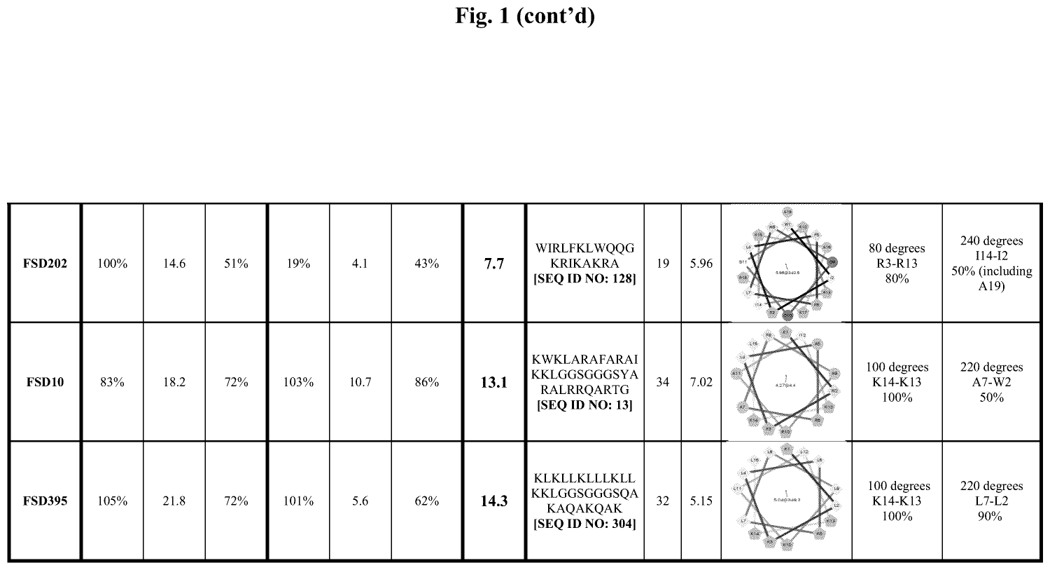

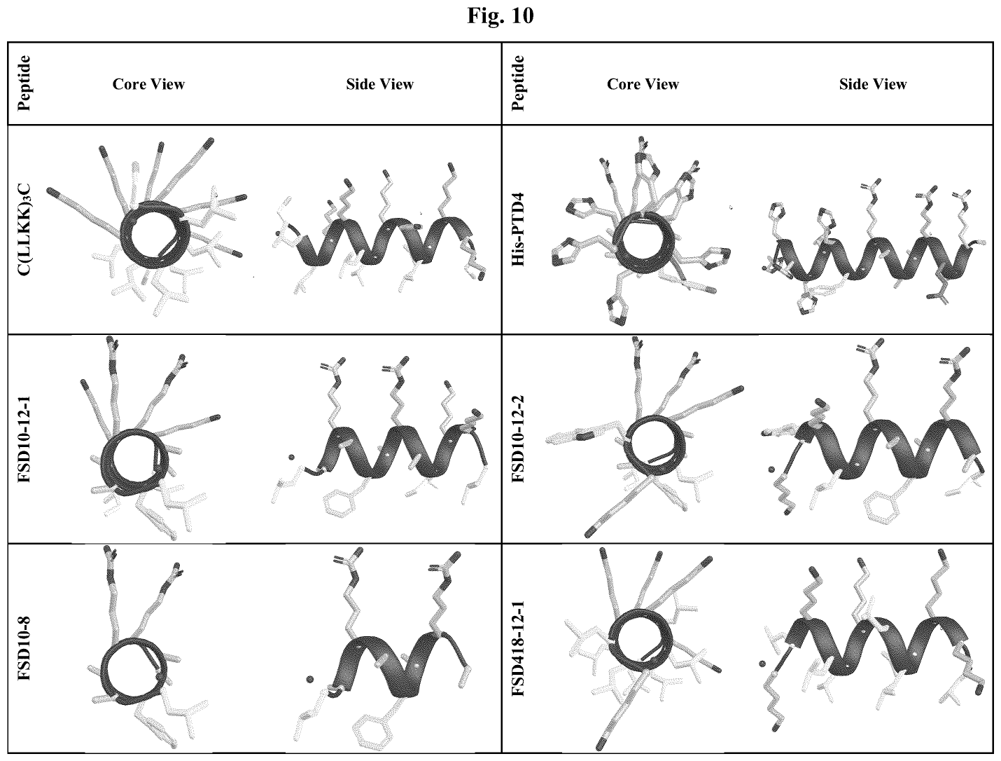

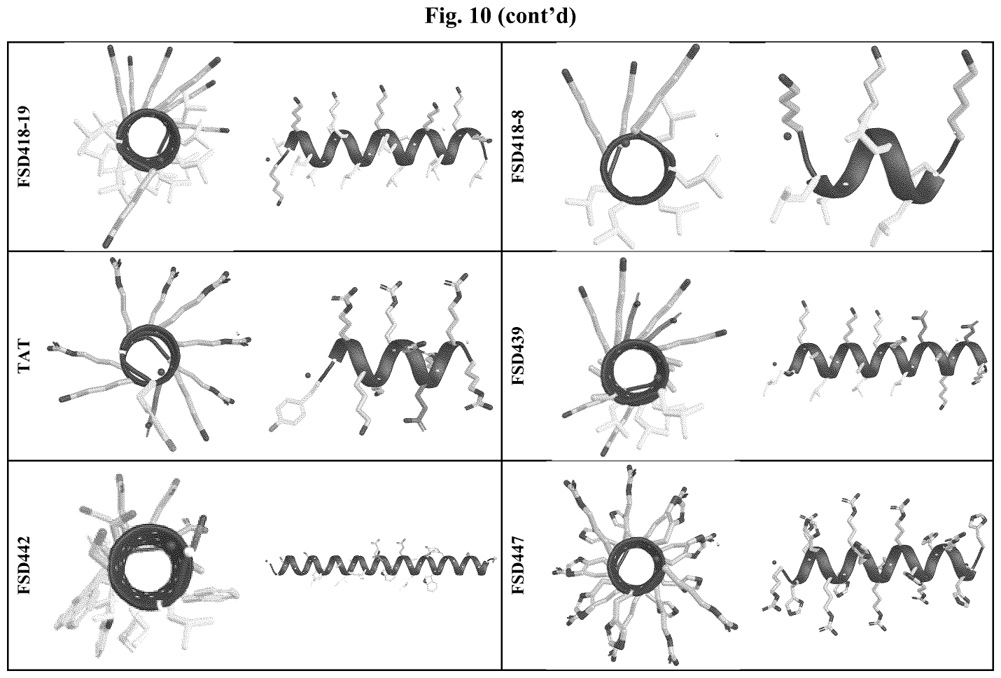

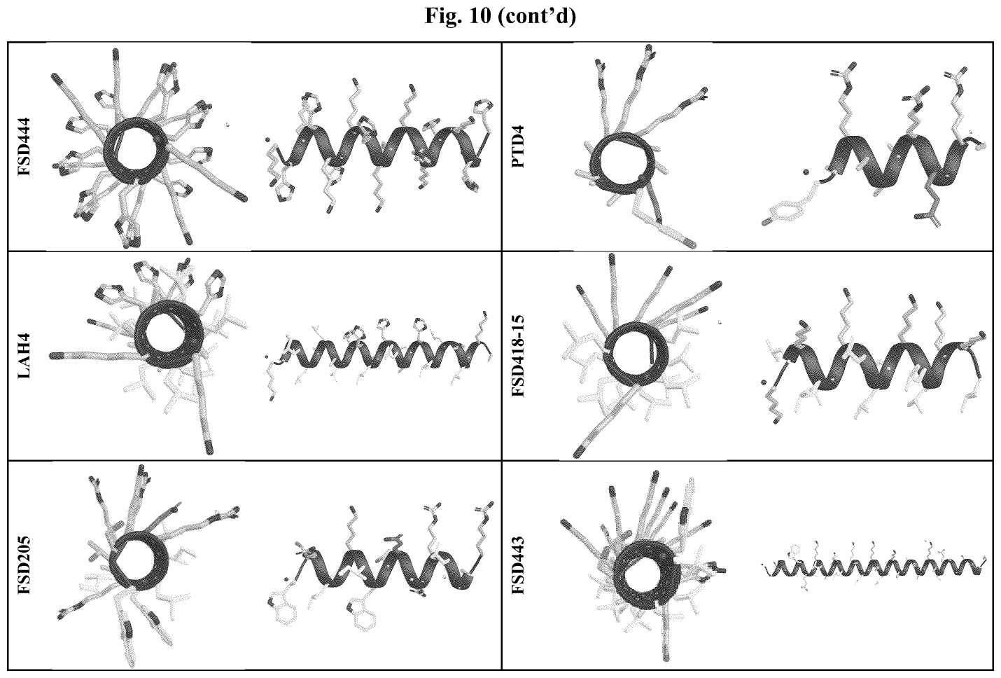

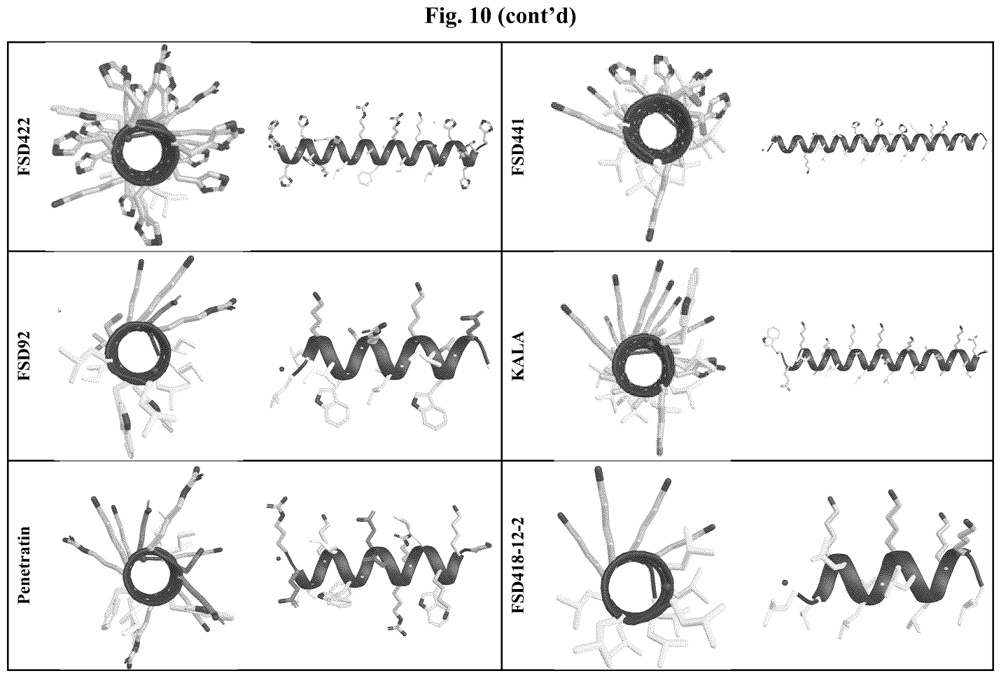

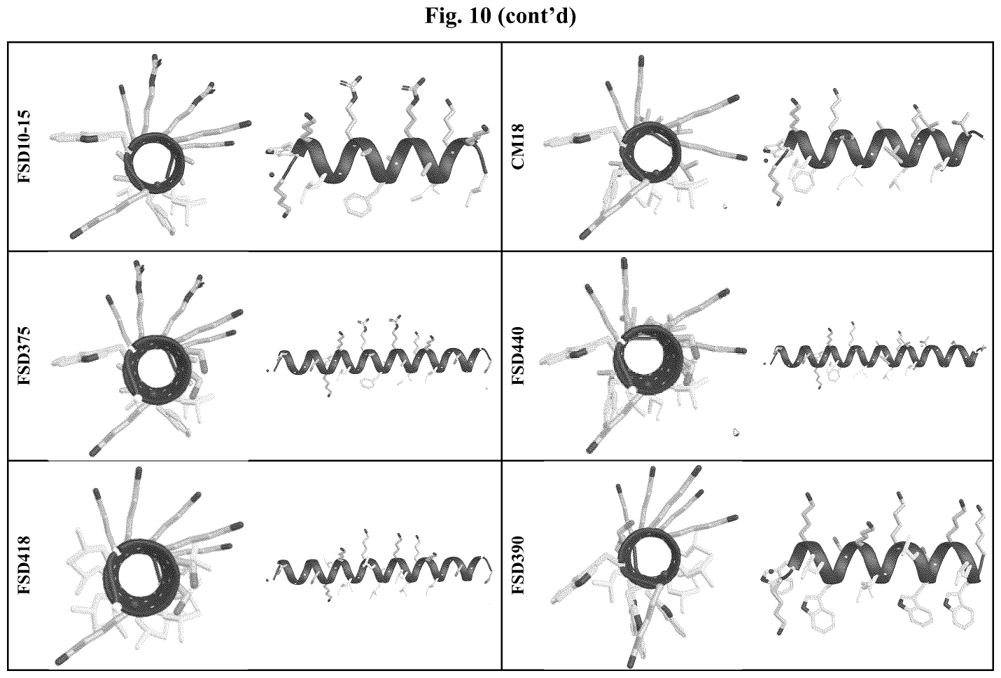

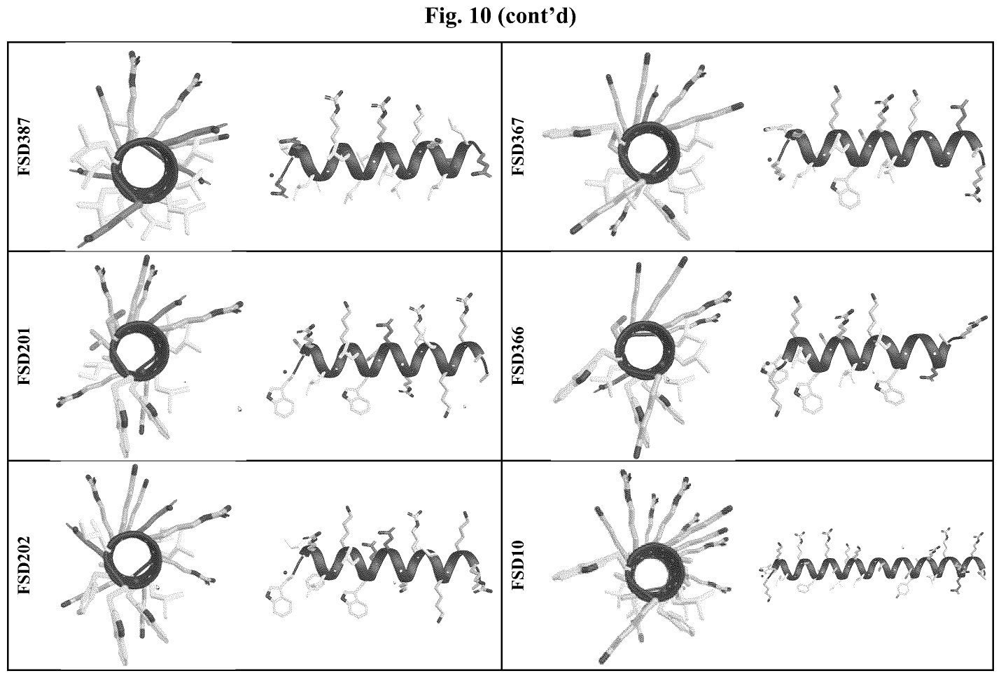

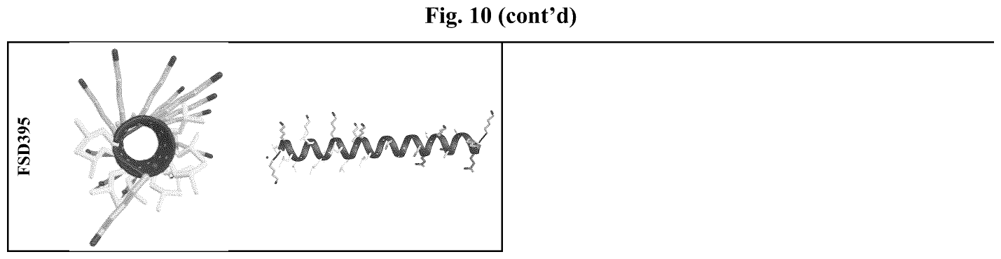

Helical wheel projection images of the synthetic peptide shuttle agents in FIG. 1 were generated using an online helical wheel projection tool created by Don Armstrong and Raphael Zidovetzki. (e.g., available at: https://www.donarmstrong.com/cgi-bin/wheel.pl). In FIG. 10, 3D peptide structures were built in PyMol (open-source version for Linux Version 2.4.0a0) using the “fab” command and the “ss=1” argument for the peptide to adopt an alpha helix conformation. Orientation was done manually. Colouring was done using a script, whereby varying shades of green represent strongly hydrophobic residues (Y, W, I, M, L, F), with darker green representing highly hydrophobic residues; blue residues represent charged hydrophilic residues (K, H, R, E, D); red residues represent uncharged hydrophilic residues (Q, N); and yellow/orange residues represent weakly hydrophobic residues (G, A, S, T).

### Transduction Protocol

Transduction of GFP-NLS

HeLa cells were plated (20 000 cells/well) in a 96 well-plate the day prior to the experiment in DMEM containing 10% FBS. Each delivery mix comprising a synthetic peptide shuttle agent (10-30 μM) and GFP-NLS (10 μM) was prepared and completed to 50 μL with RPMI 1640 media. Cells were washed once with phosphate-buffered saline (PBS) and the shuttle/GFP-NLS mix was added to the cells and incubated for five minutes. Then 100 μL DMEM containing 10% FBS was added to the mix. Cells were then immediately washed once with PBS and incubated in DMEM containing 10% FBS for two hours. Cells were then analyzed by flow cytometry.

Transduction of PI

HeLa cells were plated (20 000 cells/well) in a 96 well-plate the day prior to the experiment in DMEM containing 10% FBS. Each delivery mix comprising a synthetic peptide shuttle agent (10-30 μM) and the propidium iodide (PI) (10 μg/mL) were prepared and completed to 50 μL with PBS. Cells were washed once with PBS and the shuttle/PI mix was added to the cells and incubated for one minute. Then 100 μL DMEM containing 10% FBS was added to the mix. Cells were then immediately washed once with PBS and incubated in DMEM containing 10% FBS for two hours. Cells were then analyzed by flow cytometry.

TABLE III
Materials and Reagents

| | | City, |
|---|---|---|
| Materials | Company | Province-State, Country |
| DMEM | Sigma-Aldrich | Oakville, ON, Canada |
| Fetal bovine serum (FBS) | NorthBio | Toronto, ON, Canada |
| L-glutamine-Penicillin- | Sigma-Aldrich | Oakville, ON, Canada |
| Streptomycin | | |
| RPMI 1640 media | Sigma-Aldrich | Oakville, ON, Canada |
| Trypsin-EDTA solution | Sigma-Aldrich | Oakville, ON, Canada |
| Propidium iodide | Sigma Aldrich/P4170-10MG | Oakville, ON, Canada |
| Alpha-MEM | Sigma-Aldrich | Oakville, ON, Canada |
| Edit-R ™ TracrRNA | Horizon Discovery/ | Lafayette, CO, USA |
| | Dharmacon (U-002005-50) | |
| crRNA Beta-2 microglobulin (B2M) | IDT | Mississauga, ON, Canada |
| for Cas9 | | |
| GAGUAGCGCGAGCACAGCUA | | |
| [SEQ ID NO: 372] | | |
| crRNA Beta-2 microglobulin (B2M) | IDT | Mississauga, ON, Canada |
| for Cas12a | | |
| AGUGGGGGUGAAUUCAGUGUAGU | | |
| [SEQ ID NO: 373] | | |
| 1,3-Diaminoguanidine | Sigma-Aldrich (#143413) | Oakville, ON, Canada |
| monohydrochloride | | |
| 3,5-Diamino-1,2,4-triazole | Sigma-Aldrich (#D26202) | Oakville, ON, Canada |
| Guanidine hydrochloride | Sigma-Aldrich (#G3272) | Oakville, ON, Canada |
| L-Arginine amide dihydrochloride | Santa Cruz biotech. | Dallas, TX, USA |
| | (sc-286061) | |
| Anti-beta 2 Microglobulin antibody- | Abcam (#ab49424) | Toronto, ON, Canada |
| PE conjugated | | |
| Bovine Serum Albumin (BSA) | BioShop Canada Inc. | Burlington, ON, Canada |
| | #ALB007.100 | |

TABLE IV
Cell lines

| Cell | | | Culture | | |
|---|---|---|---|---|---|
| lines | Description | ATCC/others | media | Serum | Additives |
| HeLa | Human cervical | ATCC ™ | DMEM | 10% FBS | L-glutamine 2 mM |
| | carcinoma cells | CCL-2 | | | Penicillin 100 units |
| | | | | | Streptomycin 100 μg/mL |
| CFF- | Immortalized | Provided by the | Alpha-MEM | 10% FBS | L-glutamine 2 mM |
| 16HBEge | Human bronchial | Cystic Fibrosis | | | Penicillin 100 units |
| | epithelial cells | Foundation | | | Streptomycin 100 μg/mL |
| RH-30 | Human | ATCC ™ | RPMI-1640 | 10% FBS | L-glutamine 2 mM |
| | rhabdomyosarcoma | CRL-2061 | | | Penicillin 100 units |
| | cell line | | | | Streptomycin 100 μg/mL |

### GFP-NLS Transduction in the Presence of Cas9-RNP Complex

HeLa cells were plated (20 000 cells/well) in a 96 well-plate the day prior to the experiment in DMEM containing 10% FBS. A mix was prepared containing a synthetic peptide shuttle agent (10-30 μM), the GFP-NLS (10 μM) with or without a Cas9-NLS recombinant protein (2.5 μM) complexed with a crRNA/tracrRNA (2 μM) targeting the beta-2 microglobulin (B2M) gene and completed to 50 μL with PBS. Cells were washed once with PBS and the shuttle/GFP-NLS/Cas9-RNP mix was added to the cells and incubated for one minute. Then 100 μL DMEM containing 10% FBS was added to the mix. Cells were then immediately washed once with PBS and incubated in DMEM containing 10% FBS for two hours. Cells were the analyzed by flow cytometry.

GFP-NLS Transduction in Presence of Cas9-RNP Complex Coated with Small Molecules Protocol

HeLa cells were plated (20 000 cells/well) in a 96 well-plate the day prior to the experiment in DMEM containing 10% FBS. The Cas9-RNP complex coated with small molecules was prepared by mixing a Cas9-NLS recombinant protein (5 μM) complexed with a crRNA/tracrRNA (4 μM) targeting the beta-2 microglobulin (B2M) gene with 0, 100 nM, 1 μM, 10 μM, 100 μM, 1 mM or 10 mM of either 1,3-diaminoguanidine monohydrochloride, 3,5-diamino-1,2,4-triazole, guanidine hydrochloride or L-arginine amide dihydrochloride. The Cas9 RNP complex coated with small molecules was completed to 254 with PBS. The delivery mix was prepared by mixing a synthetic peptide shuttle agent (10 μM), the GFP-NLS (10 μM) with or without the Cas9 RNP complex coated or not with small molecules and completed to 50 μL with phosphate-buffered saline PBS. Cells were washed once with PBS and the shuttle/GFP-NLS/Cas9-RNP mix was added to the cells and incubated for one minute. Then 100 μL DMEM containing 10% FBS was added to the mix. Cells were then immediately washed once with PBS and incubated in DMEM containing 10% FBS for two hours. Cells were then analyzed by flow cytometry.

### Transduction of CRISPR Cas9- or Cpf1-RNP

Transduction

HeLa cells were plated (10 000 cells/well) in a 96 well-plate the day prior to the experiment in DMEM containing 10% FBS. CFF-16HBEge cells were plated (10 000 cells/well) in a 96 well-plate the day prior to the experiment in Alpha-MEM containing 10% FBS.

For Cpf1-RNP transduction, a mix of Cpf1-NLS recombinant protein (1.33 μM) complexed with a crRNA (2 μM) targeting the beta-2 microglobulin (B2M) gene were co-incubated with 10-20 μM of synthetic peptide shuttle agent in a final volume of 504 completed with PBS. Cells were washed once with PBS and the shuttle/Cpf1-RNP mix was added to the cells and incubated for 90 seconds. Then 100 μL of DMEM (HeLa) or Alpha-MEM (CFF-16HBEge) containing 10% FBS was added to the mix. Cells were then immediately washed once with PBS and incubated in DMEM (HeLa) or Alpha-MEM (CFF-16HBEge) containing 10% FBS.

For Cas9-RNP or ABE-Cas9-RNP transduction, a mix of Cas9-NLS or ABE-Cas9 recombinant protein (2.5 μM) complexed with a crRNA/tracrRNA (2 μM) targeting the beta-2 microglobulin (B2M) gene were co-incubated with 10-20 μM of synthetic peptide shuttle agent in a final volume of 50 μL completed with PBS. Cells were washed once with PBS and the shuttle/Cas9-RNP complex was added to the cells for and incubated for 60 to 90 seconds. Then 100 μL of DMEM (HeLa) or Alpha-MEM (CFF-16HBEge) containing 10% FBS was added to the mix. Cells were then immediately washed once with PBS and incubated in DMEM (HeLa) or Alpha-MEM (CFF-16HBEge) containing 10% FBS.

Knockout Analysis by Flow Cytometry

Genome editing events resulting in the absence of B2M protein (knockout) at the cell surface were determined by flow cytometry 6 days post-transduction. Cells were washed once with PBS and incubated with the anti-B2 microglobulin antibody (PE conjugated) (0.5 μL of anti-B2M-PE in 504 0.5% BSA/PBS) for 45 minutes at room temperature. Cells were washed twice with PBS and detached with 504 of Trypsin-EDTA for 10 minutes at 37° C. then inactivated by adding 100 μL of media containing 10% FBS. The percentage of knockout cells (cells without B2M antibody signal) was determined by flow cytometry.

For all transduction experiments, cell viabilities were above 75% unless otherwise indicated.

### Example 2: Synthetic Peptide Shuttle Agents: A New Class of Intracellular Delivery Peptides

Synthetic peptides called shuttle agents represent a new class of intracellular delivery peptides having the ability to rapidly transduce polypeptide cargoes to the cytosolic/nuclear compartment of eukaryotic cells. In contrast to traditional cell penetrating peptide-based intracellular delivery strategies, synthetic peptide shuttle agents are independent from, or are not covalently linked to, their polypeptide cargoes at the moment of transduction across the plasma membrane. In fact, covalently linking shuttle agents to their cargoes in an uncleavable manner generally has a negative effect on their transduction activity.

The first generation of synthetic peptide shuttle agents was described in WO/2016/161516 and consisted of multi-domain-based peptides having an endosome leakage domain (ELD) operably linked to a cell penetrating domain (CPD), and optionally further comprising one or more histidine-rich domains. Although it was initially believed that shuttle agent-mediated cargo transduction occurred via mechanisms similar to that of conventional cell-penetrating peptides, the speed and efficiency of cargo delivery to the cytosolic/nuclear compartment suggested a strong contribution from a more direct delivery mechanism across the plasma membrane without requiring complete endosomal formation (Del'Guidice et al., 2018). Therefore, using the first-generation shuttle agents as a starting point, a large scale iterative design and screening program was undertaken to optimize the shuttle agents for the rapid and efficient transduction of polypeptide cargoes while reducing cellular toxicity. The program involved the manual and computer-assisted design/modeling of almost 11,000 synthetic peptides, as well as the synthesis and testing of several hundred different peptides for their ability to transduce a variety of polypeptide cargoes rapidly and efficiently in a plurality of cells and tissues. Rather than considering the shuttle agents as fusions of known cell-penetrating peptides (CPDs) and endosomolytic peptides (ELDs) derived from the literature, each peptide was considered holistically based on their predicted three-dimensional structure and physicochemical properties. The design and screening program culminated in a second generation of synthetic peptide shuttle agents defined by a set of fifteen parameters described in WO/2018/068135 governing the rational design of shuttle agents with improved transduction/toxicity profiles for polypeptide cargoes over the first generation shuttle agents. These second generation synthetic peptide shuttle agents were designed and empirically screened for the rapid transduction of polypeptide cargoes (i.e., typically within under 5 minutes) and thus were predominantly designed to lack a prototypical CPD.

### Example 3: Truncated Synthetic Peptide Shuttle Agents Retain Transduction Activity

Shuttle agent truncation experiments were undertaken to identify minimal fragments of first- and second-generation synthetic peptide shuttle agents sufficient for cargo transduction activity. These experiments revealed that C-terminal truncations were generally more tolerated than N-terminal truncations, with C-terminal truncations often retaining substantial cargo transduction activity when the N-terminal fragment was predicted to adopt an amphipathic cationic alpha helical structure when in solution at physiological conditions.

To test the transduction activity of short/truncated synthetic shuttle agents (e.g., generally having less than 20 amino acids), HeLa cells were incubated with control peptides and N-terminal shuttle agent fragments of different lengths and delivery of GFP or PI was assessed by flow cytometry as described in Example 1. The results shown in FIG. 1 rank the delivery of GFP and PI by each shuttle agent or controls (non-treated [NT] and GFP/PI only [no shuttle agent]) and are ranked according to their “Overall Delivery Factor”. The Overall Delivery Factor represents a single number that accounts for the toxicity of each shuttle agent/peptide, as well as its ability to deliver GFP and PI, and was calculated as follows:

$$\frac{( mean PI delivery score ) \times ( mean viability [ PI ] ) + ( mean GFP delivery score ) \times ( mean viability [ GFP ] )}{2}$$

Shuttle agents having an Overall Delivery Factor greater than 0.5 possessed generally common characteristics (FIG. 1). Typically, these shuttle agents had a hydrophobic moment (μH) of at least 4. Furthermore, when projected into a Schiffer-Edmundson's wheel representation (helical wheel projection) depicting an amphipathic alpha-helical motif, the shuttle agents possessed hydrophobic and positively charged outer surfaces bearing particular angles and a certain percentage of specific residues. According to a typical Schiffer-Edmundson's wheel representation of 18 amino acids, the angle between two consecutive amino acids is 20 degrees, as described in Schiffer et al., 1967. Here, we determine the hydrophobic angle by first determining a region or cluster rich in hydrophobic amino acids and multiply 20 degrees by the number of spaces between each consecutive amino acid in the region or cluster. Similarly, the positively charged angle is calculated by first determining a region or cluster rich in the positively charged residues lysine (K) and arginine (R). The K and R residues most often consecutively appear, but the region or cluster may also comprise weakly or non-hydrophobic residues. In most cases, effective shuttle agents had a positively charged region defined by an angle between 60 and 120 degrees comprised of over 50% lysine (K) and/or arginine (R) residues. The larger hydrophobic angle of these shuttle agents were mostly defined between 180 and 240 degrees and comprised of over 50% phenylalanine (F), isoleucine (I), leucine (L) and/or tryptophan (W). Similar observations were made for shuttle agents longer than 20 amino acids comprising linker sequences or histidine-rich domains. Interestingly, transduction activity was observed for the CM18 peptide (18 amino acids long) in this experiment, which is an N-terminal fragment (endosome leakage domain) in some first generation shuttle agents. Computer-generated 3D images of the peptides of FIG. 1 are shown in core and side views in FIG. 9.

### Example 4: Inhibition of Shuttle Agent Transduction Activity by Cas9-RNP Complexes not Alleviated by Coating with Charge-Neutralizing Agents

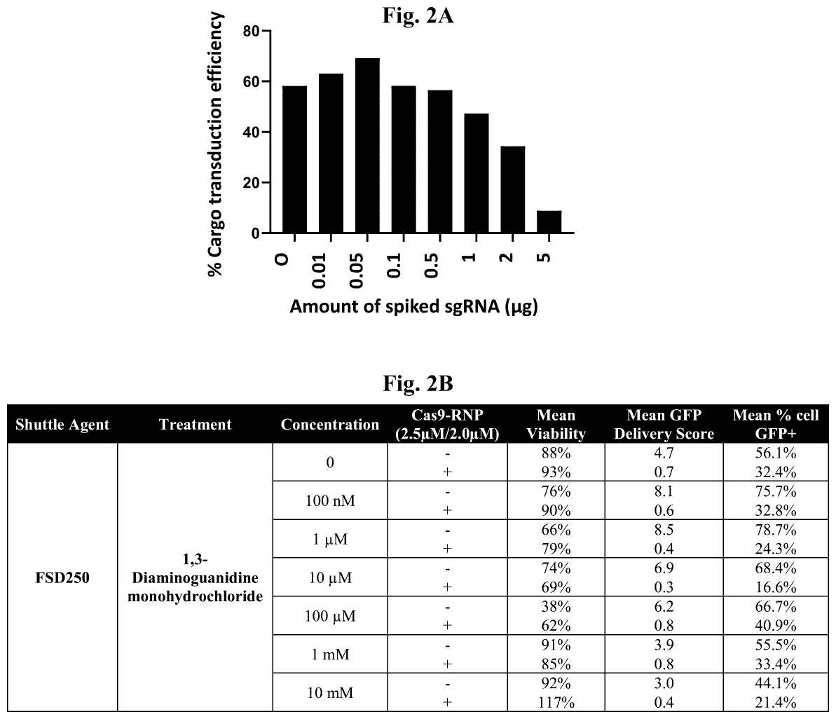

Nuclear delivery of Cas9-sgRNA complexes (hereinafter Cas9-RNP) via first- and second-generation synthetic peptide shuttle agents generally occurs less efficiently than delivery of a Cas9 proteinaceous cargo alone (i.e., without its corresponding sgRNA; see Krishnamurthy et al., 2019, supplementary FIG. 6). The negative effect of the sgRNA (without Cas9) on shuttle agent-mediated transduction of a fluorescently-labelled charge-neutral polynucleotide analog cargo (phosphorodiamidate morpholino oligomer (PMO)) is also shown in FIG. 2A. Briefly, RH-30 cells (150,000 cells/well in 24-well dish) were contacted with a delivery mix containing 6 μM of PMO-FITC and 5 μM of the synthetic peptide shuttle agent FSD250 for 2 minutes in RPMI, in the presence of increasing amounts of sgRNA spiked in the medium. Cells were then washed, incubated in complete medium and then collected for analysis by flow cytometry after 1 h. The results in FIG. 2A show that reduced cargo transduction efficiency was observed in the presence of 2 μg of sgRNA (4 μg/mL).

Hypothesizing that the inhibitory effect of the sgRNA was due to the negatively charged phosphate backbone of the RNA, we attempted to neutralize the negative charges by coating the Cas9-RNP complexes with small positively charged molecules prior to transduction. Delivery of GFP in HeLa cells in the presence of Cas9-RNP was assessed in the presence of small positively charged molecules such as 1,3-diaminoguanidine monohydrochloride; 3,5-diamino-1,2,4-triazole; guanidine hydrochloride; or L-arginine amide dihydrochloride. As shown in FIG. 2B, the presence of Cas9-RNP significantly inhibited both GFP transduction activity (“Mean % cells GFP+”) and Mean GFP delivery Scores, despite the presence of up to 10 mM of 1,3-diaminoguanidine monohydrochloride. Similar results were seen in the presence of 3,5-diamino-1,2,4-triazole; guanidine hydrochloride; or L-arginine amide dihydrochloride (data not shown).

### Example 5: Shuttle Agents Having Increased Resistance to Inhibition by Cas9-RNP

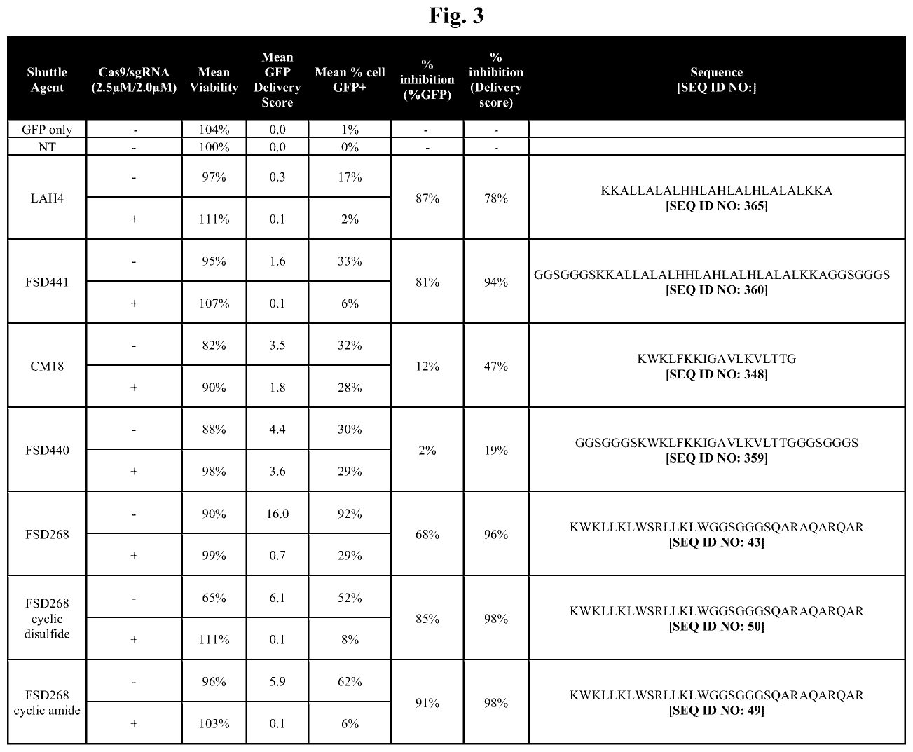

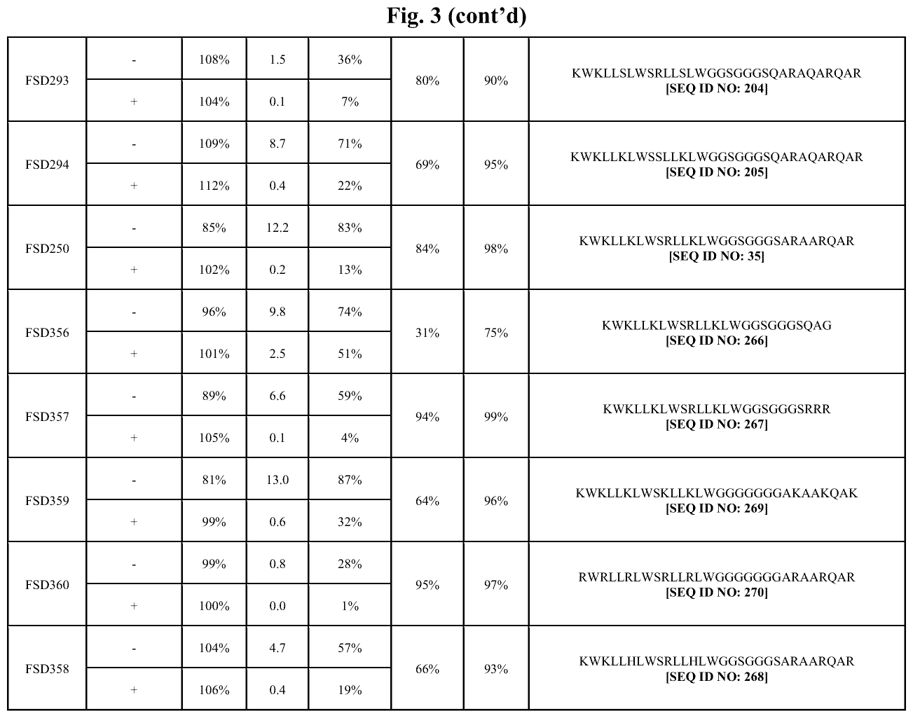

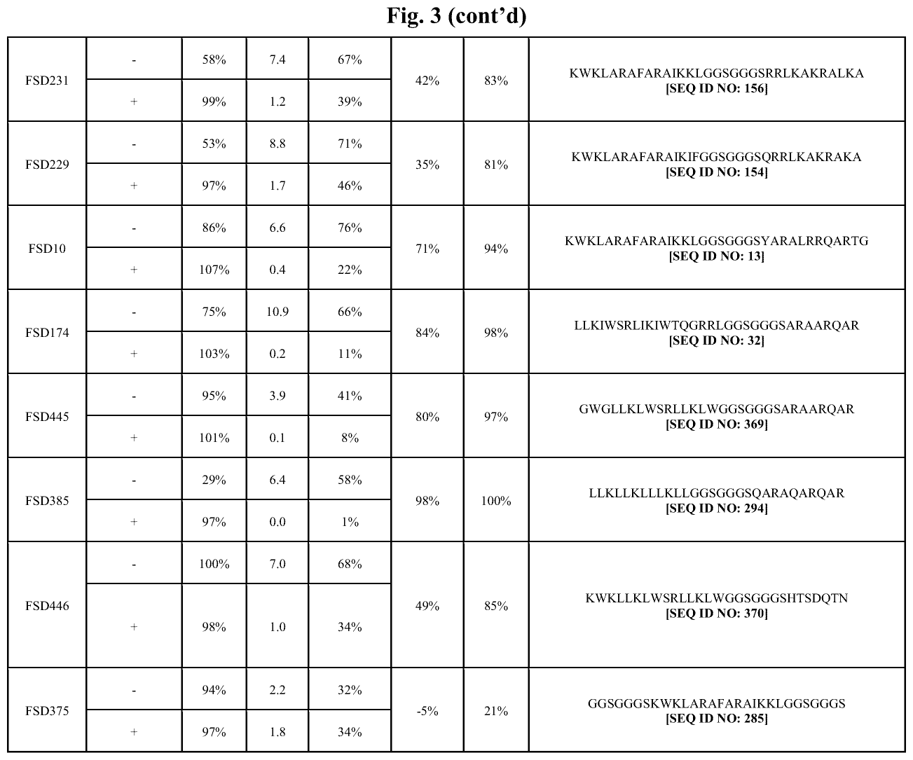

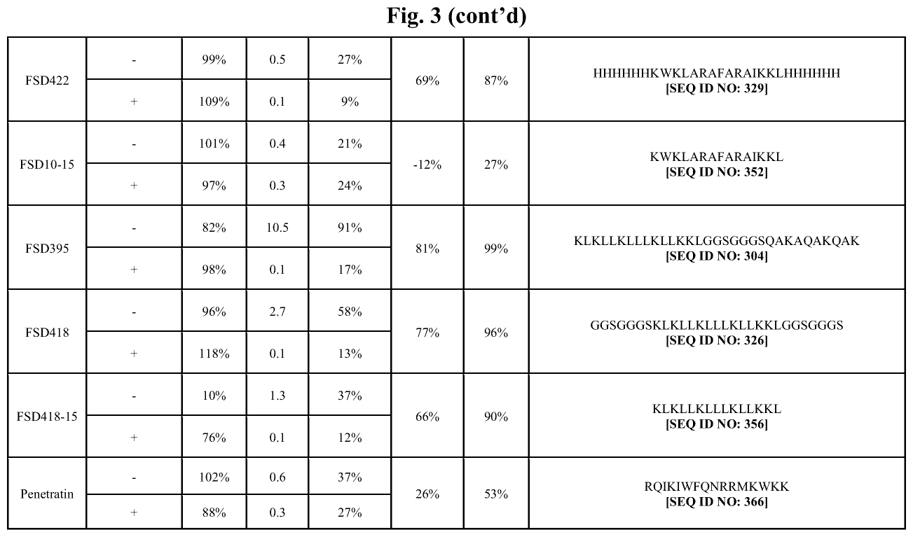

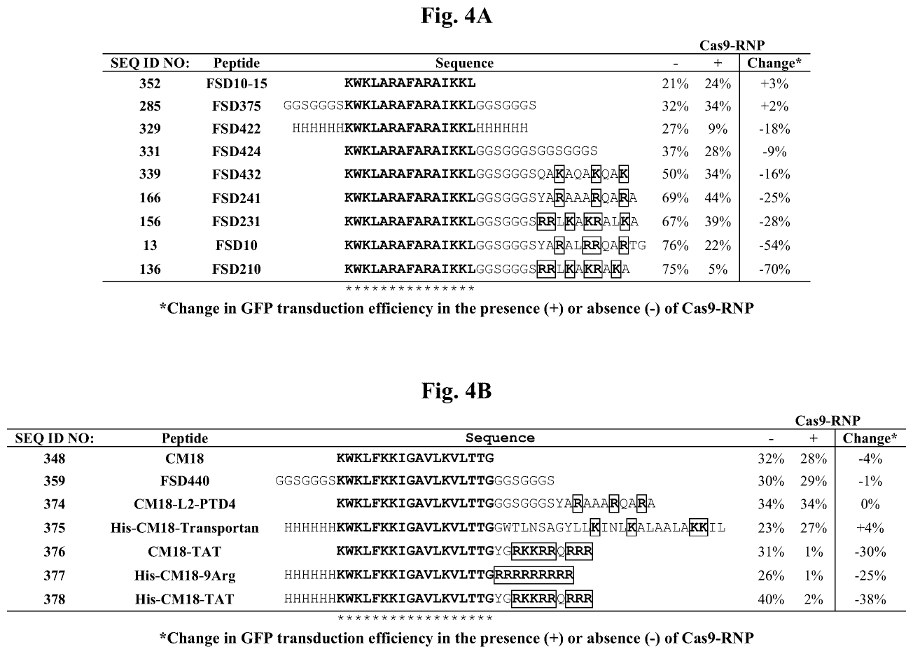

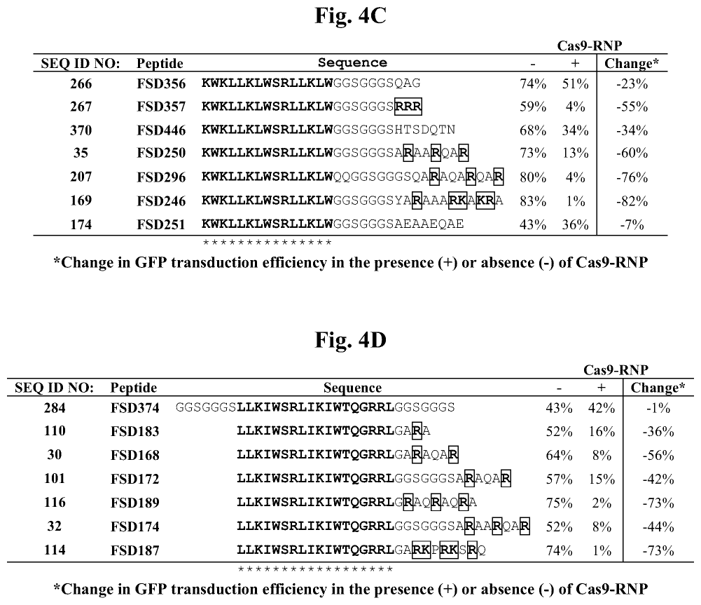

To better understand the inhibitory effect of Cas9-RNP on shuttle agent transduction activity, HeLa cells were incubated with different peptides/shuttle agents and GFP cargo, in the presence or absence of Cas9-RNP, and delivery of GFP was assessed by flow cytometry, as described in Example 1. As shown in FIG. 3, the presence of Cas9-RNP decreased the transduction efficiency of the GFP cargo for the majority of shuttle agents. For some shuttle agents, the effect was particularly striking. For example, the GFP transduction efficiency for FSD268 decreased from 92% to 29%, the GFP transduction efficiency for FSD250 decreased from 83% to 13%, the GFP transduction efficiency for FSD10 decreased from 76% to 22%, and the GFP transduction efficiency for FSD395 decreased from 91% to 17%. A subset of shuttle agents, however, showed a degree of resistance to the negative effects of Cas9-RNP. These more resistant peptides included FSD10-15, CM18, and FSD356. Structure-activity relationships were explored further by repeating the above transduction experiments with shuttle agent variants sharing the same “core” amphipathic cationic alpha helical region as FSD10-15 (FIG. 4A), CM18 (FIG. 4B), and FSD356 (FIG. 4C).

For FSD10-15, GFP transduction efficiency slightly increased from 21% to 24% in the presence of Cas9-RNP (FIG. 3). Interestingly, FSD10-15 is a 15-amino acid fragment of several longer shuttle agents, including FSD375, FSD422, FSD424, FSD432, FSD241, FSD231, FSD10, and FSD210. As shown in FIG. 4A, adding flanking glycine/serine-rich residues to FSD10-15 (see FSD375 and FSD424) retained the peptide's resistance to Cas9-RNP while improving GFP transduction activity over FSD10-15. Replacing the glycine/serine-rich residues with flanking histidine residues (see FSD422) did not maintain the same level of Cas9-RNP resistance. Of note, histidine-rich domains are capable of becoming increasingly cationic at pH values approaching the pKa of their imidazole side chains (about 6). Finally, the presence of a C-terminal glycine/serine-rich linker fused to a second cationic domain (FSD432, FSD241, FSD231, FSD10, and FSD210) seemed to render the shuttle agents more sensitive to inhibition by Cas9-RNP, with the effect FSD10 and FSD210 being particularly pronounced. FSD231 differs from FSD210 only by the insertion of a single leucine residue (L) immediately preceding the most C-terminal lysine residue (K), thereby decreasing the C-terminal positive charge density of FSD231 relative to FSD210. Interestingly, the insertion of this hydrophobic leucine residue rendered FSD231 substantially more resistant to inhibition by Cas9-RNP as compared to FSD210 (FIG. 4A).

For CM18, GFP transduction efficiency remained similar in the absence (32%) and presence (28%) of Cas9-sgRNA (FIG. 3). As shown in FIGS. 3 and 4B, adding flanking glycine/serine-rich residues to CM18 (see FSD440) retained the peptide's resistance to Cas9-RNP but GFP delivery score was increased by two-fold (1.8 for CM18 to 3.6 for FSD440). Shuttle agents having high C-terminal positive charge densities (e.g., CM18-TAT, His-CM18-9Arg, and His-CM18-TAT) exhibited particularly marked sensitivities to inhibition by Cas9-RNP (FIG. 4B), while shuttle agents with lower C-terminal positive charge densities (e.g., CM18-L2-PTD4 and His-CM18-Transportan) were substantially more resistant to inhibition by Cas9-RNP (FIG. 4B). The results in FIG. 4B also suggest that the presence of hydrophobic residues (e.g., A, L, and I) interspaced between C-terminal positively charged residues are advantageous for Cas9-RNP resistance, as well as distancing the C-terminal positively charged residues from the “core” amphipathic cationic alpha helical region (e.g., CM18). Amongst all peptides tested in FIG. 3, FSD356 exhibited the highest GFP transduction efficiency (51%) in the presence of Cas9-RNP. The N-terminal segment of FSD356 is identical to that of FSD446, FSD357, FSD250, FSD296, FSD246, and FSD251. As shown in FIG. 4C, replacing the last three C-terminal residues of FSD356 (QAG) with three positively-charged arginine residues (RRR; see FSD357) resulted in a striking reduction in GFP transduction efficiency in the presence of Cas9-RNP—i.e., from 51% for FSD356 to a mere 4% for FSD357. In contrast, replacing the C-terminal RRR residues in FSD357 by non-cationic hydrophilic residues, including a negatively-charged aspartate (D) group, brought the GFP transduction efficiency in the presence of Cas9-RNP back up to 34%. Finally, replacing the C-terminal non-cationic hydrophilic segment of FSD446 with a cationic segment (see FSD250) resulted in a drop in GFP transduction efficiency in the presence of Cas9-RNP down to 13%. Insertion of polar residues (e.g., Q) between the C-terminal positively-charged residues (e.g., FSD296) and/or increasing C-terminal positive charge density (e.g., FSD246) increased shuttle agent sensitivity to inhibition by Cas9-RNP (FIG. 4C). Strikingly, the shuttle agent FSD251, which contains three negatively-charged glutamate (E) residues in the peptide's C-terminal region, exhibited the least sensitivity to Cas9-RNP inhibition amongst the variants compared in FIG. 4C.

Of the peptides tested in FIG. 3, FSD174 was among those particularly sensitive to the inhibitory effect of Cas9-RNP, with its GFP transduction efficiency decreasing from 66% to 11% in the presence of Cas9-RNP. Structure-activity relationships relating to this inhibition were explored by repeating the above transduction experiments with shuttle agent variants sharing the same “core” amphipathic cationic alpha helical region as FSD174. As shown in FIG. 4D, shuttle agents having higher C-terminal positive charge densities (e.g., FSD189, FSD 174 and FSD187) were generally more sensitive to Cas9-RNP inhibition than shuttle agents having lower C-terminal positive charge densities. A comparison of the transduction efficiencies and structures of FSD168 vs FSD172, and FSD189 vs FSD 174, suggests that the presence of glycine/serine-rich residues increasing the distance of the C-terminal cationic residues from the N-terminal “core” amphipathic cationic alpha helical region may be advantageous for increased resistance to Cas9-RNP inhibition. Strikingly, FSD374 exhibited virtually the same GFP transduction efficiency in the presence or absence of Cas9-RNP (FIG. 4D). These results, which mirror those observed for FSD375 in FIG. 4A, suggest that flanking the “core” amphipathic cationic alpha helical region with glycine/serine-rich residues is advantageous for resistance to Cas9-RNP inhibition.

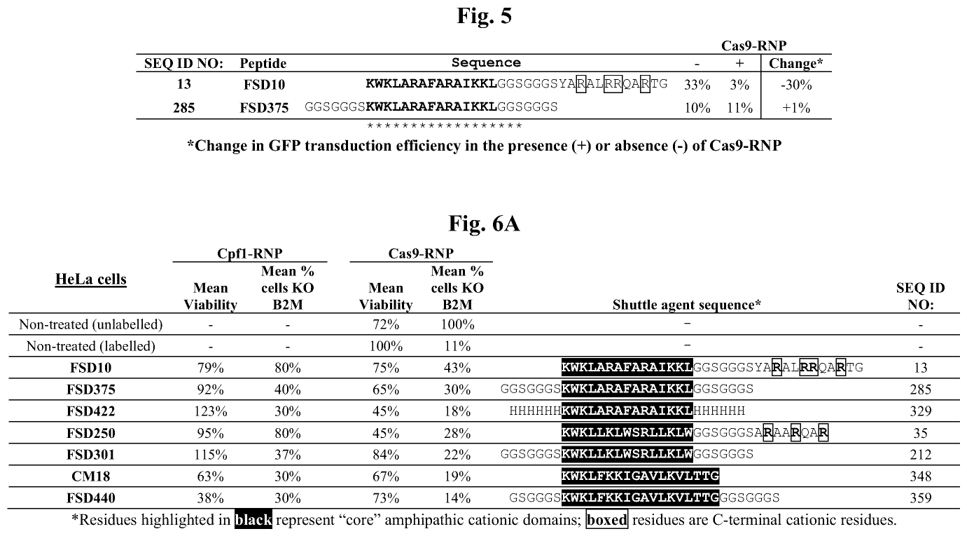

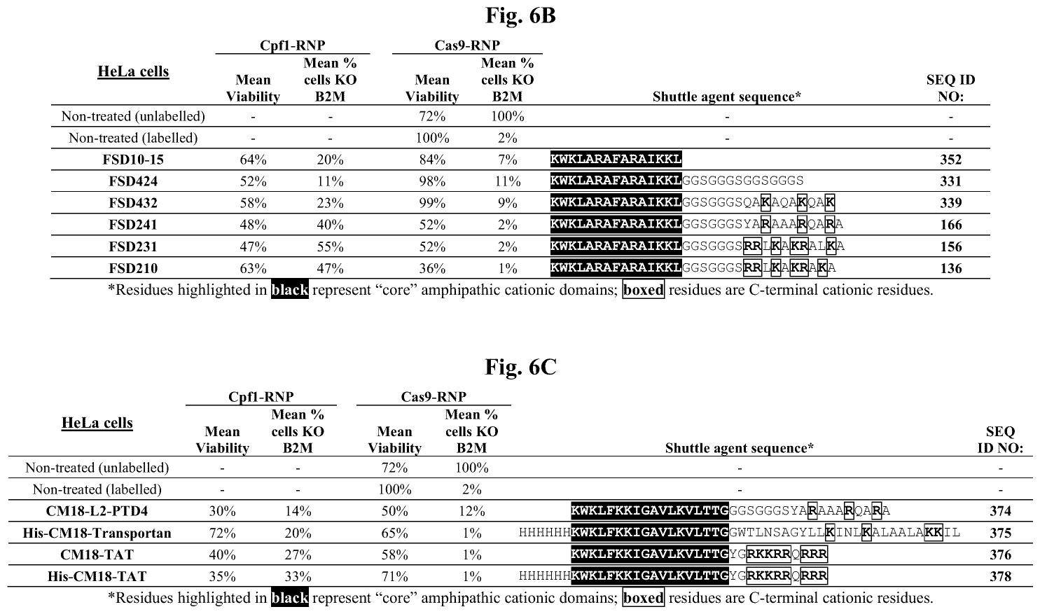

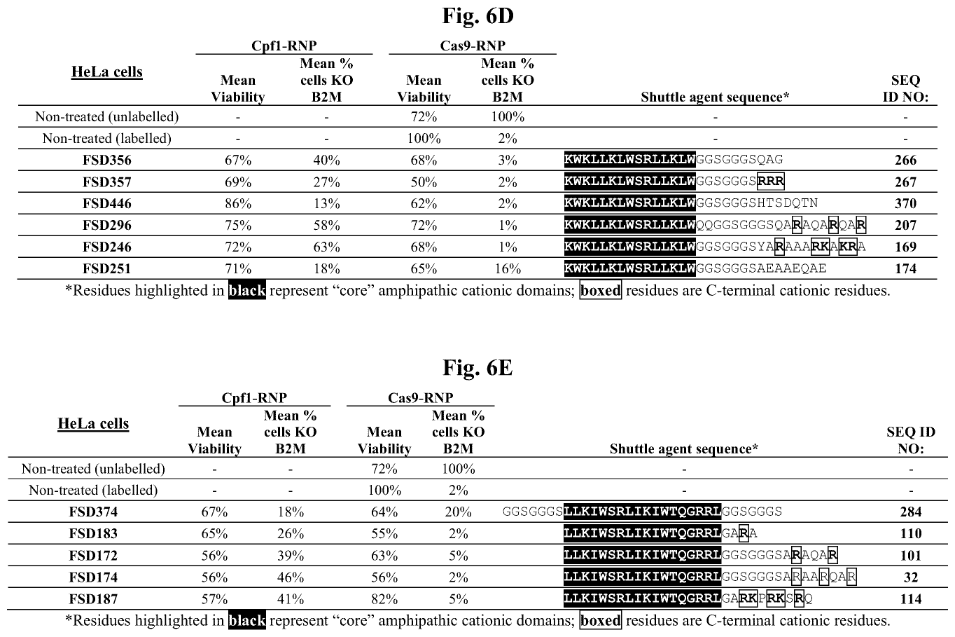

The above transduction experiments were repeated with FSD10 and FSD375 in a Human Bronchial Epithelial cell line model of cystic fibrosis, CFF-16HBEge. As shown in FIG. 5, FSD375 exhibited greater resistance to Cas9-RNP inhibition than FSD10 in CFF-16HBEge cells as well.

Overall, the structure-function studies in this Example strongly suggest that reducing the cationic charge density (i.e., K/R residues per peptide segment length) in at least the C-terminal region of shuttle agents, for example by decreasing the number of positively charged residues per peptide segment length and/or interspacing the C-terminal positively charged residues with hydrophobic residues (e.g., A, L, and I), increases their resistance to Cas9-RNP inhibition (FIG. 4 and FIG. 5). Furthermore, FIGS. 4 and 5 show that flanking a core amphiphilic cationic N-terminal segment of longer shuttle agents with non-cationic and/or negatively-charged hydrophilic residues may increase resistance to Cas9-sgRNA inhibition and likely to other nucleoprotein complexes.

### Example 6: Shuttle Agent-Mediated Delivery of Functional Cas9/Cpf1-RNP Complexes in HeLa Cells

The ability of shuttle agents to deliver Cas9-RNP complexes intracellularly was measured indirectly in Example 5 via the complexes' inhibitory effect on co-delivery of GFP. In the present Example, we assessed the ability of shuttle agents to deliver functional CRISPR Cas9-RNP or Cpf1-RNP complexes to the nucleus of target cells by measuring the phenotypic outcomes of successful genome editing. HeLa cells were incubated with different shuttle agents and Cas9-RNP or Cpf1-RNP targeting the gene encoding B2M (β2 microglobulin), as described in Example 1. Six days post-delivery of Cas9-RNP or Cpf1-RNP complexes, genome editing efficiency was assessed by detection of cells lacking B2M expression (B2M knockout) by flow cytometry. FIG. 6A-6E show the results for delivery of functional Cas9- or Cpf1-sgRNA complexes by various synthetic shuttle agents. The results in FIG. 6A-6E generally show that the decrease in Cas9-RNP genome-editing efficiency, as compared to Cpf1-RNP, was generally smaller for shuttle agents having lower C-terminal cationic charge densities and/or having a core amphiphilic cationic segment flanked by one or more non-cationic hydrophilic residues.

### Example 7: Shuttle Agent-Mediated Delivery of Functional Cas9/Cpf1/ABE-Cas9-sgRNA Complexes in a Human Bronchial Epithelial Cell Line Model of Cystic Fibrosis

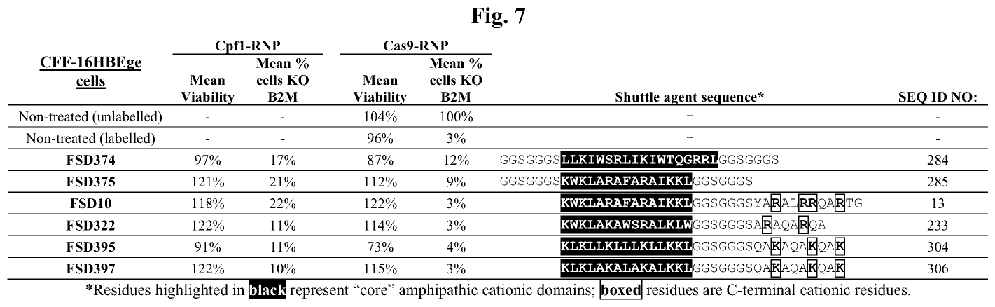

A similar genome editing experiment as in Example 6 was performed in the Human Bronchial Epithelial cell line model of cystic fibrosis, CFF-16HBEge. Genome editing results are shown in FIG. 7. Overall, lower genome editing efficiencies were observed in CFF-16HBEge cells as compared to HeLa cells, consistent with the refractory nature of lung epithelial cells. As seen in the results in HeLa cells in FIG. 6A-6E, shuttle agents transduced Cpf1-RNP complexes better than Cas9-RNP in CFF-16HBEge cells (FIG. 7). The shuttle agents FSD10, FSD322, FSD395, and FSD397 all exhibited greater than 10% genome editing efficiency with Cpf1-RNP, but failed to show any increase in genome editing over the non-treated (labelled) negative control with respect to Cas9-RNP. The only two shuttle agents amongst those tested that exhibited significant genome editing with Cas9-RNP as cargo were FSD374 and FSD375, which have similar structures of a core amphipathic cationic domain flanked by short segments of non-cationic hydrophilic residues.

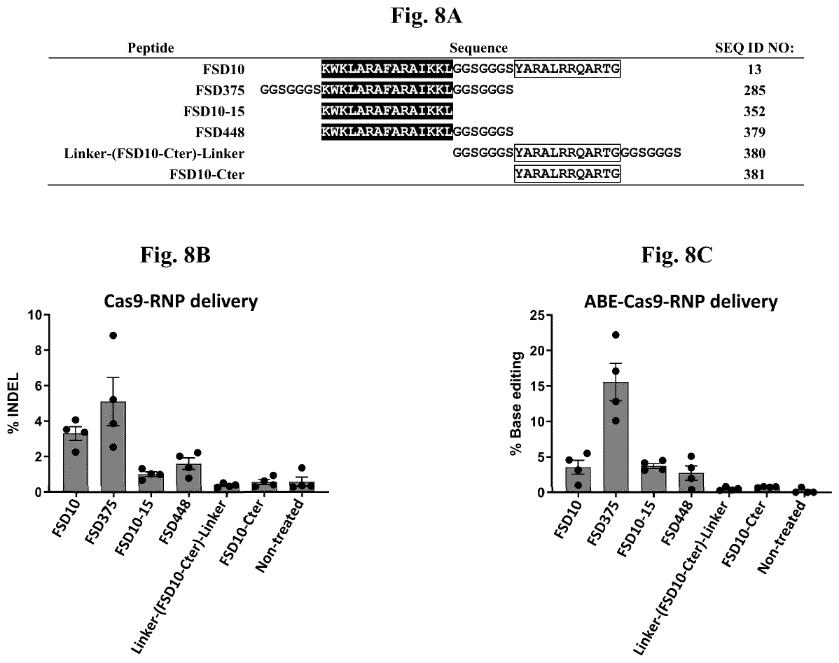

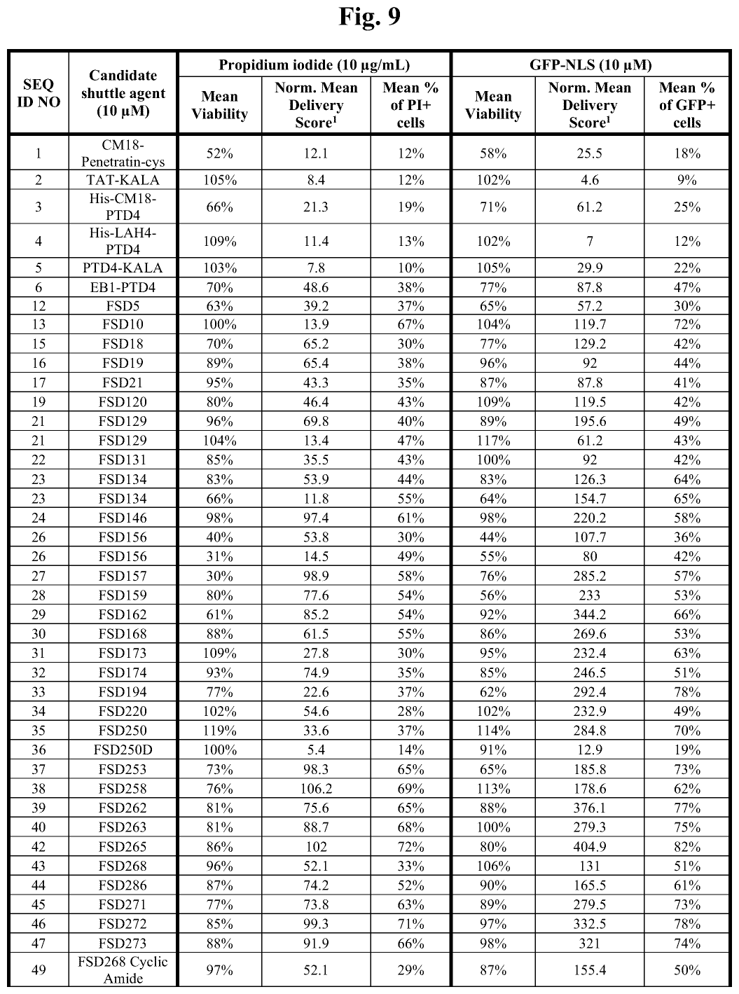

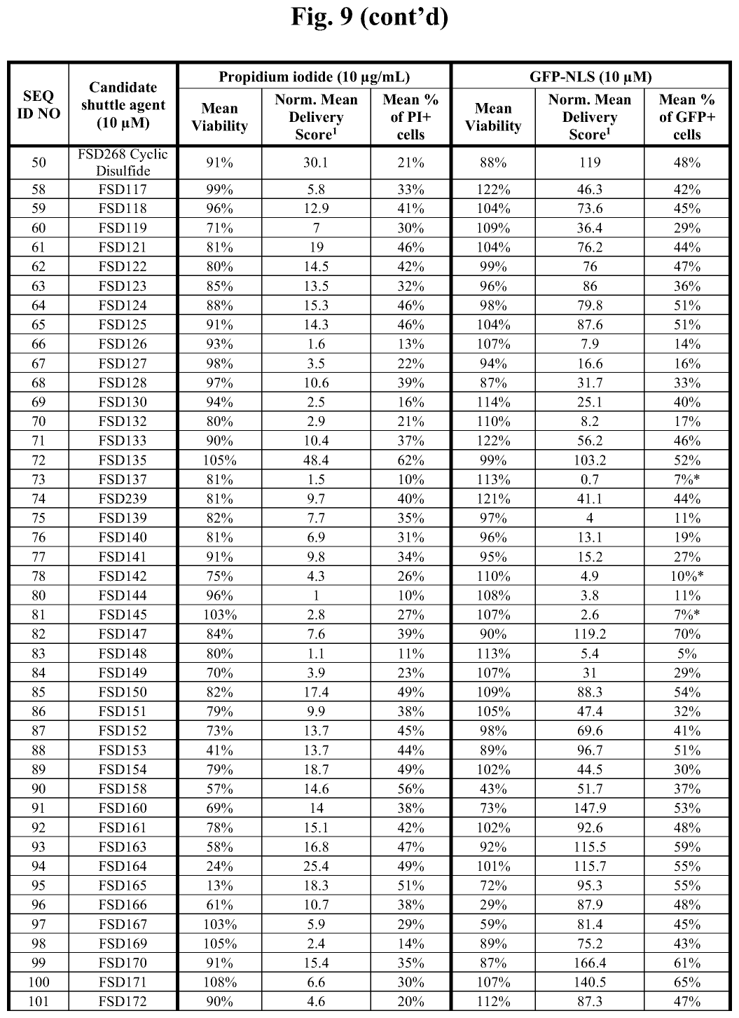

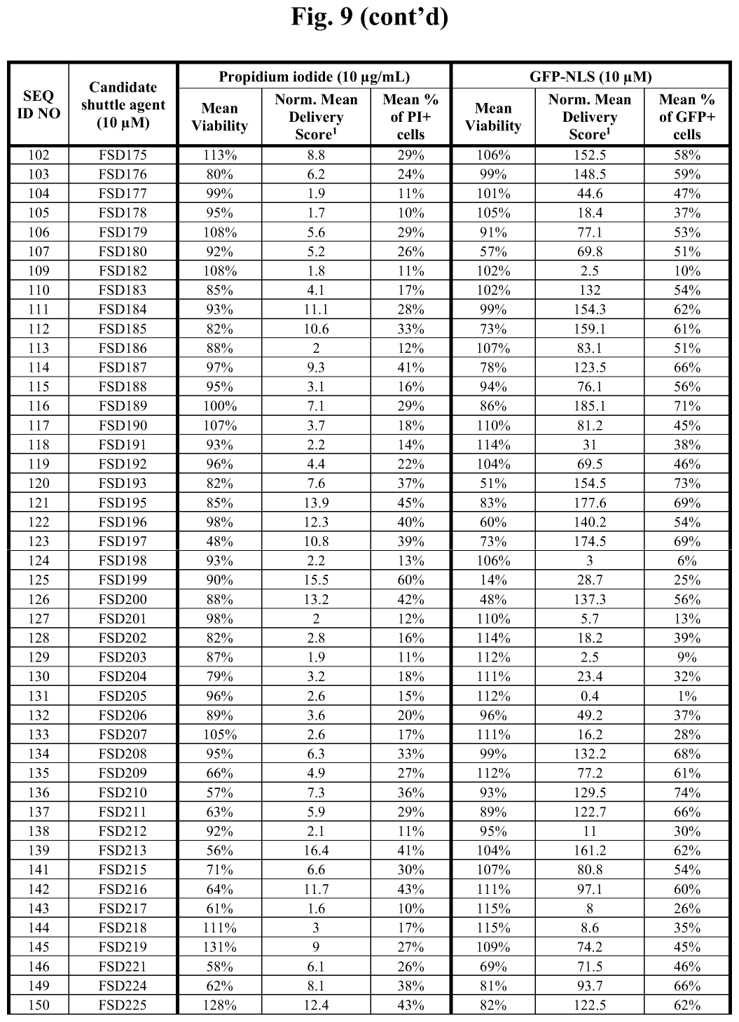

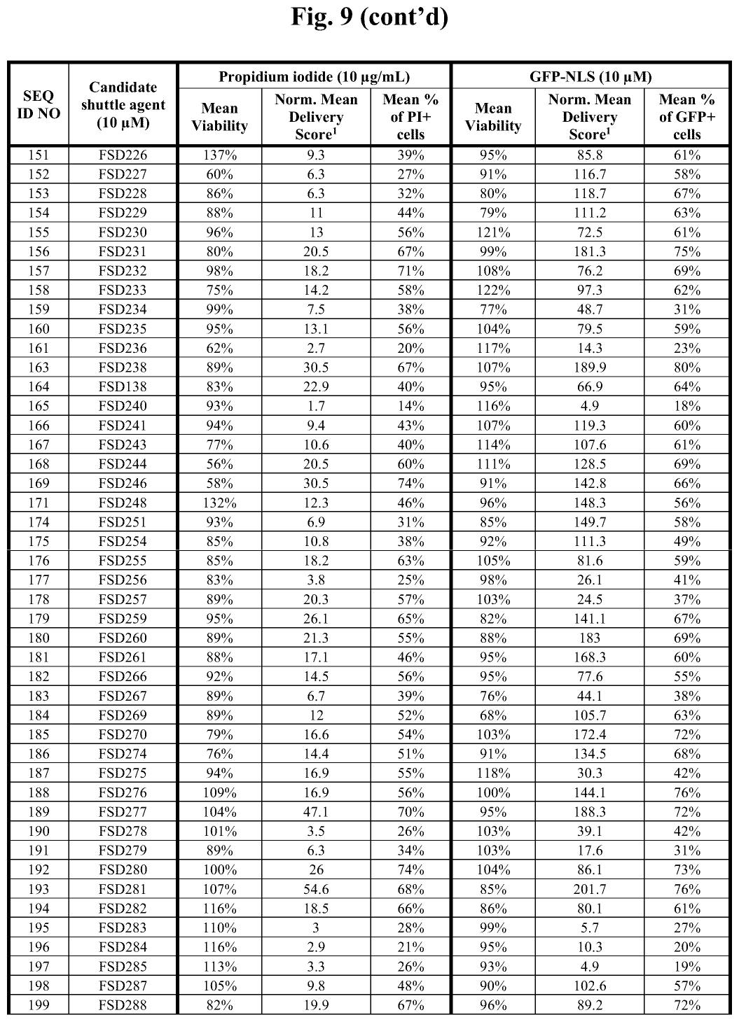

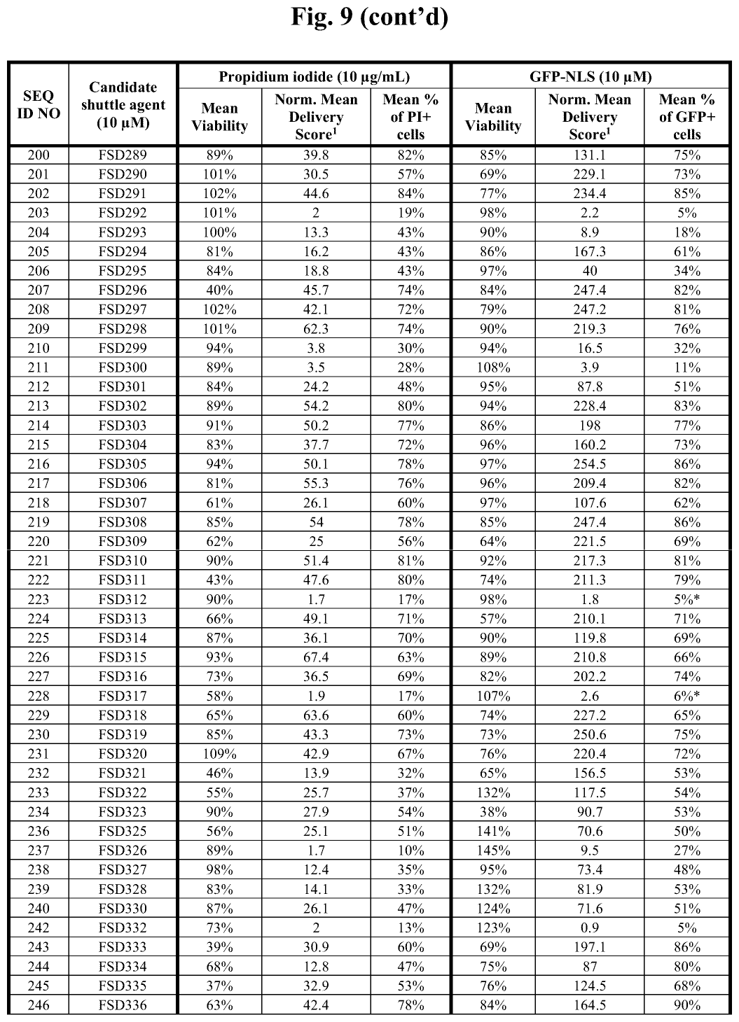

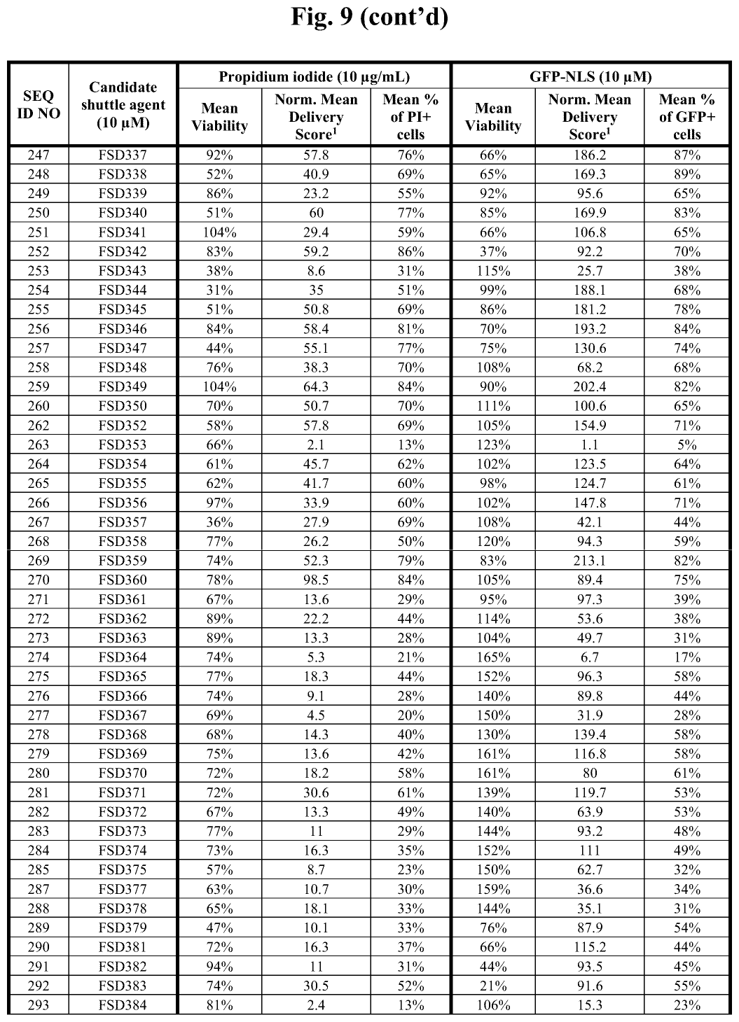

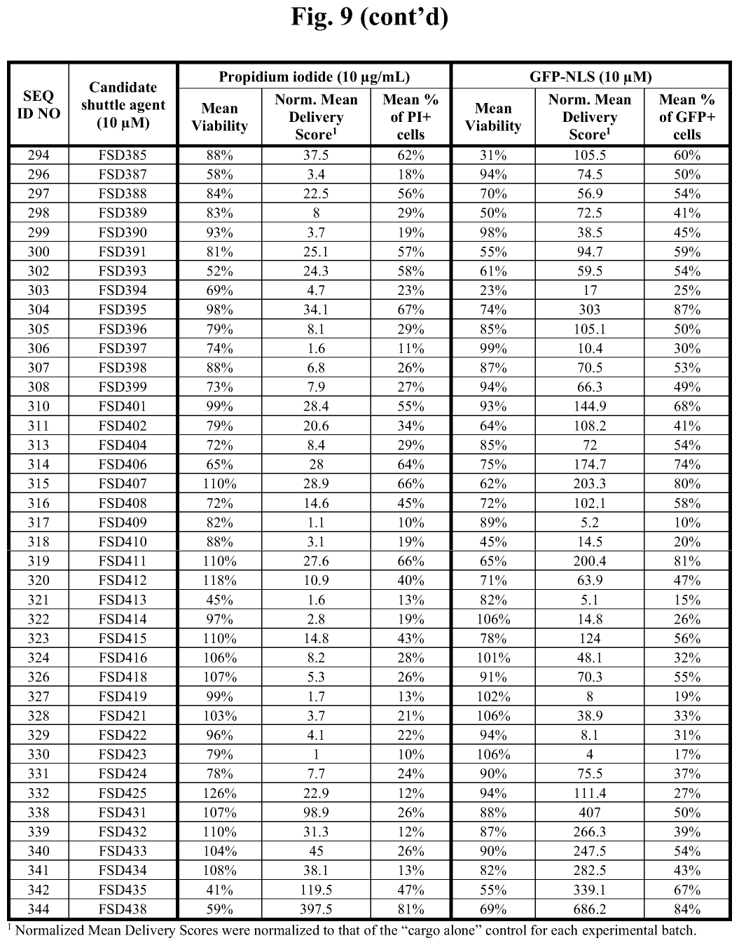

Further transduction experiments in CFF-16HBEge cells were performed to compare the ability of shuttle agents to deliver functional Cas9-RNP genome editing versus ABE-Cas9-RNP base editing complexes. Variants of the shuttle agent FSD10 (FIG. 8A) were tested in parallel, and genome editing/base editing results as evaluated by next-generation sequencing (NGS) are shown in FIGS. 8B and 8C for Cas9-RNP and ABE-Cas9-RNP, respectively. Interestingly, delivery of ABE-Cas9-RNP base editing complex with the shuttle agent FSD375 resulted in significantly higher base editing efficiency (about 15%) over other shuttle agents tested (FSD10, FSD10-15, and FSD448) (FIG. 8C). Negative control peptides consisting of the C-terminal cationic portion of FSD10 alone (“FSD10-Cter”) or flanked glycine/serine-rich linkers (“Linker-(FSD10-Cter)-Linker”) (FIG. 8A) did not exhibit any detectable genome editing or base editing as compared to non-treated cells (FIGS. 8B and 8C).

### Example 8: Large-Scale Screening of Candidate Peptide Shuttle Agents for Propidium Iodide (PI) and GFP-NLS Transduction Activity

A proprietary library of over 300 candidate peptide shuttle agents was screened in parallel for both propidium iodide (PI) and GFP-NLS transduction activity in HeLa cells using flow cytometry as generally described in Example 1. PI was used a cargo because it exhibits 20- to 30-fold enhanced fluorescence and a detectable shift in maximum excitation/emission spectra only after being bound to genomic DNA—a property that makes it particularly suitable to distinguish endosomally-trapped cargo from endosomally-escaped cargo having access to the cytosolic/nuclear compartment. Thus, intracellular delivery and endosomal escape could both be measurable by flow cytometry since any PI that remained trapped in endosomes would not reach the nucleus and would exhibit neither the enhanced fluorescence nor the spectra shift.

Due to the large number of peptides screened, negative controls were performed in parallel for each experimental batch and included a “no treatment” (NT) control in which the cells were not exposed to shuttle peptide or cargo, as well as a “cargo alone” control in which cells were exposed to the cargo in the absence of shuttle agent. Results are shown in FIG. 9, in which “transduction efficiency” refers to the percentage of all viable cells that are positive for the cargo (PI or GFP-NLS). “Mean Delivery score” provides a further indication of the total amount of cargo that was delivered per cell, amongst all cargo-positive cells. Mean PI or GFP-NLS delivery score was calculated by multiplying the mean fluorescence intensity (of at least duplicate samples) measured for the viable PI+ or GFP+ cells by the mean percentage of viable PI+ or GFP+ cells, divided by 100,000 for GFP delivery or by 10,000 for PI delivery. The Mean Delivery Scores for PI and GFP-NLS for each candidate shuttle agent was then normalized by dividing by the Mean Delivery Score for the “cargo alone” negative control performed in parallel for each experimental batch. Thus, the “Norm. Mean Delivery Score” in FIG. 9 represents the fold-increase in Mean Delivery Score over the “cargo alone” negative control.

The batch-to-batch variation observed for the negative controls was relatively small for GFP-NLS but was appreciably higher with PI as cargo. For example, the variation in transduction efficiency for the “cargo alone” negative control ranged from 0.4% to 1.3% for GFP-NLS and from 0.9% to 6.3% for PI. Furthermore, transduction efficiencies for several negative control peptides (i.e., peptides known to have low or no GFP transduction activity) tested in parallel (e.g., FSD174 Scramble; data not shown) sometimes gave lower transduction efficiencies for PI (but not for GFP-NLS) than the “cargo alone” negative control, in some cases by as much as 5%, perhaps due to non-specific interactions between PI and the peptides. This phenomenon was not observed for GFP-NLS transduction experiments. The foregoing suggested that the shuttle agent transduction efficiencies at least for PI may be more appropriately compared to that of a negative control peptide rather than to the “cargo alone” condition.

Included amongst the candidate peptide shuttle agents in FIG. 9 having a mean PI transduction efficiency of at least 20% were peptides having lengths of less than 20 residues: FSD390 (17 aa), FSD367 (19 aa), and FSD366 (18 aa). Also included amongst the candidate peptide shuttle agents having a mean PI transduction efficiency of at least 20% were peptides comprising either non-physiological amino acid analogs (e.g., FSD435, which corresponds to FSD395 except for lysine residues (K) being replaced with L-2,4-diaminobutyric acid residues) or chemical modifications (e.g., FSD438, which corresponds to FSD10 except for an N-terminal octanoic acid modification; FSD436, which corresponds to FSD222 except for phenylalanine residues (F) being replaced with (2-naphthyl)-L-alanine residues; FSD171, which corresponds to FSD168 except having an N-terminal acetyl group and a C-terminal cysteamide group. These results confirm the robustness of the peptide shuttle agent platform technology to tolerate the use of non-physiological amino acids or analogs thereof in place of physiological amino acids and/or chemical modifications.

### REFERENCES

Andreu et al., (1992) “Shortened cecropin A-melittin hybrids. Significant size reduction retains potent antibiotic activity”. FEBS letters 296, 190-194
Amand et al., (2012). “Functionalization with C-terminal cysteine enhances transfection efficiency of cell-penetrating peptides through dimer formation.” Biochem Biophys Res Commun 418(3): 469-474.
Boman et al., (1989) Antibacterial and antimalarial properties of peptides that are cecropin-melittin hybrids. FEBS letters 259, 103-106.
Brock et al., (2018) “Efficient cell delivery mediated by lipid-specific endosomal escape of supercharged branched peptides”. Traffic 19(6):421-435. doi: 10.1111/tra.12566.
Chance et al., (2021) “Observations of, and Insights into, Cystic Fibrosis Mucus Heterogeneity in the Pre-Modulator Era: Sputum Characteristics, DNA and Glycoprotein Content, and Solubilization Time”. Journal of Respiration. 1(1), 8-29; https://doi.org/10.3390/jor1010002.
Del'Guidice et al., (2018) “Membrane permeabilizing amphiphilic peptide delivers recombinant transcription factor and CRISPR-Cas9/Cpf1 ribonucleoproteins in hard-to-modify cells”. PLoS ONE 13(4): e0195558.
Drin et al., (2003). “Studies on the internalization mechanism of cationic cell-penetrating peptides.” J Biol Chem 278(33): 31192-31201.
Eisenberg et al., (1982). “The helical hydrophobic moment: a measure of the amphiphilicity of a helix”. Nature 299, 371-374.
El-Andaloussi et al., (2007). “A novel cell-penetrating peptide, M918, for efficient delivery of proteins and peptide nucleic acids.” Mol Ther 15(10): 1820-1826.
El-Sayed et al., (2009). “Delivery of macromolecules using arginine-rich cell-penetrating peptides: ways to overcome endosomal entrapment.” AAPS J 11(1): 13-22.
Elmquist et al., (2001). “VE-cadherin-derived cell-penetrating peptide, pVEC, with carrier functions.” Exp Cell Res 269(2): 237-244.
Erazo-Oliveras et al., (2014) “Protein delivery into live cells by incubation with an endosomolytic agent.” Nat Methods. (8):861-7.
Fawell et al., (1994). “Tat-mediated delivery of heterologous proteins into cells.” Proc Natl Acad Sci USA 91(2): 664-668.
Fominaya et al., (1998). “A chimeric fusion protein containing transforming growth factor-alpha mediates gene transfer via binding to the EGF receptor.” Gene Ther 5(4): 521-530.
Fominaya, J. and W. Wels (1996). “Target cell-specific DNA transfer mediated by a chimeric multidomain protein. Novel non-viral gene delivery system.” J Biol Chem 271(18): 10560-10568.
Glover et al., (2009). “Multifunctional protein nanocarriers for targeted nuclear gene delivery in nondividing cells.” FASEB J 23(9): 2996-3006.
Gottschalk et al., (1996). “A novel DNA-peptide complex for efficient gene transfer and expression in mammalian cells.” Gene Ther 3(5): 448-457.
Green, M. and P. M. Loewenstein (1988). “Autonomous functional domains of chemically synthesized human immunodeficiency virus tat trans-activator protein.” Cell 55(6): 1179-1188.
Hallbrink et al., (2001). “Cargo delivery kinetics of cell-penetrating peptides.” Biochim Biophys Acta 1515(2): 101-109.
Herce, H. D. and A. E. Garcia (2007). “Molecular dynamics simulations suggest a mechanism for translocation of the HIV-1 TAT peptide across lipid membranes.” Proc Natl Acad Sci USA 104(52): 20805-20810.
Ho et al., (2001). “Synthetic protein transduction domains. enhanced transduction potential in vivo.” Cancer Research 61: 474-477.
Ilfeld and Yaksh (2009). “The End of Postoperative Pain—A Fast-Approaching Possibility? And, if So, Will We Be Ready?” Regional Anesthesia and Pain Medicine 34(2): 85-87.
Kakudo et al., (2004). “Transferrin-modified liposomes equipped with a pH-sensitive fusogenic peptide: an artificial viral-like delivery system.” Biochemistry 43(19): 5618-5628.
Kichler et al., (2006). “Cationic amphipathic histidine-rich peptides for gene delivery.” Biochim Biophys Acta 1758(3): 301-307.
Kichler et al., (2003). “Histidine-rich amphipathic peptide antibiotics promote efficient delivery of DNA into mammalian cells”. Proc Natl Acad Sci USA. 2003 Feb. 18; 100(4): 1564-1568.
Krishnamurthy et al., (2019). “Engineered amphiphilic peptides enable delivery of proteins and CRISPR-associated nucleases to airway epithelia”. Nature Communications. 10(1): 4906. doi: 10.1038/s41467-019-12922-y.
Kwon, et al., (2010). “A Truncated HGP Peptide Sequence That Retains Endosomolytic Activity and Improves Gene Delivery Efficiencies”. Mol. Pharmaceutics, 7:1260-65.
Lamiable et al., (2016). “PEP-FOLD3: faster de novo structure prediction for linear peptides in solution and in complex” Nucleic Acids Res. 44(W1):W449-54.
Li et al., (2004). “GALA: a designed synthetic pH-responsive amphipathic peptide with applications in drug and gene delivery.” Adv Drug Deliv Rev 56(7): 967-985.
London, E. (1992). “Diphtheria toxin: membrane interaction and membrane translocation.” Biochim Biophys Acta 1113(1): 25-51.
Lorieau et al., (2010). “The complete influenza hemagglutinin fusion domain adopts a tight helical hairpin arrangement at the lipid:water interface.” Proc Natl Acad Sci USA 107(25): 11341-11346.
Luan et al., (2015). “Peptide amphiphiles with multifunctional fragments promoting cellular uptake and endosomal escape as efficient gene vectors.” J. Mater. Chem. B, 3: 1068-1078.
Mahlum et al., (2007). “Engineering a noncarrier to a highly efficient carrier peptide for noncovalently delivering biologically active proteins into human cells.” Anal Biochem 365(2): 215-221.
Midoux et al., (1998). “Membrane permeabilization and efficient gene transfer by a peptide containing several histidines.” Bioconjug Chem 9(2): 260-267.
Montrose et al., (2013). “Xentry, a new class of cell-penetrating peptide uniquely equipped for delivery of drugs.” Sci Rep 3: 1661.
Morris, M. C., L. Chaloin, M. Choob, J. Archdeacon, F. Heitz and G. Divita (2004). “Combination of a new generation of PNAs with a peptide-based carrier enables efficient targeting of cell cycle progression.” Gene Ther 11(9): 757-764.
Morris et al., (2001). “A peptide carrier for the delivery of biologically active proteins into mammalian cells.” Nat Biotechnol 19(12): 1173-1176.
O'Keefe, D. O. (1992). “Characterization of a full-length, active-site mutant of diphtheria toxin.” Arch Biochem Biophys 296(2): 678-684.
Parente et al., (1990). “Mechanism of leakage of phospholipid vesicle contents induced by the peptide GALA.” Biochemistry 29(37): 8720-8728.
Perez et al., (1992). “Antennapedia homeobox as a signal for the cellular internalization and nuclear addressing of a small exogenous peptide.” J Cell Sci 102 (Pt 4): 717-722.
Salomone et al., (2012). “A novel chimeric cell-penetrating peptide with membrane-disruptive properties for efficient endosomal escape.” J Control Release 163(3): 293-303.
Schiffer et al., (1967). “Use of helical wheels to represent the structures of proteins and to identify segments with helical potential.” Biophysical Journal 7: 121-135.
Schuster et al., “Multicomponent DNA carrier with a vesicular stomatitis virus G-peptide greatly enhances liver-targeted gene expression in mice.” Bioconjug Chem 10(6): 1075-1083.
Shaw et al., (2008). “Comparison of protein transduction domains in mediating cell delivery of a secreted CRE protein.” Biochemistry 47(4): 1157-1166.
Shen et al., (2014) “Improved PEP-FOLD approach for peptide and miniprotein structure prediction”. J. Chem. Theor. Comput. 10:4745-4758.
Tan et al., (2012). “Truncated peptides from melittin and its analog with high lytic activity at endosomal pH enhance branched polyethylenimine-mediated gene transfection.” J Gene Med 14(4): 241-250.
Theriault et al., “Differential modulation of Nav1.7 and Nav1.8 channels by antidepressant drugs.” European Journal of Pharmacology (2015) 764: 395-403.
Thévenet et al., “PEP-FOLD: an updated de novo structure prediction server for both linear and disulfide bonded cyclic peptides.” Nucleic Acids Res. 2012. 40, W288-293.
Uherek et al., (1998). “A modular DNA carrier protein based on the structure of diphtheria toxin mediates target cell-specific gene delivery.” J Biol Chem 273(15): 8835-8841.
Varkouhi et al., (2011). “Endosomal escape pathways for delivery of biologicals.” J Control Release 151(3): 220-228.
Veach et al., (2004). “Receptor/transporter-independent targeting of functional peptides across the plasma membrane.” J Biol Chem 279(12): 11425-11431.
Vives et al., (1997). “A truncated HIV-1 Tat protein basic domain rapidly translocates through the plasma membrane and accumulates in the cell nucleus.” J Biol Chem 272(25): 16010-16017.<
Wyman et al., (1997). “Design, synthesis, and characterization of a cationic peptide that binds to nucleic acids and permeabilizes bilayers.” Biochemistry 36(10): 3008-3017.
Zhou et al., (2009). “Generation of induced pluripotent stem cells using recombinant proteins.” Cell Stem Cell 4(5): 381-384.

1. A composition comprising a nucleoprotein cargo for intracellular delivery and a synthetic peptide shuttle agent that is independent from, or is not covalently linked to, said nucleoprotein cargo, the synthetic peptide shuttle agent being a peptide comprising an amphipathic alpha-helical motif having both a positively-charged hydrophilic outer face and a hydrophobic outer face, wherein synthetic peptide shuttle agent increases cytosolic/nuclear delivery of said nucleoprotein cargo in eukaryotic cells as compared to in the absence of the synthetic peptide shuttle agent.

2. The composition of claim 1, wherein the nucleoprotein cargo is a deoxyribonucleoprotein (DNP) or ribonucleoprotein (RNP) cargo.

3. The composition of claim 1 or 2, wherein the nucleoprotein cargo is an RNA-guided nuclease, a Cas nuclease, such as a Cas type I, II, III, IV, V, or VI nuclease, or a variant thereof that lacking nuclease activity, a base editor, or a prime editor, a CRISPR-associated transposase, or a Cas-recombinase (e.g., recCas9).

4. The composition of any one of claims 1 to 3, the nucleoprotein cargo is Cpf1-RNP.

5. The composition of any one of claims 1 to 3, the nucleoprotein cargo is Cas9-RNP.

6. The composition of any one of claims 1 to 5, wherein the nucleoprotein cargo is not covalently linked or pre-complexed with a cell-penetrating peptide.

7. The composition of any one of claims 1 to 6, wherein the shuttle agent is:
(1) a peptide at least 12, 13, 14, 15, 16, 17, 18, 19, or 20 amino acids in length comprising
(2) an amphipathic alpha-helical motif having
(3) a positively-charged hydrophilic outer face, and a hydrophobic outer face,
wherein at least five of the following parameters (4) to (15) are respected:
(4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn;
(5) the peptide has a hydrophobic moment (μ) of 3.5 to 11;
(6) the peptide has a predicted net charge of at least +3 or +4 at physiological pH;
(7) the peptide has an isoelectric point (pI) of 8 to 13;
(8) the peptide is composed of 35% to 65% of any combination of the amino acids: A, C, G, I, L, M, F, P, W, Y, and V;
(9) the peptide is composed of 0% to 30% of any combination of the amino acids: N, Q, S, and T;
(10) the peptide is composed of 35% to 85% of any combination of the amino acids: A, L, K, or R;
(11) the peptide is composed of 15% to 45% of any combination of the amino acids: A and L, provided there being at least 5% of L in the peptide;
(12) the peptide is composed of 20% to 45% of any combination of the amino acids: K and R;
(13) the peptide is composed of 0% to 10% of any combination of the amino acids: D and E;
(14) the difference between the percentage of A and L residues in the peptide (% A+L), and the percentage of K and R residues in the peptide (K+R), is less than or equal to 10%; and
(15) the peptide is composed of 10% to 45% of any combination of the amino acids: Q, Y, W, P, I, S, G, V, F, E, D, C, M, N, T and H.

8. The composition of claim 7, wherein:
(a) the shuttle agent respects at least six, at least seven, at least eight, at least nine, at least ten, at least eleven, or respects all of parameters (4) to (15);
(b) the shuttle agent is a peptide having a minimum length of 12, 13, 14, 15, 16, 17, 18, 19, 20, 21, 22, 23, 24, 25, 26, 27, 28, 29, or 30 amino acids, and a maximum length of 40, 41, 42, 43, 44, 45, 46, 47, 48, 49, 50, 51, 52, 53, 54, 55, 60, 65, 70, 80, 90, 100, 110, 120, 130, 140, or 150 amino acids;
(c) said amphipathic alpha-helical motif has a hydrophobic moment (μ) between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0;
(d) said amphipathic alpha-helical motif comprises a positively-charged hydrophilic outer face comprising: (i) at least two, three, or four adjacent positively-charged K and/or R residues upon helical wheel projection; and/or (ii) a segment of six adjacent residues comprising three to five K and/or R residues upon helical wheel projection, based on an alpha helix having angle of rotation between consecutive amino acids of 100 degrees and/or an alpha-helix having 3.6 residues per turn;
(e) said amphipathic alpha-helical motif comprises a hydrophobic outer face comprising: (i) at least two adjacent L residues upon helical wheel projection; and/or (ii) a segment of ten adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn;
(f) said hydrophobic outer face comprises a highly hydrophobic core consisting of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the shuttle agent;
(g) the shuttle agent has a hydrophobic moment (μ) between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, or 10.5;
(h) the shuttle agent has a predicted net charge of between +3, +4, +5, +6, +7, +8, +9, to +10, +11, +12, +13, +14, or +15;
(i) the shuttle agent has a predicted pI of 10 to 13; or
(j) any combination of (a) to (i).

9. The composition of claim 7 or 8, wherein said shuttle agent respects at least one, at least two, at least three, at least four, at least five, at least six, or all of the following parameters:
(8) the shuttle agent is composed of 36% to 64%, 37% to 63%, 38% to 62%, 39% to 61%, or 40% to 60% of any combination of the amino acids: A, C, G, I, L, M, F, P, W, Y, and V;
(9) the shuttle agent is composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T;
(10) the shuttle agent is composed of 36% to 80%, 37% to 75%, 38% to 70%, 39% to 65%, or 40% to 60% of any combination of the amino acids: A, L, K, or R;
(11) the shuttle agent is composed of 15% to 40%, 20% to 40%, 20 to 35%, or 20 to 30% of any combination of the amino acids: A and L;
(12) the shuttle agent is composed of 20% to 40%, 20 to 35%, or 20 to 30% of any combination of the amino acids: K and R;
(13) the shuttle agent is composed of 5 to 10% of any combination of the amino acids: D and E;
(14) the difference between the percentage of A and L residues in the shuttle agent (% A+L), and the percentage of K and R residues in the shuttle agent (K+R), is less than or equal to 9%, 8%, 7%, 6%, or 5%; and
(15) the shuttle agent is composed of 15 to 40%, 20% to 35%, or 20% to 30% of any combination of the amino acids: Q, Y, W, P, I, S, G, V, F, E, D, C, M, N, T, and H.

10. The composition of any one of claims 1 to 9, wherein said shuttle agent comprises a histidine-rich domain, optionally wherein the histidine-rich domain is:
(i) positioned towards the N terminus and/or towards the C terminus of the shuttle agent;
(ii) is a stretch of at least 3, at least 4, at least 5, or at least 6 amino acids comprising at least 50%, at least 55%, at least 60%, at least 65%, at least 70%, at least 75%, at least 80%, at least 85%, or at least 90% histidine residues; and/or comprises at least 2, at least 3, at least 4, at least 5, at least 6, at least 7, at least 8, or at least 9 consecutive histidine residues; or
(iii) both (i) and (ii).

11. The composition of any one of claims 1 to 10, wherein said shuttle agent comprises a flexible linker domain rich in serine and/or glycine residues (e.g., separating N-terminal and a C-terminal segments of the shuttle agent; or positioned N- and/or C-terminal of a central amphipathic cationic alpha helical domain).

12. The composition of any one of claims 1 to 11, wherein said shuttle agent comprises or consists of the amino acid sequence of:
(a)[X1]-[X2]-[linked]-[X3]-[X4] (Formula 1);
(b)[X1]-[X2]-[linked]-[X4]-[X3] (Formula 2);
(c)[X2]-[X1]-[linked]-[X3]-[X4] (Formula 3);
(d)[X2]-[X1]-[linked]-[X4]-[X3] (Formula 4);
(e)[X3]-[X4]-[linked]-[X1]-[X2] (Formula 5);
(f)[X3]-[X4]-[X1] (Formula 6);
(g)[X4]-[X3]-[linked]-[X1]-[X2] (Formula 7);
(h)[X4]-[X3]-[X1] (Formula 8);
(i)[linker]-[X1]-[X2]-[linker] (Formula 9);
(j)[linker]-[X2]-[X1]-[linker] (Formula 10);
(k)[X1]-[X2]-[linker] (Formula 11);
(l)[X2]-[X1]-[linker] (Formula 12);
(m)[linker]-[X1]-[X2] (Formula 13);
(n)[linker]-[X2]-[X1] (Formula 14);
(o)[X1]-[X2] (Formula 15); or
(p)[X2]-[X1] (Formula 16),
wherein:
[X1] is selected from: 2[Φ]-1[+]-2[Φ]-1[ζ]-1[+]- ; 2[Φ]-1[+]-2[Φ]-2[+]- ; 1[+]- 1[Φ]-1[+]-2[Φ]-1[ζ]-1[+]- ; and 1 [+]-1[Φ]-1[+]-2[Φ]-2 [+]- ;
[X2] is selected from: -2[Φ]-1[+]-2[Φ]-2[ζ]- ; -2 [Φ]- 1[+]-2[Φ]-2 [+]- ; -2[Φ]-1 [+]-2[Φ]-1[+]-1[ζ]-; -2 [Φ]-1 [+]-2[Φ]-1[ζ]-1 [+]- ; -2[Φ]-2[+]-1 [Φ]-2 [+]- ; -2 [Φ]-2 [+]-1 [Φ]-2[ζ]- ; -2[Φ]-2 [+]-1[Φ]-1[+]-1[ζ]- ; and -2 [Φ]-2[+]-1[Φ]-1[ζ]-1[+]- ;
[X3] is selected from: -4[+]-A- ; -3[+]-G-A- ; -3[+]-A-A- ; -2[+]-1[Φ]-1 [+]-A- ; -2[+]-1[Φ]-G-A- ; -2[+]-1[Φ]-A-A- ; or -2[+]-A-1[+]-A; -2[+]-A-G-A; -2[+]-A-A-A- ; -1 [Φ]-3[+]-A- ; -1 [Φ]-2[+]-G-A- ; -1[Φ]-2[+]-A-A- ; -1 [Φ]-1 [+]-1[Φ]-1[+]-A; -1 [Φ]-1 [+]-1[Φ]-G-A; -1[Φ]-1[+]-1 [Φ]-A-A; -1[Φ]-1[+]-A-1 [+]-A; -1 [Φ]-1 [+]-A-G-A; -1 [Φ]-1 [+]-A-A-A; -A-1 [+]-A-1 [+]-A; -A-1[+]-A-G-A; and -A-1[+]-A-A-A;
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1 [ζ]-A-1 [ζ]-A-1[+]; -2 [+]-A-2[+]; -2 [+]-A-1 [+]-A; -2 [+]-A-1[+]-1[Φ]-A-1[+]; - 2[+]-1[ζ]-A-1[+]; -1[+]-1[ζ]-A-1[+]-A ; -1[+]-1[ζ]-A-2[+]; -1 [+]-1[ζ]-A-1[+]-1 [ζ]-A-1[+]; -1[+]-2 [ζ]-A-1 [+]; -1[+]-2[ζ]-2[+]; -1[+]-2[ζ]-1 [+]-A; -1 [+]-2[ζ]-1[+]-1[ζ]-A-1 [+]; -1[+]-2 [ζ]-1 [ζ]-A-1[+]; -3[ζ]-2[+]; -3[ζ]-1[+]-A; -3[ζ]-1[+]-1[ζ]-A-1[+]; -1[ζ]-2A-1[+]-A; -1[ζ]-2A-2[+]; -1[ζ]-2A-1[+]-1[ζ]-A-1 [+]; -2[+]-A-1[+]-A; -2[+]-1[ζ]-1[+]-A; -1[+]-1[ζ]-A-1[+]-A; -1 [+]-2A-1[+]-1[ζ]-A-1 [+]; and -1[ζ]-A-1[ζ]-A-1[+]; and
[linker] is selected from: -Gn- ; —Sn—; -(GnSn)n- ; -(GnSn)nGn- ; -(GnSn)nSn—; -(GnSn)nGn(GnSn)n-; and -(GnSn)nSn(GnSn)n- ;
wherein:
[Φ] is an amino acid which is: Leu, Phe, Trp, Ile, Met, Tyr, or Val, preferably Leu, Phe, Trp, or Ile;
[+] is an amino acid which is: Lys or Arg;
[ζ] is an amino acid which is: Gln, Asn, Thr, or Ser;
A is the amino acid Ala;
G is the amino acid Gly;
S is the amino acid Ser; and
n is an integer from 1 to 20, 1 to 19, 1 to 18, 1 to 17, 1 to 16, 1 to 15, 1 to 14, 1 to 13, 1 to 12, 1 to 11, 1 to 10,1 to 9,1 to 8,1 to 7,1 to 6,1 to 5,1 to 1 to 4, or 1 to 3.

13. The composition of any one of claims 1 to 12, wherein the shuttle agent comprises or consists of:
(i) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370, or 379;
(ii) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370 or 379 by no more than 1, 2, 3, 4, 5, 6, 7, 8, 9, or 10 amino acids (e.g., excluding any linker domains);
(iii) an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370 or 379 (e.g., calculated excluding any linker domains);
(iv) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, 370 or 379 by only conservative amino acid substitutions (e.g., by no more than no more than 1, 2, 3, 4, 5, 6, 7, 8, 9, or 10 conservative amino acid substitutions, preferably excluding any linker domains), wherein each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and their amides: D, E, N, and Q; or
(v) any combination of (i) to (iv).

14. The composition of any one of claims 1 to 13, wherein the shuttle agent comprises or consists of:
(a) a fragment of a parent shuttle agent as defined in any one of claims 7 to 13, wherein the fragment retains cargo transduction activity and comprises an amphipathic alpha-helical motif having both a positively-charged hydrophilic outer face and a hydrophobic outer face, or
(b) a variant of a parent shuttle agent as defined in any one of claims 7 to 13, wherein the variant retains cargo transduction activity and differs (or differs only) from the parent shuttle agent by having a reduced C-terminal positive charge density relative to the parent shuttle agent (e.g., by substituting one or more cationic residues, such as K/R, with non-cationic residues, preferably non-cationic hydrophilic residues; and/or by engineering hydrophobic residues (e.g., A, V, L, I, F, or W) between two proximal cationic residues;
wherein the fragment or variant has increased resistance to inhibition by the nucleoprotein cargo and/or to the presence of DNA and/or RNA, and/or has increased transduction activity for the nucleoprotein cargo.

15. The composition of claim 14, wherein the fragment or variant comprises or consists of a C-terminal truncation of the parent shuttle agent.

16. The composition of claim 14 or 15, wherein the fragment or variant comprises an amphipathic alpha-helical motif having both a positively-charged hydrophilic outer face and a hydrophobic outer face, which is flanked by or at least by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or non-cationic hydrophilic residues, such that the fragment or variant retains cargo transduction activity and/or has increased resistance to inhibition by the nucleoprotein cargo.

17. The composition of any one of claims 1 to 16, wherein shuttle agent comprises or consists of a peptide less than 20 amino acids in length.

18. The composition of any one of claims 1 to 17, wherein the shuttle agent comprises a helical region comprising an amphipathic helix harboring:
a cluster of hydrophobic amino acid residues on one side of the helix defining a hydrophobic angle of 140° to 280° in Schiffer-Edmundson's wheel representation, and
a cluster of positively charged residues on the other side of the helix defining a positively charged angle of 40° to 160° in Schiffer-Edmundson's wheel representation.

19. The composition of claim 18, wherein the hydrophobic angle is 160° to 260° in Schiffer-Edmundson's wheel representation.

20. The composition of claim 18, wherein the hydrophobic angle is 180° to 240° in Schiffer-Edmundson's wheel representation.

21. The composition of any one of claims 18 to 20, wherein the positively charged angle is 40° to 140° in Schiffer-Edmundson's wheel representation.

22. The composition of any one of claims 18 to 20, wherein the positively charged angle is 60° to 140° in Schiffer-Edmundson's wheel representation.

23. The composition of any one of claims 18 to 20, wherein the positively charged angle is 60° to 120° in Schiffer-Edmundson's wheel representation.

24. The composition of any one of claims 18 to 23, wherein at least 20%, 30%, 40%, or 50% of the residues in the hydrophobic cluster are hydrophobic residues.

25. The composition of claim 24, wherein the hydrophobic residues are selected from the group consisting of phenylalanine, isoleucine, tryptophan, leucine, valine, methionine, tyrosine, cysteine, glycine, and alanine.

26. The composition of claim 24, wherein the hydrophobic residues are selected from the group consisting of phenylalanine, isoleucine, tryptophan, and leucine.

27. The composition of any one of claims 18 to 26, wherein at least 20%, 30%, 40%, or 50% of the residues in the positively charged cluster are positively charged residues.

28. The composition of claim 27, wherein the positively charged residues are selected from the group consisting of lysine, arginine, and histidine.

29. The composition of claim 28, wherein the positively charged residues are selected from the group consisting of lysine and arginine.

30. The composition of any one of 18 to 29, wherein the synthetic peptide shuttle agent is at least 8, 9, 10, 11, 12, 13, 14, 15, 16, 17, 18, or 19 amino acids in length.

31. The composition of any one of claims 1 to 30, wherein the synthetic peptide shuttle agent has a hydrophobic moment (μH) of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5.

32. The composition of any one of claims 1 to 31, wherein the shuttle agent comprises or consists of a variant of the synthetic peptide shuttle agent, the variant being identical to the synthetic peptide shuttle agent as defined in any one of claims 1 or 7 to 31, except having at least one amino acid being replaced with a corresponding synthetic amino acid having a side chain of similar physiochemical properties (e.g., structure, hydrophobicity, or charge) as the amino acid being replaced, wherein the variant increases cytosolic/nuclear delivery of said cargo in eukaryotic cells as compared to in the absence of the synthetic peptide shuttle agent, preferably wherein the synthetic amino acid replacement:
(a) replaces a basic amino acids with any one of: α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine , β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β, β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid; and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.

33. The composition of any one of claims 1 to 32, wherein the shuttle agent:
does not comprise a cell penetrating domain (CPD), a cell-penetrating peptide (CPP), or a protein transduction domain (PTD); or
does not comprise a CPD fused to an endosome leakage domain (ELD).

34. The composition of any one of claims 1 to 32, wherein the shuttle agent comprises an endosome leakage domain (ELD) and/or a cell penetrating domain (CPD).

35. The composition of any one of claim 33 or 34, wherein:
(i) said ELD is or is from: an endosomolytic peptide; an antimicrobial peptide (AMP); a linear cationic alpha-helical antimicrobial peptide; a Cecropin-A/Melittin hybrid (CM) peptide; pH-dependent membrane active peptide (PAMP); a peptide amphiphile; a peptide derived from the N terminus of the HA2 subunit of influenza hemagglutinin (HA); CM18; Diphtheria toxin T domain (DT); GALA; PEA; INF-7; LAH4; HGP; HSWYG; HA2; EB1; VSVG; Pseudomonas toxin; melittin; KALA; JST-1; C(LLKK)₃C; G(LLKK)₃G; or any combination thereof;
(ii) said CPD is or is from: a cell-penetrating peptide or the protein transduction domain from a cell-penetrating peptide; TAT; PTD4; Penetratin; pVEC; M918; Pep-1; Pep-2; Xentry; arginine stretch; transportan; SynB1; SynB3; or any combination thereof; or
(iii) both (i) and (ii).

36. The composition of any one of claims 1 to 35, wherein the shuttle agent is a cyclic peptide and/or comprises one or more D-amino acids.

37. The composition of any one of claims 1 to 36, wherein the shuttle agent increases the transduction efficiency and/or total amount of nucleoprotein cargo delivered intracellularly in the eukaryotic cells by at least 3, 3.5, 4, 4.5, 5, 5.5, 6, 6.5, 7, 7.5, 8, 8.5, 9, 9.5, or 10-fold over a corresponding negative control lacking said shuttle agent.

38. The composition of any one of claims 1 to 37, wherein the shuttle agent further comprises a chemical modification to one or more amino acids, wherein the chemical modification does not destroy the transduction activity of the synthetic peptide shuttle agent.

39. The composition of claim 38, wherein the chemical modification is at the N and/or C terminus of the shuttle agent.

40. The composition of claim 38 or 39, wherein the chemical modification is the addition of an acetyl group (e.g., an N-terminal acetyl group), a cysteamide group (e.g., a C-terminal cysteamide group), or a fatty acid (e.g., C4-C16 fatty acid, preferably N-terminal).

41. The composition of any one of claims 1 to 40, wherein the concentration of the nucleoprotein cargo and/or the synthetic peptide shuttle agent in the composition is at least 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μM.

42. The composition as defined in any one of claims 1 to 41:
(a) for use in increasing the transduction efficiency of the nucleoprotein cargo to the cytosolic/nuclear compartment of eukaryotic cells;
(b) for use in genome editing, base editing, or prime editing in eukaryotic cells;
(c) for use in modulating gene expression in the eukaryotic cells;
(d) for use in therapy, wherein the nucleoprotein cargo binds to a therapeutic target in the eukaryotic cells;
(e) for use in delivering a non-therapeutic nucleoprotein cargo as a diagnostic agent;
(f) for use in the manufacture of a medicament or diagnostic agent;
(g) for use in treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis); or
(h) any combination of (a) to (g).

43. The composition of any one of claims 1 to 41, or the composition for use of claim 42, wherein the eukaryotic cells are animal cells, mammalian cells, human cells, stem cells, primary cells, immune cells, T cells, NK cells, dendritic cells, epithelial cells, skin cells, gastrointestinal cells, lung cells, or ocular cells.

44. A method for the use as defined in claim 42, the method comprising:
(a) providing a nucleoprotein cargo for intracellular delivery in a population of eukaryotic cells;
(b) providing a synthetic peptide shuttle agent that is independent from, or is not covalently linked to, said nucleoprotein cargo;
(c) contacting the eukaryotic cells with the nucleoprotein cargo in the presence of the synthetic peptide shuttle agent at a concentration sufficient to increase the transduction efficiency and/or cytosolic/nuclear delivery of the nucleoprotein cargo, as compared to in the absence of said synthetic peptide shuttle agent,
wherein the nucleoprotein cargo binds to an intracellular target, thereby effecting said use.

45. The method of claim 44, which is an in vitro method (e.g., for a therapeutic and/or diagnostic purpose).

46. The method of claim 44, which is an in vivo method (e.g., for therapeutic and/or diagnostic purpose).

47. The method of any one of claims 44 to 46, wherein:
(i) the nucleoprotein cargo is as defined in any one of claims 1 to 6;
(ii) the synthetic peptide shuttle agent is as defined in any one of claims 1 or 7 to 23;
(iii) the eukaryotic cells are contacted a concentration of the cargo and/or the synthetic peptide shuttle agent as defined in claim 24;
(iv) the method is for a use as defined in claim 25;
(v) the eukaryotic cells are as defined in claim 26; or
(vi) any combination of (i) to (v).

48. A method for cargo transduction, the method comprising contacting target eukaryotic cells with said cargo and a concentration of a synthetic peptide shuttle agent sufficient to increase the transduction efficiency of said cargo, as compared to in the absence of said synthetic peptide shuttle agent, wherein said synthetic peptide shuttle agent is a peptide less than 20 amino acids in length, and wherein the shuttle agent and cargo are not covalently bound at the time of transduction across the plasma membrane.

49. The method of claim 48, wherein the synthetic peptide shuttle agent comprises a helical region comprising an amphipathic helix harboring:
a cluster of hydrophobic amino acid residues on one side of the helix defining a hydrophobic angle of 140° to 280° in Schiffer-Edmundson's wheel representation, and
a cluster of positively charged residues on the other side of the helix defining a positively charged angle of 40° to 160° in Schiffer-Edmundson's wheel representation.

50. The method of claim 49, wherein the hydrophobic angle is 160° to 260° in Schiffer-Edmundson's wheel representation.

51. The method of claim 49, wherein the hydrophobic angle is 180° to 240° in Schiffer-Edmundson's wheel representation.

52. The method of any one of claims 49 to 51, wherein the positively charged angle is 40° to 140° in Schiffer-Edmundson's wheel representation.

53. The method of any one of claims 49 to 51, wherein the positively charged angle is 60° to 140° in Schiffer-Edmundson's wheel representation.

54. The method of any one of claims 49 to 51, wherein the positively charged angle is 60° to 120° in Schiffer-Edmundson's wheel representation.

55. The method of any one of claims 49 to 54, wherein at least 20%, 30%, 40%, or 50% of the residues in the hydrophobic cluster are hydrophobic residues.

56. The method of claim 55, wherein the hydrophobic residues are selected from the group consisting of phenylalanine, isoleucine, tryptophan, leucine, valine, methionine, tyrosine, cysteine, glycine, and alanine.

57. The method of claim 55, wherein the hydrophobic residues are selected from the group consisting of phenylalanine, isoleucine, tryptophan, and/or leucine.

58. The method of any one of claims 49 to 57, wherein at least 20%, 30%, 40%, or 50% of the residues in the positively charged cluster are positively charged residues.

59. The method of any one of claims 49 to 58, wherein the positively charged residues are selected from the group consisting of lysine, arginine, and histidine.

60. The method of any one of claims 49 to 58, wherein the positively charged residues are selected from the group consisting of lysine and arginine.

61. The method of any one of claims 49 to 60, wherein the synthetic peptide shuttle agent is at least 8, 9, 10, 11, 12, 13, 14, 15, 16, 17, 18, or 19 amino acids in length.

62. The method of any one of claims 49 to 61, wherein the synthetic peptide shuttle agent has a hydrophobic moment (μH) of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5.

63. The method of any one of claims 49 to 62, wherein the cargo is a polypeptide, peptide, nucleoprotein (e.g., as defined in any one of claims 1 to 6), small molecule, oligonucleotide, or oligonucleotide analog (e.g., non-anionic oligonucleotide analog).

64. A synthetic peptide which is the shuttle agent as defined in any one of claims 1 to 63.

65. The synthetic peptide of claim 64 for use in therapy and/or in cargo transduction (e.g., polypeptide, peptide, nucleoprotein (e.g., as defined in any one of claims 1 to 6), small molecule, oligonucleotide, or oligonucleotide analog (e.g., non-anionic oligonucleotide analog) in eukaryotic cells, wherein the shuttle agent is used at concentration sufficient to increase the transduction efficiency of said cargo, as compared to in the absence of said synthetic peptide shuttle agent, and wherein the synthetic peptide shuttle agent and cargo are not covalently bound.

66. Use of the synthetic peptide shuttle agent as defined in any one of claims 1 to 63 for the manufacture of a medicament (e.g., for treating a disease as defined in claim 42).

67. A composition comprising the synthetic peptide shuttle agent of claim 64 and a suitable excipient.

68. A synthetic peptide shuttle agent for use in, or suitable for use in, the delivery of non-anionic cargoes across mucus-producing membranes (e.g., airway epithelium), the synthetic peptide shuttle agent comprising or consisting essentially of a central core amphipathic alpha helical region having shuttle agent activity, flanked N- and C-terminally by flexible linker domains, wherein one or both of the flexible linker domains comprises or consists essentially of a sufficient number of non-cationic hydrophilic residues such that cargo transduction activity across mucus-producing membranes of the synthetic peptide shuttle agent is increased relative to that of the central core amphipathic alpha helical region lacking the flexible linker domains.

69. The synthetic peptide shuttle agent of claim 68, wherein the central core amphipathic alpha helical region:
(a) is an endosomolytic peptide;
(b) is at least 8, 9, 10, 11, 12, 13, 14, 15, 16, 17, 18, or 19 amino acids in length;
(c) is a fragment of a parent shuttle agent as defined in claim 14(a) or 15;
(d) is an amphipathic helix as defined in any one of claims 18 to 29 or 49 to 60;
(e) has a hydrophobic moment (μH) of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5; or
(f) any combination of (a) to (e).

70. The synthetic peptide shuttle agent of claim 68 or 69, wherein the non-cationic hydrophilic residues comprise or consist essentially of glycine, serine, aspartate, glutamate, histidine, tyrosine, threonine, cysteine, asparagine, glutamine, or any combination thereof.

71. The synthetic peptide shuttle agent of any one of claims 68 to 70, wherein the flexible linker domain is as defined in claim 12.

72. The synthetic peptide shuttle agent of any one of claims 68 to 71 for the use as defined in claim 42 or 43.


SEQUENCE LISTING (381 sequences)

SEQ ID NO: 1 - protein, 35 aa, Artificial Sequence
  CM18-Penetratin-cys
KWKLFKKIGAVLKVLTTGRQIKIWFQNRRMKWKKC

SEQ ID NO: 2 - protein, 41 aa, Artificial Sequence
  TAT-KALA
YGRKKRRQRRRWEAKLAKALAKALAKHLAKALAKALKACEA

SEQ ID NO: 3 - protein, 35 aa, Artificial Sequence
  His-CM18-PTD4
HHHHHHKWKLFKKIGAVLKVLTTGYARAAARQARA

SEQ ID NO: 4 - protein, 43 aa, Artificial Sequence
  His-LAH4-PTD4
HHHHHHKKALLALALHHLAHLALHLALALKKAYARAAARQARA

SEQ ID NO: 5 - protein, 41 aa, Artificial Sequence
  PTD4-KALA
YARAAARQARAWEAKLAKALAKALAKHLAKALAKALKACEA

SEQ ID NO: 6 - protein, 34 aa, Artificial Sequence
  EB1-PTD4
LIRLWSHLIHIWFQNRRLKWKKKYARAAARQARA

SEQ ID NO: 7 - protein, 41 aa, Artificial Sequence
  His-CM18-PTD4-6Cys
HHHHHHKWKLFKKIGAVLKVLTTGYARAAARQARACCCCCC

SEQ ID NO: 8 - protein, 29 aa, Artificial Sequence
  CM18-PTD4
KWKLFKKIGAVLKVLTTGYARAAARQARA

SEQ ID NO: 9 - protein, 35 aa, Artificial Sequence
  CM18-PTD4-6His
KWKLFKKIGAVLKVLTTGYARAAARQARAHHHHHH

SEQ ID NO: 10 - protein, 41 aa, Artificial Sequence
  His-CM18-PTD4-His
HHHHHHKWKLFKKIGAVLKVLTTGYARAAARQARAHHHHHH

SEQ ID NO: 11 - protein, 30 aa, Artificial Sequence
  TAT-CM18
YGRKKRRQRRRCKWKLFKKIGAVLKVLTTG

SEQ ID NO: 12 - protein, 37 aa, Artificial Sequence
  FSD5
HHHHHHLLKLWSRLLKLWTQGRRLKAKRAKAHHHHHH

SEQ ID NO: 13 - protein, 34 aa, Artificial Sequence
  FSD10
KWKLARAFARAIKKLGGSGGGSYARALRRQARTG

SEQ ID NO: 14 - protein, 27 aa, Artificial Sequence
  FSD12
KWKLARAFARAIKKLYARALRRQARTG

SEQ ID NO: 15 - protein, 32 aa, Artificial Sequence
  FSD18
LLKLWSRLLKLWTQGGSGGGSGRRLKAKRAKA

SEQ ID NO: 16 - protein, 42 aa, Artificial Sequence
  FSD19
HHHHHHLLKLWSRLLKTWTQGRRLKAKSAQASTRQAHHHHHH

SEQ ID NO: 17 - protein, 31 aa, Artificial Sequence
  FSD21
HHHHHHFLKIWSRLIKIWTQGRRKGAQAAFR

SEQ ID NO: 18 - protein, 37 aa, Artificial Sequence
  FSD23
HHHHHHLLKLWSRLLKEWTQGRRLEAKRAEAHHHHHH

SEQ ID NO: 19 - protein, 31 aa, Artificial Sequence
  FSD120
HHHHHHFLKIWSRLIKIWTQGLRKGAQAAKR

SEQ ID NO: 20 - protein, 30 aa, Artificial Sequence
  FSD127
HHHHHHLLKIWSRLIKGWTQGWRTIAQALG

SEQ ID NO: 21 - protein, 31 aa, Artificial Sequence
  FSD129
FLKIWSRLIKIWTQGGSGGGSRRKGAQAAFR

SEQ ID NO: 22 - protein, 37 aa, Artificial Sequence
  FSD131
HHHHHHFLKIWSRLIKIWTQGRRKGAQAAFRHHHHHH

SEQ ID NO: 23 - protein, 30 aa, Artificial Sequence
  FSD134
LIRKWIHLIHSWFQNLRRLYARAAARQARA

SEQ ID NO: 24 - protein, 32 aa, Artificial Sequence
  FSD146
LLKLWSRLLKLWTQGGSPPPSGRRLKAKRAKA

SEQ ID NO: 25 - protein, 32 aa, Artificial Sequence
  FSD155
LLKLWSRLLKLWTQGGSGEGSGRRLKAKRAKA

SEQ ID NO: 26 - protein, 23 aa, Artificial Sequence
  FSD156
WIRLFTKLWRIFQQGKRIKAKRA

SEQ ID NO: 27 - protein, 29 aa, Artificial Sequence
  FSD157
WIRLFTKLWRIFQQGGSGGGSKRIKAKRA

SEQ ID NO: 28 - protein, 29 aa, Artificial Sequence
  FSD159
WIRLFTKLWRIFRQGGSGGGSKRIKAKAA

SEQ ID NO: 29 - protein, 25 aa, Artificial Sequence
  FSD162
ILKLWSRLIKIWTQGRRKKAQAAKR

SEQ ID NO: 30 - protein, 25 aa, Artificial Sequence
  FSD168
LLKIWSRLIKIWTQGRRLGARAQAR

SEQ ID NO: 31 - protein, 27 aa, Artificial Sequence
  FSD173
LLKIWSRLIKIWTQGRRLGARAARQAR

SEQ ID NO: 32 - protein, 33 aa, Artificial Sequence
  FSD174
LLKIWSRLIKIWTQGRRLGGSGGGSARAARQAR

SEQ ID NO: 33 - protein, 32 aa, Artificial Sequence
  FSD194
KWKLLKLWSRLLKLWGGSGGGSRRLKAKRAKA

SEQ ID NO: 34 - protein, 23 aa, Artificial Sequence
  FSD220
WARAFAKAWRIFQQGKRIKAKRA

SEQ ID NO: 35 - protein, 30 aa, Artificial Sequence
  FSD250
KWKLLKLWSRLLKLWGGSGGGSARAARQAR

SEQ ID NO: 36 - protein, 30 aa, Artificial Sequence; identical to SEQ ID NO: 35
  FSD250D
  MISC_FEATURE (1)..(30): All D-amino acids

SEQ ID NO: 37 - protein, 32 aa, Artificial Sequence
  FSD253
KWKLLKLWSRLLKLWGGSRGGRGSARAARQAR

SEQ ID NO: 38 - protein, 29 aa, Artificial Sequence
  FSD258
WKLLKLWSRLLKLWGGSGGGSARAARQAR

SEQ ID NO: 39 - protein, 30 aa, Artificial Sequence
  FSD262
KWKLLRLWSRLLRLWGGSGGGSARAARQAR

SEQ ID NO: 40 - protein, 31 aa, Artificial Sequence
  FSD263
KWKLLKLWSRLLKLWGGSGGGSARAAARQAR

SEQ ID NO: 41 - protein, 31 aa, Artificial Sequence
  FSD264
KWKLLKLWSRLLKLWGGSGGGSARAAARAAR

SEQ ID NO: 42 - protein, 32 aa, Artificial Sequence
  FSD265
KWKLLKLWSRLLKLWGGSGGGSQARAAARQAR

SEQ ID NO: 43 - protein, 32 aa, Artificial Sequence
  FSD268
KWKLLKLWSRLLKLWGGSGGGSQARAQARQAR

SEQ ID NO: 44 - protein, 33 aa, Artificial Sequence
  FSD286
KWKLLRALARLLKLAGGSGGGSQRRLGARAQAR

SEQ ID NO: 45 - protein, 31 aa, Artificial Sequence
  FSD271
KWKLLKLWSRLLKLWRGGSGGGSARAARQAR

SEQ ID NO: 46 - protein, 30 aa, Artificial Sequence
  FSD272
KWKLAKLWSRLLKLWGGSGGGSARAARQAR

SEQ ID NO: 47 - protein, 30 aa, Artificial Sequence
  FSD273
KWKLLRAWSRLLKLWGGSGGGSARAARQAR

SEQ ID NO: 48 - protein, 30 aa, Artificial Sequence
  FSD276
KWKLLKLWSRLLRAWGGSGGGSARAARQAR

SEQ ID NO: 49 - protein, 32 aa, Artificial Sequence; identical to SEQ ID NO: 43
  FSD268 Cyclic Amide
  MISC_FEATURE (1)..(32): Cyclic peptide covalent link between K1 and R32

SEQ ID NO: 50 - protein, 34 aa, Artificial Sequence
  FSD268 Disulfide
  MISC_FEATURE (1)..(32): Cyclic peptide disulfiude bond between C1 and C34
CKWKLLKLWSRLLKLWGGSGGGSQARAQARQARC

SEQ ID NO: 51 - protein, 34 aa, Artificial Sequence; identical to SEQ ID NO: 13
  FSD10 Scarmble

SEQ ID NO: 52 - protein, 32 aa, Artificial Sequence; identical to SEQ ID NO: 43
  FSD268 Scramble

SEQ ID NO: 53 - protein, 33 aa, Artificial Sequence
  FSD174 Scramble
LGRSGRIKIGGWSALASRARQARGLKIWTQGRL

SEQ ID NO: 54 - protein, 36 aa, Artificial Sequence
  FSN3
HHHHHHQFLCFWLNKMGKHNTVWHGRHLKCHKRGKG

SEQ ID NO: 55 - protein, 35 aa, Artificial Sequence
  FSN4
HHHHHHLLYLWRRLLKFWCAGRRVYAKCAKAYGCF

SEQ ID NO: 56 - protein, 31 aa, Artificial Sequence
  FSN7
LIKLWSRFIKFWTQGRRIKAKLARAGQSWFG

SEQ ID NO: 57 - protein, 19 aa, Artificial Sequence
  FSN8
HHHHHHFRKLWLAIVRAKK

SEQ ID NO: 58 - protein, 31 aa, Artificial Sequence
  FSD117
HHHHHHFLKFWSRLFKFWTQGRRKGAQAAFR

SEQ ID NO: 59 - protein, 31 aa, Artificial Sequence
  FSD118
HHHHHHILKIWSRLIKIWTQGRRKGAQAAIR

SEQ ID NO: 60 - protein, 32 aa, Artificial Sequence
  FSD119
HHHHHHFLKIWSRALIKIWTQGLRKGAQAAKR

SEQ ID NO: 61 - protein, 31 aa, Artificial Sequence
  FSD121
HHHHHHVLKIWSRLIKIWTQGRRKGAQAAVR

SEQ ID NO: 62 - protein, 31 aa, Artificial Sequence
  FSD122
HHHHHHFLKVWSRLVKVWTQGRRKGAQAAFR

SEQ ID NO: 63 - protein, 31 aa, Artificial Sequence
  FSD123
HHHHHHVLKVWSRLVKVWTQGRRKGAQAAVR

SEQ ID NO: 64 - protein, 31 aa, Artificial Sequence
  FSD124
HHHHHHFLKIWQRLIKIWQQGRRKGAQAAFR

SEQ ID NO: 65 - protein, 31 aa, Artificial Sequence
  FSD125
HHHHHHFLKIWNRLIKIWNNGRRKGANAAFR

SEQ ID NO: 66 - protein, 30 aa, Artificial Sequence
  FSD126
HHHHHHFLKIWSRLIKIWTQGWRTGAQAGF

SEQ ID NO: 67 - protein, 30 aa, Artificial Sequence; identical to SEQ ID NO: 20
  FSD127

SEQ ID NO: 68 - protein, 31 aa, Artificial Sequence
  FSD128
HHHHHHFLKIWSRLIKIWPQPRRKGAQAAFR

SEQ ID NO: 69 - protein, 31 aa, Artificial Sequence
  FSD130
LIKIWTQFLKIWSRGGSGGGSRRLGARAQAR

SEQ ID NO: 70 - protein, 31 aa, Artificial Sequence
  FSD132
HHHHHHRFAAQAGKRRGQTWIKILRSWIKLF

SEQ ID NO: 71 - protein, 31 aa, Artificial Sequence
  FSD133
HHHHFLHHSWIKKILRTWIRRGQQAGKFAAR

SEQ ID NO: 72 - protein, 29 aa, Artificial Sequence
  FSD135
LIRKWIHLIHSWFQNLRRLQARAQARQAR

SEQ ID NO: 73 - protein, 29 aa, Artificial Sequence
  FSD137
LLRKWSHLLHIWGGSGGGSQARAQARQAR

SEQ ID NO: 74 - protein, 33 aa, Artificial Sequence
  FSD138
KWKLARAFARAIKIFGGSGGGSQRRLKAKRAKA

SEQ ID NO: 75 - protein, 40 aa, Artificial Sequence
  FSD139
HHHHHHLIRLWSHLIHIWFQNRRLKWKKKYARAAARQARA

SEQ ID NO: 76 - protein, 46 aa, Artificial Sequence
  FSD140
HHHHHHLIRLWSHLIHIWFQNRRLKWKKKYARAAARQARAHHHHHH

SEQ ID NO: 77 - protein, 41 aa, Artificial Sequence
  FSD141
LIRLWSHLIHIWFQNRRLKWKKKGGSGGGSYARAAARQARA

SEQ ID NO: 78 - protein, 25 aa, Artificial Sequence
  FSD142
FLKIWSHLIHIWTQGRRLKAKRAKA

SEQ ID NO: 79 - protein, 22 aa, Artificial Sequence
  FSD143
LIRKWIHLIHSWFQGRRLGARA

SEQ ID NO: 80 - protein, 49 aa, Artificial Sequence
  FSD144
HHHHHHKKALLAHALHLLALLALHLAHALKKAYGRKKRRQRRRHHHHHH

SEQ ID NO: 81 - protein, 52 aa, Artificial Sequence
  FSD145
HHHHHHKKHLLAHALHLLALLALHLAHALAHLKKAYGRKKRRQRRRHHHHHH

SEQ ID NO: 82 - protein, 31 aa, Artificial Sequence
  FSD147
LLKLWTQLLKLWSRGGSGGGSRRLKAKRAKA

SEQ ID NO: 83 - protein, 28 aa, Artificial Sequence
  FSD148
HHHHHHMVTVLFRRLRIRRACGPPRVRV

SEQ ID NO: 84 - protein, 28 aa, Artificial Sequence
  FSD149
HHHHHHMVRVLTRFLRIGARCRRPPVVR

SEQ ID NO: 85 - protein, 30 aa, Artificial Sequence
  FSD150
HHHHHHWITWLFKRLKIRRAAGQSKFRIAG

SEQ ID NO: 86 - protein, 30 aa, Artificial Sequence
  FSD151
HHHHHHWITWLRKILKRFRKAAQSGFRIAG

SEQ ID NO: 87 - protein, 30 aa, Artificial Sequence
  FSD152
HHHHHHWITWLRKILKRFGKAAQSGFRIAR

SEQ ID NO: 88 - protein, 30 aa, Artificial Sequence
  FSD153
HHHHHHWITWLRKILKRLGGAAQSIITGGQ

SEQ ID NO: 89 - protein, 36 aa, Artificial Sequence
  FSD154
HHHHHHWITWLFKRLKIRRAAGGSGGGSQSKFRIAG

SEQ ID NO: 90 - protein, 23 aa, Artificial Sequence
  FSD158
WIRLFTKLWRIFRQGKRIKAKAA

SEQ ID NO: 91 - protein, 25 aa, Artificial Sequence
  FSD160
ILKLWSRLIKIWTQGRRLGAQAALR

SEQ ID NO: 92 - protein, 31 aa, Artificial Sequence
  FSD161
ILKLWSRLIKIWTQGGSGGGSRRLGAQAALR

SEQ ID NO: 93 - protein, 31 aa, Artificial Sequence
  FSD163
ILKLWSRLIKIWTQGGSGGGSRRKKAQAAKR

SEQ ID NO: 94 - protein, 25 aa, Artificial Sequence
  FSD164
LLKIWSRLIKIWTQGRRLGARAARA

SEQ ID NO: 95 - protein, 31 aa, Artificial Sequence
  FSD165
LLKIWSRLIKIWTQGGSGGGSRRKKARAARA

SEQ ID NO: 96 - protein, 26 aa, Artificial Sequence
  FSD166
LLKLWSRLIKIWTKGRRKKARAAQAR

SEQ ID NO: 97 - protein, 32 aa, Artificial Sequence
  FSD167
LLKLWSRLIKIWTKGGSGGGSRRKKARAAQAR

SEQ ID NO: 98 - protein, 31 aa, Artificial Sequence
  FSD169
LLKIWSRLIKIWTQGGSGGGSRRLGARAQAR

SEQ ID NO: 99 - protein, 25 aa, Artificial Sequence
  FSD170
LIKIWTQLLKIWSRGRRLGARAQAR

SEQ ID NO: 100 - protein, 25 aa, Artificial Sequence
  FSD171
  MISC_FEATURE (1)..(1): Acetyl
  MISC_FEATURE (25)..(25): Amide
  MISC_FEATURE (25)..(25): Cysteamide
LLKIWSRLIKIWTQGRRLGARAQAR

SEQ ID NO: 101 - protein, 31 aa, Artificial Sequence
  FSD172
LLKIWSRLIKIWTQGRRLGGSGGGSARAQAR

SEQ ID NO: 102 - protein, 29 aa, Artificial Sequence
  FSD175
LLKIWSRLIKIWTQGRRLGGSARAARQAR

SEQ ID NO: 103 - protein, 36 aa, Artificial Sequence
  FSD176
LLKIWSRLIKIWTQGRRLGGSGGGSGGSARAARQAR

SEQ ID NO: 104 - protein, 25 aa, Artificial Sequence
  FSD177
KLKIWSRLIRKWTKGLRLGAQAQAR

SEQ ID NO: 105 - protein, 31 aa, Artificial Sequence
  FSD178
KLKIWSRLIRKWTKGGSGGGSLRLGAQAQAR

SEQ ID NO: 106 - protein, 28 aa, Artificial Sequence
  FSD179
LLKIWSRLIKIWTQGRGRESRKPRKSRQ

SEQ ID NO: 107 - protein, 34 aa, Artificial Sequence
  FSD180
LLKIWSRLIKIWTQGGSGGGSRGRESRKPRKSRQ

SEQ ID NO: 108 - protein, 28 aa, Artificial Sequence
  FSD181
LLKIWSRLIKIWTQGLGLLVLRVRAGKR

SEQ ID NO: 109 - protein, 34 aa, Artificial Sequence
  FSD182
LLKIWSRLIKIWTQGGSGGGSLGLLVLRVRAGKR

SEQ ID NO: 110 - protein, 22 aa, Artificial Sequence
  FSD183
LLKIWSRLIKIWTQGRRLGARA

SEQ ID NO: 111 - protein, 24 aa, Artificial Sequence
  FSD184
LLKIWSRLIKIWTQGRRLGARAAR

SEQ ID NO: 112 - protein, 25 aa, Artificial Sequence
  FSD185
LLKIWSRLIKIWTQGRRLGARAARQ

SEQ ID NO: 113 - protein, 27 aa, Artificial Sequence
  FSD186
LLKIWSRLIKIWTQGRGLEARAPRKAR

SEQ ID NO: 114 - protein, 28 aa, Artificial Sequence
  FSD187
LLKIWSRLIKIWTQGRRLGARKPRKSRQ

SEQ ID NO: 115 - protein, 27 aa, Artificial Sequence
  FSD188
LLKIWSRLIKIWTQGRGRESRAARQAR

SEQ ID NO: 116 - protein, 27 aa, Artificial Sequence
  FSD189
LLKIWSRLIKIWTQGRRLGRAQRAQRA

SEQ ID NO: 117 - protein, 23 aa, Artificial Sequence
  FSD190
LLKIWSRLIKIWTQGRAQRAQRA

SEQ ID NO: 118 - protein, 32 aa, Artificial Sequence
  FSD191
HHHHHHLLKIWSRLIKIWTQGTRSKRAGLQFP

SEQ ID NO: 119 - protein, 31 aa, Artificial Sequence
  FSD192
HHHHHHLLKIWSRLIKIWTQGVGRVHRLLRK

SEQ ID NO: 120 - protein, 33 aa, Artificial Sequence
  FSD193
KWKLLKIWSRLIKIWRRLGGSGGGSARAARQAR

SEQ ID NO: 121 - protein, 25 aa, Artificial Sequence
  FSD195
LLKIWSRLIKIWTQGRRLKARAQAR

SEQ ID NO: 122 - protein, 25 aa, Artificial Sequence
  FSD196
LLKIWSRLIKIWTQGRRLGARAAAR

SEQ ID NO: 123 - protein, 25 aa, Artificial Sequence
  FSD197
LLKIWSRLIKIWTQGRRLKARAAAR

SEQ ID NO: 124 - protein, 27 aa, Artificial Sequence
  FSD198
WSRLIKIWTQGGSGGGSRRKGAQAAFR

SEQ ID NO: 125 - protein, 23 aa, Artificial Sequence
  FSD199
WSRLITKIWRIFTQGRRLGARAA

SEQ ID NO: 126 - protein, 23 aa, Artificial Sequence
  FSD200
WSRLITKIWRIFTQGRRLKARAA

SEQ ID NO: 127 - protein, 19 aa, Artificial Sequence
  FSD201
WSRLIKLWTQGRRLKARAA

SEQ ID NO: 128 - protein, 19 aa, Artificial Sequence
  FSD202
WIRLFKLWQQGKRIKAKRA

SEQ ID NO: 129 - protein, 21 aa, Artificial Sequence
  FSD203
WSRLIKIWTQGRRLGARAQAR

SEQ ID NO: 130 - protein, 21 aa, Artificial Sequence
  FSD204
LLKIWSRLIKIWTQGRRLGAR

SEQ ID NO: 131 - protein, 17 aa, Artificial Sequence
  FSD205
WSRLIKIWTQGRRLGAR

SEQ ID NO: 132 - protein, 21 aa, Artificial Sequence
  FSD206
KIWSRLIKIWTQGRRLGARAQ

SEQ ID NO: 133 - protein, 25 aa, Artificial Sequence
  FSD207
LAKAWARAIKIWTQGRRLGARAQAR

SEQ ID NO: 134 - protein, 32 aa, Artificial Sequence
  FSD208
KWKLARAFARAIKKLGGSGGGSRRLGARAQAR

SEQ ID NO: 135 - protein, 33 aa, Artificial Sequence
  FSD209
LLKIWSRLIKIWTQGGSGGGSYARALRRQARTG

SEQ ID NO: 136 - protein, 32 aa, Artificial Sequence
  FSD210
KWKLARAFARAIKKLGGSGGGSRRLKAKRAKA

SEQ ID NO: 137 - protein, 34 aa, Artificial Sequence
  FSD211
KWKLLKLWSRLLKLWGGSGGGSYARALRRQARTG

SEQ ID NO: 138 - protein, 25 aa, Artificial Sequence
  FSD212
WSRLLKLWGGSGGGSRRLKAKRAKA

SEQ ID NO: 139 - protein, 25 aa, Artificial Sequence
  FSD213
LLKLWSRLLKLWGGSGGGSRRLKAK

SEQ ID NO: 140 - protein, 21 aa, Artificial Sequence
  FSD214
WSRLLKLWGGSGGGSRRLKAK

SEQ ID NO: 141 - protein, 25 aa, Artificial Sequence
  FSD215
KLWSRLLKLWGGSGGGSRRLKAKRA

SEQ ID NO: 142 - protein, 27 aa, Artificial Sequence
  FSD216
LLKIWSRLIKIWTQGRGRSRKPRKSRQ

SEQ ID NO: 143 - protein, 22 aa, Artificial Sequence
  FSD217
KWKLKLWRLKGGSGGGSRRAKA

SEQ ID NO: 144 - protein, 31 aa, Artificial Sequence
  FSD218
KWKLKLWRLKSRLKLWRLKGGSGGGSRRAKA

SEQ ID NO: 145 - protein, 23 aa, Artificial Sequence
  FSD219
WIRLWTHLWHIWQQGKRIKAKRA

SEQ ID NO: 146 - protein, 24 aa, Artificial Sequence
  FSD221
WKLIRLFTRLIKIWGQRRLKAKRA

SEQ ID NO: 147 - protein, 22 aa, Artificial Sequence
  FSD222
LARAFARAIKIFGQRRLKAKRA

SEQ ID NO: 148 - protein, 28 aa, Artificial Sequence
  FSD223
LARAFARAIKIFQGGSGGGSRRLKAKRA

SEQ ID NO: 149 - protein, 25 aa, Artificial Sequence
  FSD224
KWKLARAFARAIKIFGQRRLKAKRA

SEQ ID NO: 150 - protein, 31 aa, Artificial Sequence
  FSD225
KWKLARAFARAIKIFGGSGGGSQRRLKAKRA

SEQ ID NO: 151 - protein, 27 aa, Artificial Sequence
  FSD226
KWKLARAFARAIKIFGQRRLGARAQAR

SEQ ID NO: 152 - protein, 33 aa, Artificial Sequence
  FSD227
KWKLARAFARAIKIFGGSGGGSQRRLGARAQAR

SEQ ID NO: 153 - protein, 27 aa, Artificial Sequence
  FSD228
KWKLARAFARAIKIFGQRRLKAKRAKA

SEQ ID NO: 154 - protein, 33 aa, Artificial Sequence; identical to SEQ ID NO: 74
  FSD229

SEQ ID NO: 155 - protein, 26 aa, Artificial Sequence
  FSD230
KWKLAKAWARALKLWGRRLGARAQAR

SEQ ID NO: 156 - protein, 33 aa, Artificial Sequence
  FSD231
KWKLARAFARAIKKLGGSGGGSRRLKAKRALKA

SEQ ID NO: 157 - protein, 32 aa, Artificial Sequence
  FSD232
KWKWARAWARAWKKWGGSGGGSRRLGARAQAR

SEQ ID NO: 158 - protein, 32 aa, Artificial Sequence
  FSD233
KLKLARALARALKKLGGSGGGSRRLGARAQAR

SEQ ID NO: 159 - protein, 32 aa, Artificial Sequence
  FSD234
KIKIARAIARAIKKIGGSGGGSRRLGARAQAR

SEQ ID NO: 160 - protein, 32 aa, Artificial Sequence
  FSD235
KFKFARAFARAFKKFGGSGGGSRRLGARAQAR

SEQ ID NO: 161 - protein, 39 aa, Artificial Sequence
  FSD236
KWKLLKLWSRLLKLWSRLLKLWGGSGGGSRRLGARAQAR

SEQ ID NO: 162 - protein, 39 aa, Artificial Sequence
  FSD237
KWKLLKLWTQLLKLWTQLLKLWGGSGGGSRRLGARAQAR

SEQ ID NO: 163 - protein, 32 aa, Artificial Sequence
  FSD238
KWKLLKIWSRLIKIWGGSGGGSQARAQARQAR

SEQ ID NO: 164 - protein, 31 aa, Artificial Sequence
  FSD239
KWKLLKIWTQLIKIWGGSGGGSQARQARQAR

SEQ ID NO: 165 - protein, 33 aa, Artificial Sequence
  FSD240
KWKALLALALHLAHLALHLKKAGRRKGAQAAFR

SEQ ID NO: 166 - protein, 33 aa, Artificial Sequence
  FSD241
KWKLARAFARAIKKLGGSGGGSYARAAARQARA

SEQ ID NO: 167 - protein, 36 aa, Artificial Sequence
  FSD243
LLKIWSRLIKIWTQGRRLGGSGGGSYARAAARQARA

SEQ ID NO: 168 - protein, 34 aa, Artificial Sequence
  FSD244
KWKLAKAWARALKLWGGSGGGSYARAAARKAKRA

SEQ ID NO: 169 - protein, 34 aa, Artificial Sequence
  FSD246
KWKLLKLWSRLLKLWGGSGGGSYARAAARKAKRA

SEQ ID NO: 170 - protein, 37 aa, Artificial Sequence
  FSD247
LLKIWSRLIKIWTQGRRLGGSGGGSYARAAARKAKRA

SEQ ID NO: 171 - protein, 30 aa, Artificial Sequence
  FSD248
KWKLAKAWARALKLWGGSGGGSARAARQAR

SEQ ID NO: 172 - protein, 30 aa, Artificial Sequence
  FSD250 Scramble
RGKLWSLSKLKGWGGARASKAQLARLGLWR

SEQ ID NO: 173 - protein, 30 aa, Artificial Sequence
  FSD250E
KWKLLELWSELLELWGGSGGGSARAARQAR

SEQ ID NO: 174 - protein, 30 aa, Artificial Sequence
  FSD251
KWKLLKLWSRLLKLWGGSGGGSAEAAEQAE

SEQ ID NO: 175 - protein, 32 aa, Artificial Sequence; identical to SEQ ID NO: 37
  FSD254

SEQ ID NO: 176 - protein, 32 aa, Artificial Sequence
  FSD255
KWKLLKLWGGSRLLKLWGGSGGGSARAARQAR

SEQ ID NO: 177 - protein, 33 aa, Artificial Sequence
  FSD256
KWKLLKLGRWSRLGLKLWGGSGGGSARAARQAR

SEQ ID NO: 178 - protein, 32 aa, Artificial Sequence
  FSD257
KWKLLKLWAASRLLKLWGGSGGGSARAARQAR

SEQ ID NO: 179 - protein, 33 aa, Artificial Sequence
  FSD259
KWKLLKLARWSRLALKLWGGSGGGSARAARQAR

SEQ ID NO: 180 - protein, 30 aa, Artificial Sequence
  FSD260
RWRLLRLWSRLLRLWGGSGGGSARAARQAR

SEQ ID NO: 181 - protein, 30 aa, Artificial Sequence
  FSD261
GGSLLKLWSRLLKLWGGSGGGSARAARQAR

SEQ ID NO: 182 - protein, 27 aa, Artificial Sequence
  FSD266
LLKLWSRLLKLWGGSGGGSARAARQAR

SEQ ID NO: 183 - protein, 31 aa, Artificial Sequence
  FSD267
KWKLLKLWSRLLKLWGGSGGGSYARAARYAR

SEQ ID NO: 184 - protein, 32 aa, Artificial Sequence
  FSD269
KWKLLKLWSRLLKLWGGSGGGSYARAYARYAR

SEQ ID NO: 185 - protein, 28 aa, Artificial Sequence
  FSD270
KWKLLKLWSRLLKLWGGSGGGSAAAAEK

SEQ ID NO: 186 - protein, 30 aa, Artificial Sequence
  FSD274
KWKLARAWSRLLKLWGGSGGGSARAARQAR

SEQ ID NO: 187 - protein, 30 aa, Artificial Sequence
  FSD275
KWKLLKLWSRLAKLWGGSGGGSARAARQAR

SEQ ID NO: 188 - protein, 30 aa, Artificial Sequence; identical to SEQ ID NO: 48
  FSD276

SEQ ID NO: 189 - protein, 30 aa, Artificial Sequence
  FSD277
KWKLLKLWSRLARAWGGSGGGSARAARQAR

SEQ ID NO: 190 - protein, 30 aa, Artificial Sequence
  FSD278
KWKLARAWSRLARAWGGSGGGSARAARQAR

SEQ ID NO: 191 - protein, 29 aa, Artificial Sequence
  FSD279
KWKLARALARAWSRGGSGGGSARAARQAR

SEQ ID NO: 192 - protein, 30 aa, Artificial Sequence
  FSD280
KWKLLKLWKRLLKKWGGSGGGSARAARQAR

SEQ ID NO: 193 - protein, 30 aa, Artificial Sequence
  FSD281
KWSLLKLWSALLKLWGGSGGGSARAARQAR

SEQ ID NO: 194 - protein, 30 aa, Artificial Sequence
  FSD282
KWKLWKLLSRLWKLLGGSGGGSARAARQAR

SEQ ID NO: 195 - protein, 30 aa, Artificial Sequence
  FSD283
KWKLARKFKRAIKKFGGSGGGSARAARQAR

SEQ ID NO: 196 - protein, 30 aa, Artificial Sequence
  FSD284
KWALARAFARAIAIFGGSGGGSARAARQAR

SEQ ID NO: 197 - protein, 30 aa, Artificial Sequence
  FSD285
LARAFARAIKIFGGSGGGSQRRLGARAQAR

SEQ ID NO: 198 - protein, 33 aa, Artificial Sequence
  FSD287
KWKLLRALARLLKALGGSGGGSQRRLGARAQAR

SEQ ID NO: 199 - protein, 32 aa, Artificial Sequence
  FSD288
KWKLLKWWSRLLKWWGGSGGGSQARAQARQAR

SEQ ID NO: 200 - protein, 32 aa, Artificial Sequence
  FSD289
KWKLLKFWSRLLKFWGGSGGGSQARAQARQAR

SEQ ID NO: 201 - protein, 32 aa, Artificial Sequence
  FSD290
KWKLLKLYSRLLKLYGGSGGGSQARAQARQAR

SEQ ID NO: 202 - protein, 32 aa, Artificial Sequence
  FSD291
KWKLLKLFSRLLKLFGGSGGGSQARAQARQAR

SEQ ID NO: 203 - protein, 32 aa, Artificial Sequence
  FSD292
KWKLLSLWSSLLSLWGGSGGGSQARAQARQAR

SEQ ID NO: 204 - protein, 32 aa, Artificial Sequence
  FSD293
KWKLLSLWSRLLSLWGGSGGGSQARAQARQAR

SEQ ID NO: 205 - protein, 32 aa, Artificial Sequence
  FSD2294
KWKLLKLWSSLLKLWGGSGGGSQARAQARQAR

SEQ ID NO: 206 - protein, 31 aa, Artificial Sequence
  FSD295
KWKLLKLWSLLKLWGGSGGGSQARAQARQAR

SEQ ID NO: 207 - protein, 34 aa, Artificial Sequence
  FSD296
KWKLLKLWSRLLKLWQQGGSGGGSQARAQARQAR

SEQ ID NO: 208 - protein, 34 aa, Artificial Sequence
  FSD297
KWKLLKLWSRLLKLWNNGGSGGGSQARAQARQAR

SEQ ID NO: 209 - protein, 32 aa, Artificial Sequence
  FSD298
SWSLLKLWSRLLKLWGGSGGGSQARAQARQAR

SEQ ID NO: 210 - protein, 30 aa, Artificial Sequence
  FSD299
KWKLLKLWSRLLKLWIKIFGQARAQARQAR

SEQ ID NO: 211 - protein, 30 aa, Artificial Sequence
  FSD300
KWKLLKLWSRLLKLWWRIFGQARAQARQAR

SEQ ID NO: 212 - protein, 29 aa, Artificial Sequence
  FSD301
GGSGGGSKWKLLKLWSRLLKLWGGSGGGS

SEQ ID NO: 213 - protein, 28 aa, Artificial Sequence
  FSD302
KWKLLKLWSRLLKLWGGGQARAQARQAR

SEQ ID NO: 214 - protein, 26 aa, Artificial Sequence
  FSD303
KWKLLKLWSRLLKLWGQARAQARQAR

SEQ ID NO: 215 - protein, 25 aa, Artificial Sequence
  FSD304
KWKLLKLWSRLLKLWQARAQARQAR

SEQ ID NO: 216 - protein, 32 aa, Artificial Sequence
  FSD305
KWKLLKLWSRLLKLWGGGGGGGQARAQARQAR

SEQ ID NO: 217 - protein, 28 aa, Artificial Sequence
  FSD306
KWKLLKLWSRLLKLWGGSGGGSQARQAR

SEQ ID NO: 218 - protein, 25 aa, Artificial Sequence
  FSD307
KWKLLKLWSRLLKLWGGSGGGSQAR

SEQ ID NO: 219 - protein, 32 aa, Artificial Sequence
  FSD308
KWKLLKLWSRLLKLWGGSGGGSGARAGARGAR

SEQ ID NO: 220 - protein, 32 aa, Artificial Sequence
  FSD309
KWKLLKLWSRLLKLWGGSGGGSQAGAQAGQAG

SEQ ID NO: 221 - protein, 32 aa, Artificial Sequence
  FSD310
KWKLLKLWSRLLKLWGGSGGGSQGRGQGRQGR

SEQ ID NO: 222 - protein, 30 aa, Artificial Sequence
  FSD311
KWKLLKLWSRLLKLWGGSGGGSRGGRGGGR

SEQ ID NO: 223 - protein, 28 aa, Artificial Sequence
  FSD312
WIRLFTKLWIFQQGGSGGGSKRIKAKRA

SEQ ID NO: 224 - protein, 29 aa, Artificial Sequence
  FSD313
WIRLFSRLWRIFQQGGSGGGSKRIKAKRA

SEQ ID NO: 225 - protein, 32 aa, Artificial Sequence
  FSD314
KWKWIRLFSRLWRIFQQGGSGGGSKRIKAKRA

SEQ ID NO: 226 - protein, 31 aa, Artificial Sequence
  FSD315
WIRLFSRLWRIFQQGGSGGGSQARAQARQAR

SEQ ID NO: 227 - protein, 34 aa, Artificial Sequence
  FSD316
KWKWIRLFSRLWRIFQQGGSGGGSQARAQARQAR

SEQ ID NO: 228 - protein, 30 aa, Artificial Sequence
  FSD317
WIRLFTKLWQIFQQGGGSGGGSARAARQAR

SEQ ID NO: 229 - protein, 30 aa, Artificial Sequence
  FSD318
WIRLFTKLWRIFQQGGGSGGGSARAARQAR

SEQ ID NO: 230 - protein, 28 aa, Artificial Sequence
  FSD319
KWKLLKLWSRLLKLWGGSGGGSAAAAQK

SEQ ID NO: 231 - protein, 28 aa, Artificial Sequence
  FSD320
KWKLLKLWSRLLKLWGGSGGGSAAAAQQ

SEQ ID NO: 232 - protein, 24 aa, Artificial Sequence
  FSD321
KWKLAKAWSRAIKIWGARAQARQA

SEQ ID NO: 233 - protein, 30 aa, Artificial Sequence
  FSD322
KWKLAKAWSRALKLWGGSGGGSARAQARQA

SEQ ID NO: 234 - protein, 22 aa, Artificial Sequence
  FSD323
WIRLFTRLIKIWGQRRLKAKRA

SEQ ID NO: 235 - protein, 22 aa, Artificial Sequence
  FSD324
WARAFARAWRIFQQRRLKAKRA

SEQ ID NO: 236 - protein, 25 aa, Artificial Sequence
  FSD325
WARAFARAWRIFQQRRLARAARQAR

SEQ ID NO: 237 - protein, 23 aa, Artificial Sequence
  FSD326
LARAFARAIKIFGQARAQARQAR

SEQ ID NO: 238 - protein, 23 aa, Artificial Sequence
  FSD327
LARAFARAIKIFGRRLKAKRAKA

SEQ ID NO: 239 - protein, 23 aa, Artificial Sequence
  FSD328
LARAFARAIKIFGRRLGARAQAR

SEQ ID NO: 240 - protein, 23 aa, Artificial Sequence
  FSD330
LARAFARALLKLWGQRRLKAKRA

SEQ ID NO: 241 - protein, 21 aa, Artificial Sequence
  FSD331
KWKLARAFARAGQRRLKAKRA

SEQ ID NO: 242 - protein, 22 aa, Artificial Sequence
  FSD332
KWKLARAFARAGRRLGARAQAR

SEQ ID NO: 243 - protein, 32 aa, Artificial Sequence
  FSD333
KWKLLRLLLRLLKKLGGSGGGSQARAQARQAR

SEQ ID NO: 244 - protein, 32 aa, Artificial Sequence
  FSD334
KWKLLRWLWRLLKKLGGSGGGSQARAQARQAR

SEQ ID NO: 245 - protein, 32 aa, Artificial Sequence
  FSD335
KWKLARLLLRALKKLGGSGGGSQARAQARQAR

SEQ ID NO: 246 - protein, 32 aa, Artificial Sequence
  FSD336
KWKLLRLFLRLFKKLGGSGGGSQARAQARQAR

SEQ ID NO: 247 - protein, 32 aa, Artificial Sequence
  FSD337
KWKLARWLWRALKKLGGSGGGSQARAQARQAR

SEQ ID NO: 248 - protein, 32 aa, Artificial Sequence
  FSD338
KWKLLRWFWRLFKKLGGSGGGSQARAQARQAR

SEQ ID NO: 249 - protein, 32 aa, Artificial Sequence
  FSD339
KWKLARLFLRAFKKLGGSGGGSQARAQARQAR

SEQ ID NO: 250 - protein, 32 aa, Artificial Sequence
  FSD340
KWKLARWFWRAFKKLGGSGGGSQARAQARQAR

SEQ ID NO: 251 - protein, 37 aa, Artificial Sequence
  FSD341
LLKIWSRLIKIWTQGRRLGGSGGGSYARALRRQARTG

SEQ ID NO: 252 - protein, 34 aa, Artificial Sequence
  FSD342
KWKLARWFWRAFKKLGGSGGGSYARALRRQARTG

SEQ ID NO: 253 - protein, 30 aa, Artificial Sequence
  FSD343
KWKLLQLWSRLLQLWGGSGGGSARAARQAR

SEQ ID NO: 254 - protein, 30 aa, Artificial Sequence
  FSD344
QWQLLKLWSRLLKLWGGSGGGSARAARQAR

SEQ ID NO: 255 - protein, 30 aa, Artificial Sequence
  FSD345
KLKLLKLWSRLLKLWGGSGGGSARAARQAR

SEQ ID NO: 256 - protein, 30 aa, Artificial Sequence
  FSD346
KFKLLKLFSRLLKLFGGSGGGSARAARQAR

SEQ ID NO: 257 - protein, 30 aa, Artificial Sequence
  FSD347
KWKLLKLLSRLLKLLGGSGGGSARAARQAR

SEQ ID NO: 258 - protein, 30 aa, Artificial Sequence
  FSD348
KWKLLKLLSRLLKLLGGGGGGGARAARQAR

SEQ ID NO: 259 - protein, 30 aa, Artificial Sequence
  FSD349
KWKWLKLWSRLWKLWGGSGGGSARAARQAR

SEQ ID NO: 260 - protein, 30 aa, Artificial Sequence
  FSD350
KWKLLKFWSRLLKFWGGSGGGSARAARQAR

SEQ ID NO: 261 - protein, 30 aa, Artificial Sequence
  FSD351
KWKLLKLFSRLFKLWGGSGGGSARAARQAR

SEQ ID NO: 262 - protein, 30 aa, Artificial Sequence
  FSD352
KWKLLKLWSRLIKIWGGSGGGSARAARQAR

SEQ ID NO: 263 - protein, 30 aa, Artificial Sequence
  FSD353
KWKLLKLQSRLLKLQGGSGGGSARAARQAR

SEQ ID NO: 264 - protein, 25 aa, Artificial Sequence
  FSD354
KWKLLKLWSRLLKLWGGSGGGSQGR

SEQ ID NO: 265 - protein, 25 aa, Artificial Sequence
  FSD355
KWKLLKLWSRLLKLWGGSGGGSGAR

SEQ ID NO: 266 - protein, 25 aa, Artificial Sequence
  FSD356
KWKLLKLWSRLLKLWGGSGGGSQAG

SEQ ID NO: 267 - protein, 25 aa, Artificial Sequence
  FSD357
KWKLLKLWSRLLKLWGGSGGGSRRR

SEQ ID NO: 268 - protein, 30 aa, Artificial Sequence
  FSD358
KWKLLHLWSRLLHLWGGSGGGSARAARQAR

SEQ ID NO: 269 - protein, 30 aa, Artificial Sequence
  FSD359
KWKLLKLWSKLLKLWGGGGGGGAKAAKQAK

SEQ ID NO: 270 - protein, 30 aa, Artificial Sequence
  FSD360
RWRLLRLWSRLLRLWGGGGGGGARAARQAR

SEQ ID NO: 271 - protein, 27 aa, Artificial Sequence
  FSD361
LLKLWSKLLKLWGGGGGGGAKAAKQAK

SEQ ID NO: 272 - protein, 27 aa, Artificial Sequence
  FSD362
LLRLWSRLLRLWGGGGGGGARAARQAR

SEQ ID NO: 273 - protein, 23 aa, Artificial Sequence
  FSD363
LLKLWSKLLKLWGGGAKAAKQAK

SEQ ID NO: 274 - protein, 23 aa, Artificial Sequence
  FSD364
LLRLWSRLLRLWGGGARAARQAR

SEQ ID NO: 275 - protein, 21 aa, Artificial Sequence
  FSD365
KWKLLKLWSRLLKLWGGGQAR

SEQ ID NO: 276 - protein, 18 aa, Artificial Sequence
  FSD366
KWKLWSRLLKLWGGGQAR

SEQ ID NO: 277 - protein, 19 aa, Artificial Sequence
  FSD367
KWKLLKLWSRLLKGGGQAR

SEQ ID NO: 278 - protein, 33 aa, Artificial Sequence
  FSD368
KWKLARAFARASRLLKLWGGSGGGSARAARQAR

SEQ ID NO: 279 - protein, 30 aa, Artificial Sequence
  FSD369
KWKLARAFARALKLWGGSGGGSARAARQAR

SEQ ID NO: 280 - protein, 30 aa, Artificial Sequence
  FSD370
KWKLLKLWSRLLKKLGGSGGGSARAARQAR

SEQ ID NO: 281 - protein, 32 aa, Artificial Sequence
  FSD371
KWKLLKLWSRLLKLWQQGGSGGGSARAARQAR

SEQ ID NO: 282 - protein, 36 aa, Artificial Sequence
  FSD372
KWKLARAFARAIKKLNNGGSGGGSYARALRRQARTG

SEQ ID NO: 283 - protein, 26 aa, Artificial Sequence
  FSD373
GGSGGGSLLKLWSRLLKLWGGSGGGS

SEQ ID NO: 284 - protein, 32 aa, Artificial Sequence
  FSD374
GGSGGGSLLKIWSRLIKIWTQGRRLGGSGGGS

SEQ ID NO: 285 - protein, 29 aa, Artificial Sequence
  FSD375
GGSGGGSKWKLARAFARAIKKLGGSGGGS

SEQ ID NO: 286 - protein, 26 aa, Artificial Sequence
  FSD376
GGSGGGSLARAFARAIKIFGGSGGGS

SEQ ID NO: 287 - protein, 21 aa, Artificial Sequence
  FSD377
GGGKWKLLKLWSRLLKLWGGG

SEQ ID NO: 288 - protein, 29 aa, Artificial Sequence
  FSD378
GGSGGGSKWKWIRLFSRWIRLFGGSGGGS

SEQ ID NO: 289 - protein, 30 aa, Artificial Sequence
  FSD379
KWKLSKLWSKLSKLWGGSGGGSARAARQAR

SEQ ID NO: 290 - protein, 32 aa, Artificial Sequence
  FSD381
LLKLAKALAKALKLLGGSGGGSQARAQARQAR

SEQ ID NO: 291 - protein, 26 aa, Artificial Sequence
  FSD382
LLKLAKALAKALKLLGQARAQARQAR

SEQ ID NO: 292 - protein, 29 aa, Artificial Sequence
  FSD383
LLKLLLKLLKLLGGSGGGSQARAQARQAR

SEQ ID NO: 293 - protein, 23 aa, Artificial Sequence
  FSD384
LAKALAKALKLLGQARAQARQAR

SEQ ID NO: 294 - protein, 29 aa, Artificial Sequence
  FSD385
LLKLLKLLLKLLGGSGGGSQARAQARQAR

SEQ ID NO: 295 - protein, 27 aa, Artificial Sequence
  FSD386
LLKLLKLLLKLLKLLGGGGKGGGKGGK

SEQ ID NO: 296 - protein, 18 aa, Artificial Sequence
  FSD387
QLQLLRLLLRLLKKLQLQ

SEQ ID NO: 297 - protein, 34 aa, Artificial Sequence
  FSD388
KWKLARAFSRAIKLLGGSGGGSYARALRRQARTG

SEQ ID NO: 298 - protein, 34 aa, Artificial Sequence
  FSD389
KWKLAKAFSKAIKLLGGSGGGSYAKALKKQAKTG

SEQ ID NO: 299 - protein, 17 aa, Artificial Sequence
  FSD390
KWKLWSKLLKLWSKLWK

SEQ ID NO: 300 - protein, 31 aa, Artificial Sequence
  FSD391
GGKGGKGGKWKLLKLWSRLLKLWGGKGGKGG

SEQ ID NO: 301 - protein, 31 aa, Artificial Sequence
  FSD392
GGWGGWGGKWKLLKLWSRLLKLWGGWGGWGG

SEQ ID NO: 302 - protein, 30 aa, Artificial Sequence
  FSD393
RAQRAARASGGGSGGWLKLLRSWLKLLKWK

SEQ ID NO: 303 - protein, 40 aa, Artificial Sequence
  FSD394
KWKLARAFARAIKIFGGSGGGSGGGKWKLARAFARAIKIF

SEQ ID NO: 304 - protein, 32 aa, Artificial Sequence
  FSD395
KLKLLKLLLKLLKKLGGSGGGSQAKAQAKQAK

SEQ ID NO: 305 - protein, 32 aa, Artificial Sequence
  FSD396
KLKLAKLLLKALKKLGGSGGGSQAKAQAKQAK

SEQ ID NO: 306 - protein, 32 aa, Artificial Sequence
  FSD397
KLKLAKALAKALKKLGGSGGGSQAKAQAKQAK

SEQ ID NO: 307 - protein, 32 aa, Artificial Sequence
  FSD398
KLKLLKALAKLLKKAGGSGGGSQAKAQAKQAK

SEQ ID NO: 308 - protein, 32 aa, Artificial Sequence
  FSD399
KLKLAKALLKALKKLGGSGGGSQAKAQAKQAK

SEQ ID NO: 309 - protein, 32 aa, Artificial Sequence
  FSD400
KLKAAKALAKALKALGGSGGGSQAKAQAKQAK

SEQ ID NO: 310 - protein, 37 aa, Artificial Sequence
  FSD401
GGSGGGSKWKLLKLWSRLLKLWGGSGGGSARAARQAR

SEQ ID NO: 311 - protein, 29 aa, Artificial Sequence
  FSD402
LLKLLLKLLKKLGGSGGGSQAKAQAKQAK

SEQ ID NO: 312 - protein, 29 aa, Artificial Sequence
  FSD403
LAKALAKALKKLGGSGGGSQAKAQAKQAK

SEQ ID NO: 313 - protein, 29 aa, Artificial Sequence
  FSD404
KLKLLLKLLKKLGGSGGGSQAKAQAKQAK

SEQ ID NO: 314 - protein, 30 aa, Artificial Sequence
  FSD406
KLKLLKLLLKLLKKLGGSGGGSAKAQAKQA

SEQ ID NO: 315 - protein, 30 aa, Artificial Sequence
  FSD407
KLKLLKLLLKLLKKLGGSGGGSAKAAKQAK

SEQ ID NO: 316 - protein, 25 aa, Artificial Sequence
  FSD408
KLKLLKLLLKLLKKLGGSGGGSQAG

SEQ ID NO: 317 - protein, 25 aa, Artificial Sequence
  FSD409
KLKLAKALAKALKKLGGSGGGSQAG

SEQ ID NO: 318 - protein, 32 aa, Artificial Sequence
  FSD410
KLKLLKLLLKLLKKLGGSGGGSLAKALAKLAK

SEQ ID NO: 319 - protein, 32 aa, Artificial Sequence
  FSD411
KLKLLKLLLKLLKKLGGSGGGSQAKALAKQAK

SEQ ID NO: 320 - protein, 25 aa, Artificial Sequence
  FSD412
KLKLLKLLLKLLKKLGGSGGGSLAG

SEQ ID NO: 321 - protein, 32 aa, Artificial Sequence
  FSD413
KLKLAKALAKALKKLGGSGGGSQAKALAKQAK

SEQ ID NO: 322 - protein, 36 aa, Artificial Sequence
  FSD414
LLKKLLHLLHSLLQNLKKLGGSGGGSQAKAQAKQAK

SEQ ID NO: 323 - protein, 36 aa, Artificial Sequence
  FSD415
LIRKWIHLIHSWFQNLRRLGGSGGGSQAKAQAKQAK

SEQ ID NO: 324 - protein, 29 aa, Artificial Sequence
  FSD416
GGSGGGSKWKLAKAWSRALKLWGGSGGGS

SEQ ID NO: 325 - protein, 26 aa, Artificial Sequence
  FSD417
GGSGGGSLAKAWSRALKLWGGSGGGS

SEQ ID NO: 326 - protein, 29 aa, Artificial Sequence
  FSD418
GGSGGGSKLKLLKLLLKLLKKLGGSGGGS

SEQ ID NO: 327 - protein, 29 aa, Artificial Sequence
  FSD419
GGSGGGSKLKLAKALAKALKKLGGSGGGS

SEQ ID NO: 328 - protein, 33 aa, Artificial Sequence
  FSD421
GGSGGGSLLKKLLHLLHSLLQNLKKLGGSGGGS

SEQ ID NO: 329 - protein, 27 aa, Artificial Sequence
  FSD422
HHHHHHKWKLARAFARAIKKLHHHHHH

SEQ ID NO: 330 - protein, 24 aa, Artificial Sequence
  FSD423
HHHHHHLARAFARAIKIFHHHHHH

SEQ ID NO: 331 - protein, 29 aa, Artificial Sequence
  FSD424
KWKLARAFARAIKKLGGSGGGSGGSGGGS

SEQ ID NO: 332 - protein, 32 aa, Artificial Sequence
  FSD425
KLKLAKALAKALKLLGGSGGGSQAKAQAKQAK

SEQ ID NO: 333 - protein, 33 aa, Artificial Sequence
  FSD426
KLKLAKALAKALKKLGGSGGGSKKLKAKKALKA

SEQ ID NO: 334 - protein, 30 aa, Artificial Sequence
  FSD427
LAKALAKALKKLGGSGGGSKKLKAKKALKA

SEQ ID NO: 335 - protein, 30 aa, Artificial Sequence
  FSD428
KLKLAKALAKALKKLGGSGGGSKKLKAKKA

SEQ ID NO: 336 - protein, 28 aa, Artificial Sequence
  FSD429
KLKLAKALAKALKKLGGSGGGSKKLKAK

SEQ ID NO: 337 - protein, 33 aa, Artificial Sequence
  FSD430
KLKLAKALAKALKLLGGSGGGSKKLKAKLALKA

SEQ ID NO: 338 - protein, 34 aa, Artificial Sequence
  FSD431
KWKLAKAFAKAIKKLGGSGGGSYAKALKKQAKTG

SEQ ID NO: 339 - protein, 32 aa, Artificial Sequence
  FSD432
KWKLARAFARAIKKLGGSGGGSQAKAQAKQAK

SEQ ID NO: 340 - protein, 34 aa, Artificial Sequence
  FSD433
KLKLAKALAKALKKLGGSGGGSYARALRRQARTG

SEQ ID NO: 341 - protein, 34 aa, Artificial Sequence
  FSD434
KWKLAKAFAKAIKKLGGSGGGSGGKGGKKQGKTG

SEQ ID NO: 342 - protein, 32 aa, Artificial Sequence
  FSD435
  MISC_FEATURE (1)..(32): Xaa is L-2,4-diaminobutyric acid
XLXLLXLLLXLLXXLGGSGGGSQAXAQAXQAX

SEQ ID NO: 343 - protein, 22 aa, Artificial Sequence
  FSD436
  MISC_FEATURE (1)..(22): Xaa is (2-naphthyl)-L-alanine
LARAXARAIKIXGQRRLKAKRA

SEQ ID NO: 344 - protein, 34 aa, Artificial Sequence; identical to SEQ ID NO: 13
  FSD438
  MISC_FEATURE (1)..(1): N-ter octanoic acid

SEQ ID NO: 345 - protein, 17 aa, Artificial Sequence
  His-PTD4
HHHHHHYARAAARQARA

SEQ ID NO: 346 - protein, 15 aa, Artificial Sequence
  FSD92
LLKIWSRLIKIWTQG

SEQ ID NO: 347 - protein, 14 aa, Artificial Sequence
  C(LLKK)3C
CLLKKLLKKLLKKC

SEQ ID NO: 348 - protein, 18 aa, Artificial Sequence
  CM18
KWKLFKKIGAVLKVLTTG

SEQ ID NO: 349 - protein, 8 aa, Artificial Sequence
  FSD10-8
LARAFARA

SEQ ID NO: 350 - protein, 12 aa, Artificial Sequence
  FSD10-12-1
LARAFARAIKKL

SEQ ID NO: 351 - protein, 12 aa, Artificial Sequence
  FSD10-12-2
KWKLARAFARAI

SEQ ID NO: 352 - protein, 15 aa, Artificial Sequence
  FSD10-15
KWKLARAFARAIKKL

SEQ ID NO: 353 - protein, 8 aa, Artificial Sequence
  FSD418-8
KLLKLLLK

SEQ ID NO: 354 - protein, 12 aa, Artificial Sequence
  FSD418-12-1
KLKLLKLLLKKL

SEQ ID NO: 355 - protein, 12 aa, Artificial Sequence
  FSD418-12-2
LLKLLLKLLKKL

SEQ ID NO: 356 - protein, 15 aa, Artificial Sequence
  FSD418-15
KLKLLKLLLKLLKKL

SEQ ID NO: 357 - protein, 19 aa, Artificial Sequence
  FSD418-19
KLKLLKLLLKLLLKLLKKL

SEQ ID NO: 358 - protein, 20 aa, Artificial Sequence
  FSD439
LAKALAKALKKLGQAKAQAK

SEQ ID NO: 359 - protein, 32 aa, Artificial Sequence
  FSD440
GGSGGGSKWKLFKKIGAVLKVLTTGGGSGGGS

SEQ ID NO: 360 - protein, 40 aa, Artificial Sequence
  FSD441
GGSGGGSKKALLALALHHLAHLALHLALALKKAGGSGGGS

SEQ ID NO: 361 - protein, 37 aa, Artificial Sequence
  FSD442
GGSGGGSGLFGAIAGFIENGWEGMIDGWYGGGSGGGS

SEQ ID NO: 362 - protein, 44 aa, Artificial Sequence
  FSD443
GGSGGGSWEAKLAKALAKALAKHLAKALAKALKACEAGGSGGGS

SEQ ID NO: 363 - protein, 30 aa, Artificial Sequence
  KALA
WEAKLAKALAKALAKHLAKALAKALKACEA

SEQ ID NO: 364 - protein, 18 aa, Artificial Sequence
  FSD444
KHKHKHKHKHKHKHKHKH

SEQ ID NO: 365 - protein, 26 aa, Artificial Sequence
  LAH4
KKALLALALHHLAHLALHLALALKKA

SEQ ID NO: 366 - protein, 16 aa, Artificial Sequence
  Penetratin
RQIKIWFQNRRMKWKK

SEQ ID NO: 367 - protein, 11 aa, Artificial Sequence
  PTD4
YARAAARQARA

SEQ ID NO: 368 - protein, 11 aa, Artificial Sequence
  TAT
YGRKKRRQRRR

SEQ ID NO: 369 - protein, 30 aa, Artificial Sequence
  FSD445
GWGLLKLWSRLLKLWGGSGGGSARAARQAR

SEQ ID NO: 370 - protein, 29 aa, Artificial Sequence
  FSD446
KWKLLKLWSRLLKLWGGSGGGSHTSDQTN

SEQ ID NO: 371 - protein, 18 aa, Artificial Sequence
  FSD447
RHRHRHRHRHRHRHRHRH

SEQ ID NO: 372 - RNA, 20 nt, Artificial Sequence
  crRNA Beta-2 microglobulin (B2M) for Cas9
gaguagcgcgagcacagcua

SEQ ID NO: 373 - RNA, 23 nt, Artificial Sequence
  crRNA Beta-2 microglobulin (B2M) for Cas12a (Cpf1)
agugggggugaauucaguguagu

SEQ ID NO: 374 - protein, 36 aa, Artificial Sequence
  CM18-L2-PTD4
KWKLFKKIGAVLKVLTTGGGSGGGSYARAAARQARA

SEQ ID NO: 375 - protein, 50 aa, Artificial Sequence
  His-CM18-Transportan
HHHHHHKWKLFKKIGAVLKVLTTGGWTLNSAGYLLKINLKALAALAKKIL

SEQ ID NO: 376 - protein, 29 aa, Artificial Sequence
  CM18-TAT
KWKLFKKIGAVLKVLTTGYGRKKRRQRRR

SEQ ID NO: 377 - protein, 33 aa, Artificial Sequence
  His-CM18-9Arg
HHHHHHKWKLFKKIGAVLKVLTTGRRRRRRRRR

SEQ ID NO: 378 - protein, 35 aa, Artificial Sequence
  His-CM18-TAT
HHHHHHKWKLFKKIGAVLKVLTTGYGRKKRRQRRR

SEQ ID NO: 379 - protein, 22 aa, Artificial Sequence
  FSD448
KWKLARAFARAIKKLGGSGGGS

SEQ ID NO: 380 - protein, 26 aa, Artificial Sequence
  Linker-(FSD10-Cter)-Linker
GGSGGGSYARALRRQARTGGGSGGGS

SEQ ID NO: 381 - protein, 12 aa, Artificial Sequence
  FSD10-Cter
YARALRRQARTG